(19) United States
(12) Patent Application Publication
(10) Pub. No.: US 2025/0002486 A1
(43) Pub. Date: Jan. 2, 2025

(54) CYCLIC SULFONAMIDE RIBONUCLEOTIDE REDUCTASE (RNR) INHIBITORS AND USES THEREOF

(71) Applicant: Boundless Bio, Inc., San Diego, CA (US)

(72) Inventors: Anthony B. PINKERTON, Rancho Santa Fe, CA (US); Jacques MAUGER, San Diego, CA (US); Yen Pham Hong TRUONG, San Diego, CA (US); Rachelle Janette ELSDON, San Diego, CA (US); Stephen Todd MEYER, San Diego, CA (US)

(21) Appl. No.: 18/692,503

(22) PCT Filed: Sep. 15, 2022
(86) PCT No.: PCT/US2022/043660

§ 371 Date: Mar. 15, 2024

Related U.S. Application Data
(60) Provisional application No. 63/245,718, filed on Sep. 17, 2021.

(51) Int. Cl. C07D 417/06; A61K 31/428; A61K 31/5415; A61K 31/542; A61K 31/554; A61K 45/06; C07D 513/04

(52) U.S. Cl. CPC C07D 417/06; A61K 31/428; A61K 31/5415; A61K 31/542; A61K 31/554; A61K 45/06; C07D 513/04

(57) ABSTRACT

Provided herein are compounds and methods for the treatment of cancer. The methods include administering to a subject in need a therapeutically effective amount of a cyclic sulfonamide RNR inhibitor disclosed herein.

### CROSS-REFERENCE

This application claims the benefit of U.S. Provisional Application Ser. No. 63/245,718 filed Sep. 17, 2021 which is hereby incorporated by reference in its entirety.

### FIELD OF THE INVENTION

Described herein are compounds, methods of making such compounds, pharmaceutical compositions, and medicaments comprising such compounds, and methods of using such compounds for inhibiting ribonucleotide reductase (RNR).

### BACKGROUND OF THE INVENTION

Ribonucleotide reductase (RNR), also known as ribonucleotide diphosphate reductase (rNDP), is composed of a hetero-oligomer of a large subunit M1 and a small subunit M2, and expression of both is required for enzyme activity. RNR is a highly regulated enzyme in the deoxyribonucleotide synthesis pathway that is ubiquitously present in human, bacteria, yeast, and other organisms. RNR is responsible for the de novo conversion of ribonucleotide diphosphate to 2′-deoxyribonucleotide diphosphate, a process that is essential for DNA synthesis and repair. RNR is directly involved in DNA synthesis and repair, tumor growth, metastasis, and drug resistance. In various types of solid tumors and blood cancers, numerous correlations have been reported with overexpression of M2 and their prognosis. In addition, cell growth inhibition by inhibiting RNR and anti-tumor effect in vivo have been reported in cell lines derived from several cancer types and in nonclinical models.

The proliferation of cancer cells requires excess deoxyribonucleotide triphosphates (dNTPs) for DNA synthesis. Therefore, an increase in RNR activity is necessary as it helps provide extra dNTPs for DNA replication in primary and metastatic cancer cells. Because of this critical role in DNA synthesis, RNR represents an important target for cancer therapy. However, existing chemotherapies that target RNR are nucleoside-based analogs. Hence, they are promiscuous, leading to nonspecific binding of other nucleoside binding proteins which results in unwanted side effects. Therefore, there is a need for compositions and methods for specifically targeting and inhibiting RNR activity in neoplastic cells in the treatment of cancer.

### BRIEF SUMMARY OF THE INVENTION

Described herein are RNR inhibitors that are useful in treating cancer.

Disclosed herein is a compound of Formula (I), or a pharmaceutically acceptable salt, solvate, tautomer, or stereoisomer thereof:

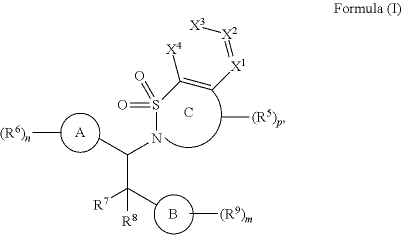

wherein:
X¹ is N or CR¹;
X² is N or CR²;
X³ is N or CR³;
X⁴ is N or CR⁴;
R¹, R², R³, and R⁴ are independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
Ring C is a 4- to 8-membered heterocycloalkyl optionally comprising 1 or 2 additional heteroatoms selected from the group consisting of O, S, and N;
each R⁵ is independently deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl;
or 2 R⁵ on the same carbon are taken together to form an oxo;
p is 0-4;
Ring A is a 5-membered ring comprising 1-4 heteroatoms selected from the group consisting of O, S, and N;
each R⁶ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁶ on the same atom are taken together to form an oxo;
n is 0-3;
R⁷ is hydrogen, deuterium, halogen, —CN, —NO₂, —OH, —OR^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl;
R⁸ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
Ring B is cycloalkyl, heterocycloalkyl, aryl, or heteroaryl;
each R⁹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NRC(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
or two R⁹ on the same atom are taken together to form an oxo;
each R^{9a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more of deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{9a} on the same atom are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{b} are taken together with the atom to which they are attached to form a heterocycloalkyl optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, C₁-C₆heteroalkyl.

Also disclosed herein is a compound of Formula (Ia), or a pharmaceutically acceptable salt, solvate, tautomer, or stereoisomer thereof:

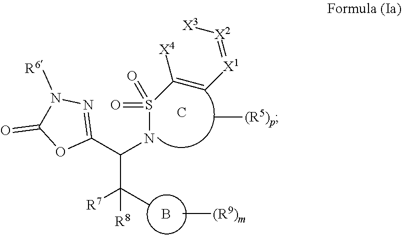

wherein

R^{6′} is hydrogen or C₁-C₆alkyl.

Also disclosed herein is a compound of Formula (Ib), or a pharmaceutically acceptable salt, solvate, tautomer, or stereoisomer thereof:

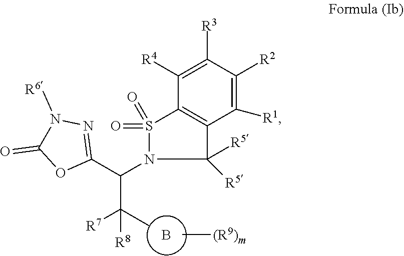

wherein
R^{6′} is hydrogen or C₁-C₆alkyl; and
each R^{5′} is independently hydrogen or R⁵.

Also disclosed herein is a compound of Formula (Ic), or a pharmaceutically acceptable salt, solvate, tautomer, or stereoisomer thereof:

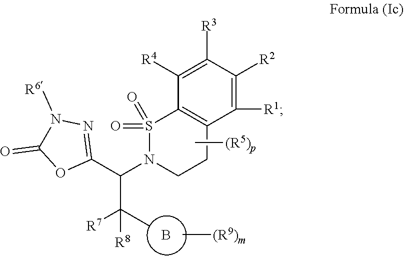

wherein
R^{6′} is hydrogen or C₁-C₆alkyl.

Also disclosed herein is a compound of Formula (Id), or a pharmaceutically acceptable salt, solvate, tautomer, or stereoisomer thereof:

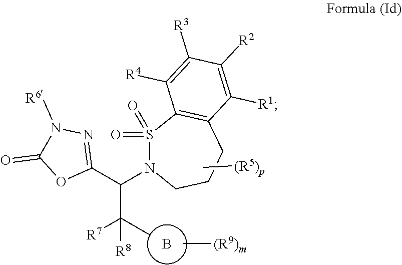

wherein
R^{6′} is hydrogen or C₁-C₆alkyl.

Also disclosed herein is a pharmaceutical composition comprising a compound disclosed herein, or a pharmaceutically acceptable salt, solvate, tautomer, or stereoisomer thereof, and a pharmaceutically acceptable excipient.

Also disclosed herein is a method of treating cancer in a subject, comprising administering to the subject a compound disclosed herein, or a pharmaceutically acceptable salt, solvate, tautomer, or stereoisomer thereof, or a pharmaceutical composition disclosed herein.

Also disclosed herein is a method of inhibiting ribonucleotide reductase in a subject, comprising administering to the subject a compound disclosed herein, or a pharmaceutically acceptable salt, solvate, tautomer, or stereoisomer thereof, or a pharmaceutical composition disclosed herein.

In some embodiments, the inhibition of ribonucleotide reductase occurs in a tumor cell in the subject in need thereof.

Also disclosed herein is a method for treating a tumor or tumor cells in a subject, the method comprising administering a compound disclosed herein, or a pharmaceutically acceptable salt, solvate, tautomer, or stereoisomer thereof, in an amount sufficient to induce replication stress in the tumor or tumor cells; and administering a cancer-targeted therapeutic agent; wherein the tumor or tumor cells have an ecDNA signature; and wherein growth or size of the tumor or growth or number of tumor cells is reduced.

Also disclosed herein is a method of treating an ecDNA-associated tumor or tumor cells comprising administering a compound disclosed herein, or a pharmaceutically acceptable salt, solvate, tautomer, or stereoisomer thereof, to a subject identified as having a tumor or tumor cells having ecDNA, wherein growth or size of the tumor or growth or number of the tumor cells is decreased as a result of treatment.

In some embodiments, the method further comprises administering a cancer-targeted therapeutic agent.

In some embodiments, the cancer-targeted therapeutic agent inhibits a gene or gene product comprised on ecDNA in the tumor or tumor cells.

Also disclosed herein is a method for treating a tumor or tumor cells in a subject, the method comprising administering a compound disclosed herein, or a pharmaceutically acceptable salt, solvate, tautomer, or stereoisomer thereof, in an amount sufficient to induce replication stress in the tumor or tumor cells, wherein the tumor or tumor cells comprises ecDNA or have an ecDNA signature; and wherein growth or size of the tumor or growth or number of tumor cells is reduced.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Oxo” refers to =O.

“Alkyl” refers to an optionally substituted straight-chain, or optionally substituted branched-chain saturated hydrocarbon monoradical having from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₅ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen. In some embodiments, the alkyl is optionally substituted with —COOH, —COOMe, —CONH₂, —CONHMe, or —CONMe₂.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₅ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen. In some embodiments, the alkenyl is optionally substituted with —COOH, —COOMe, —CONH₂, —CONHMe, or —CONMe₂.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₈ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. In some embodiments, the alkynyl is optionally substituted with —COOH, —COOMe, —CONH₂, —CONHMe, or —CONMe₂.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is optionally substituted with —COOH, —COOMe, —CONH₂, —CONHMe, or —CONMe₂.

“Alkoxy” refers to a radical of the formula -Oalkyl where alkyl is as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. n some embodiments, an alkoxy is optionally substituted with oxo, halogen, —CN, —CF₃, —OH, or —OMe. In some embodiments, the alkoxy is optionally substituted with halogen. In some embodiments, the alkoxy is optionally substituted with —COOH, —COOMe, —CONH₂, —CONHMe, or —CONMe₂.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen. In some embodiments, the aryl is optionally substituted with —COOH, —COOMe, —CONH₂, —CONHMe, or —CONMe₂.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₈ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen. In some embodiments, the cycloalkyl is optionally substituted with —COOH, —COOMe, —CONH₂, —CONHMe, or —CONMe₂.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens. Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments, halogen is fluoro or chloro. In some embodiments, halogen is fluoro. In some embodiments, halogen is chloro. In some embodiments, halogen is bromo. In some embodiments, halogen is iodo.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen. In some embodiments, the heteroalkyl is optionally substituted with —COOH, —COOMe, —CONH₂, —CONHMe, or —CONMe₂.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a 3- to 24-membered partially or fully saturated, not fully aromatic ring radical comprising 2 to 23 carbon atoms and from one to 8 heteroatoms selected from the group consisting of nitrogen, oxygen, phosphorous and sulfur. In some embodiments, the heterocycloalkyl comprises 1 or 2 heteroatoms selected from nitrogen and oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen. In some embodiments, the heterocycloalkyl is optionally substituted with —COOH, —COOMe, —CONH₂, —CONHMe, or —CONMe₂.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring comprising at least one heteroatom. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen. In some embodiments, the heteroaryl is optionally substituted with —COOH, —COOMe, —CONH₂, —CONHMe, or —CONMe₂.

The term “one or more” when referring to an optional substituent means that the subject group is optionally substituted with one, two, three, four, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The terms “treat,” “treated,” “treatment,” or “treating” as used herein refers to therapeutic treatment, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “treat,” “treated,” “treatment,” or “treating” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the disclosed methods can provide any amount of any level of treatment of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “ecDNA signature” as used herein, generally refers to one or more characteristics common to tumors or tumor cells that are ecDNA+(contain extrachromosomal DNA (ecDNA)). In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, ecDNA signature includes a detection or identification of ecDNA using an imaging technology. In some cases, ecDNA signature does not include any imaging or direct detection of ecDNA.

### Compounds

Described herein are cyclic sulfonamide RNR inhibitors that are useful for the treatment of cancer.

Disclosed herein is a compound of Formula (I), or a pharmaceutically acceptable salt, solvate, tautomer, or stereoisomer thereof:

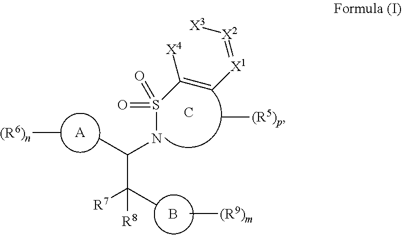

wherein:
X¹ is N or CR¹;
X² is N or CR²;
X³ is N or CR³;
X⁴ is N or CR⁴;
R¹, R², R³, and R⁴ are independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
Ring C is a 4- to 8-membered heterocycloalkyl optionally comprising 1 or 2 additional heteroatoms selected from the group consisting of O, S, and N;
each R⁵ is independently deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl;
or 2 R⁵ on the same carbon are taken together to form an oxo;
p is 0-4;
Ring A is a 5-membered ring comprising 1-4 heteroatoms selected from the group consisting of O, S, and N;
each R⁶ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁶ on the same atom are taken together to form an oxo;
n is 0-3;
R⁷ is hydrogen, deuterium, halogen, —CN, —NO₂, —OH, —OR^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl;
R⁸ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
Ring B is cycloalkyl, heterocycloalkyl, aryl, or heteroaryl;
each R⁹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NRC(═O)R^{a}, —NRC(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
or two R⁹ on the same atom are taken together to form an oxo;
each R^{9a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more of deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{9a} on the same atom are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

In some embodiments of a compound of Formula (I), the compound is of Formula:

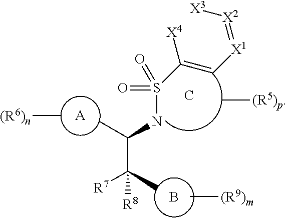

Also disclosed herein is a compound of Formula (I-1), or a pharmaceutically acceptable salt, solvate, tautomer, or stereoisomer thereof:

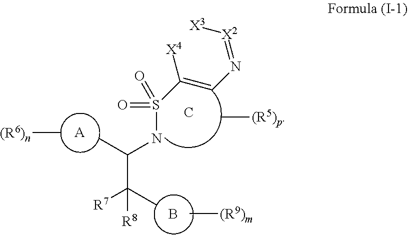

In some embodiments of a compound of Formula (I-1), the compound is of Formula:

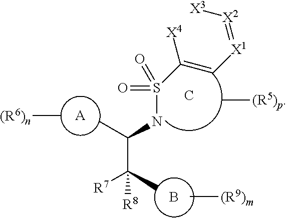

Also disclosed herein is a compound of Formula (I-2), or a pharmaceutically acceptable salt, solvate, tautomer, or stereoisomer thereof:

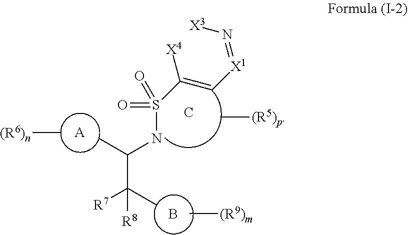

In some embodiments of a compound of Formula (I-2), the compound is of Formula:

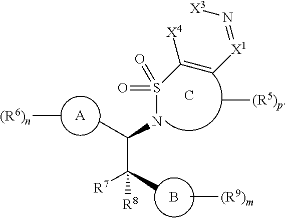

Also disclosed herein is a compound of Formula (I-3), or a pharmaceutically acceptable salt, solvate, tautomer, or stereoisomer thereof:

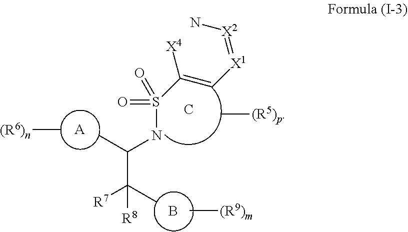

In some embodiments of a compound of Formula (I-3), the compound is of Formula:

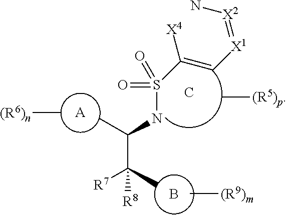

Also disclosed herein is a compound of Formula (I-4), or a pharmaceutically acceptable salt, solvate, tautomer, or stereoisomer thereof:

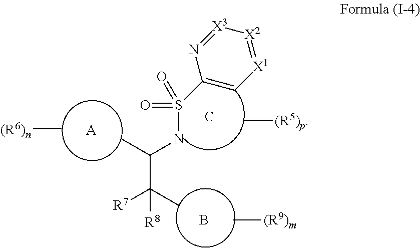

In some embodiments of a compound of Formula (I-4), the compound is of Formula:

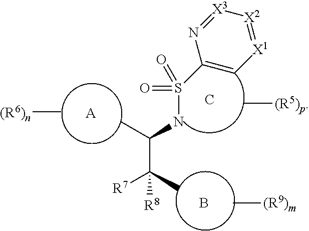

In some embodiments of a compound of Formula (I) or (I-1)-(I-4), Ring A is a 5-membered ring comprising 1 or 2 heteroatoms selected from the group consisting of O, S, and N. In some embodiments of a compound of Formula (I) or (I-1)-(I-4), Ring A is a 5-membered ring comprising 2 or 3 heteroatoms selected from the group consisting of O, S, and N. In some embodiments of a compound of Formula (I) or (I-1)-(I-4), Ring A is a 5-membered ring comprising 2-4 heteroatoms selected from the group consisting of O, S, and N. In some embodiments of a compound of Formula (I) or (I-1)-(I-4), Ring A is a 5-membered ring comprising 1-3 heteroatoms selected from the group consisting of O, S, and N. In some embodiments of a compound of Formula (I) or (I-1)-(I-4), Ring A is a 5-membered ring comprising 3 or 4 heteroatoms selected from the group consisting of O, S, and N.

In some embodiments of a compound of Formula (I) or (I-1)-(I-4), Ring A is a 5-membered ring comprising 1 heteroatom selected from the group consisting of O, S, and N. In some embodiments of a compound of Formula (I) or (I-1)-(I-4), Ring A is a 5-membered ring comprising 2 heteroatoms selected from the group consisting of O, S, and N. In some embodiments of a compound of Formula (I) or (I-1)-(I-4), Ring A is a 5-membered ring comprising 3 heteroatoms selected from the group consisting of O, S, and N. In some embodiments of a compound of Formula (I) or (I-1)-(I-4), Ring A is a 5-membered ring comprising 4 heteroatoms selected from the group consisting of O, S, and N.

In some embodiments of a compound of Formula (I) or (I-1)-(I-4), Ring A is a 5-membered ring comprising 1 or 2 heteroatoms selected from the group consisting of O and N. In some embodiments of a compound of Formula (I) or (I-1)-(I-4), Ring A is a 5-membered ring comprising 2 or 3 heteroatoms selected from the group consisting of O and N. In some embodiments of a compound of Formula (I) or (I-1)-(I-4), Ring A is a 5-membered ring comprising 2-4 heteroatoms selected from the group consisting of O and N. In some embodiments of a compound of Formula (I) or (I-1)-(I-4), Ring A is a 5-membered ring comprising 1-3 heteroatoms selected from the group consisting of O and N. In some embodiments of a compound of Formula (I) or (I-1)-(I-4), Ring A is a 5-membered ring comprising 3 or 4 heteroatoms selected from the group consisting of O and N.

In some embodiments of a compound of Formula (I) or (I-1)-(I-4), Ring A is a 5-membered ring comprising 1 heteroatom selected from the group consisting of O and N. In some embodiments of a compound of Formula (I) or (I-1)-(I-4), Ring A is a 5-membered ring comprising 2 heteroatoms selected from the group consisting of O and N.

In some embodiments of a compound of Formula (I) or (I-1)-(I-4), Ring A is a 5-membered ring comprising 3 heteroatoms selected from the group consisting of O and N. In some embodiments of a compound of Formula (I) or (I-1)-(I-4), Ring A is a 5-membered ring comprising 4 heteroatoms selected from the group consisting of O and N.

In some embodiments of a compound of Formula (I) or (I-1)-(I-4), Ring A is a 5-membered heterocycloalkyl or a 5-membered heteroaryl. In some embodiments of a compound of Formula (I) or (I-1)-(1-4), Ring A is a 5-membered heterocycloalkyl. In some embodiments of a compound of Formula (I) or (I-1)-(I-4), Ring A is a 5-membered heterocycloalkyl comprising one to four heteroatoms selected from the group consisting of O, S, and N. In some embodiments of a compound of Formula (I) or (I-1)-(I-4), Ring A is a 5-membered heterocycloalkyl comprising two to four heteroatoms selected from the group consisting of O, S, and N. In some embodiments of a compound of Formula (I) or (I-1)-(I-4), Ring A is a 5-membered heterocycloalkyl comprising three to four heteroatoms selected from the group consisting of O, S, and N. In some embodiments of a compound of Formula (I) or (I-1)-(I-4), Ring A is a 5-membered heteroaryl. In some embodiments of a compound of Formula (I) or (I-1)-(I-4), Ring A is a 5-membered heteroaryl comprising one to four heteroatoms selected from the group consisting of O, S, and N. In some embodiments of a compound of Formula (I) or (I-1)-(I-4), Ring A is a 5-membered heteroaryl comprising two to four heteroatoms selected from the group consisting of O, S, and N. In some embodiments of a compound of Formula (I) or (I-1)-(I-4), Ring A is a 5-membered heteroaryl comprising three to four heteroatoms selected from the group consisting of O, S, and N.

In some embodiments of a compound of Formula (I) or (I-1)-(I-4), Ring A is a triazole or tetrazole. In some embodiments of a compound of Formula (I) or (I-1)-(I-4), Ring A is a triazole. In some embodiments of a compound of Formula (I) or (I-1)-(I-4), Ring A is a tetrazole. In some embodiments of a compound of Formula (I) or (I-1)-(I-4), Ring A is a 2,3-dihydro-1,3,4-oxadiazole.

In some embodiments of a compound of Formula (I) or (I-1)-(I-4), each R⁶ is independently deuterium, halogen, —CN, —OH, —OR^{a}, —NR^{c}R^{d}, C₁-C₆alkyl, C₁-C₆haloalkyl, or C₁-C₆deuteroalkyl; or two R⁶ on the same atom are taken together to form an oxo. In some embodiments of a compound of Formula (I) or (I-1)-(I-4), each R⁶ is independently deuterium, halogen, or C₁-C₆alkyl; or two R⁶ on the same atom are taken together to form an oxo. In some embodiments of a compound of Formula (I) or (I-1)-(I-4), each R⁶ is independently C₁-C₆alkyl; or two R⁶ on the same atom are taken together to form an oxo. In some embodiments of a compound of Formula (I) or (I-1)-(I-4), each R⁶ is independently C₁-C₆alkyl.

In some embodiments of a compound of Formula (I) or (I-1)-(I-4), two R⁶ on the same atom are taken together to form an oxo.

In some embodiments of a compound of Formula (I) or (I-1)-(I-4), n is 0-2. In some embodiments of a compound of Formula (I) or (I-1)-(I-4), n is 0 or 1. In some embodiments of a compound of Formula (I) or (I-1)-(I-4), n is 1 or 2. In some embodiments of a compound of Formula (I) or (I-1)-(I-4), n is 2 or 3. In some embodiments of a compound of Formula (I) or (I-1)-(I-4), n is 0. In some embodiments of a compound of Formula (I) or (I-1)-(I-4), n is 1. In some embodiments of a compound of Formula (I) or (I-1)-(I-4), n is 2. In some embodiments of a compound of Formula (I) or (I-1)-(I-4), n is 3.

In some embodiments of a compound of Formula (I), the compound is of Formula (Ia):

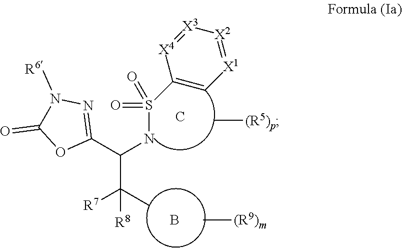

wherein
R^{6′} is hydrogen or C₁-C₆alkyl.

In some embodiments of a compound of Formula (Ia), the compound is of Formula:

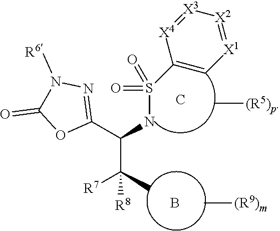

In some embodiments of a compound of Formula (I), (Ia), or (I-1)-(I-4), X¹ is N. In some embodiments of a compound of Formula (I), (Ia), or (I-1)-(I-4), X¹ is CR¹.

In some embodiments of a compound of Formula (I), (Ia), or (I-1)-(I-4), X² is N. In some embodiments of a compound of Formula (I), (Ia), or (I-1)-(I-4), X² is CR².

In some embodiments of a compound of Formula (I), (Ia), or (I-1)-(I-4), X³ is N. In some embodiments of a compound of Formula (I), (Ia), or (I-1)-(I-4), X³ is CR³.

In some embodiments of a compound of Formula (I), (Ia), or (I-1)-(I-4), X⁴ is N. In some embodiments of a compound of Formula (I), (Ia), or (I-1)-(I-4), X⁴ is CR⁴.

In some embodiments of a compound of Formula (I), (Ia), or (I-1)-(I-4), Ring C is a 5- to 7-membered heterocycloalkyl optionally comprising 1 or 2 additional heteroatoms selected from the group consisting of O, S, and N. In some embodiments of a compound of Formula (I), (Ia), or (I-1)-(I-4), Ring C is a 6- to 7-membered heterocycloalkyl optionally comprising 1 or 2 additional heteroatoms selected from the group consisting of O, S, and N. In some embodiments of a compound of Formula (I), (Ia), or (I-1)-(I-4), Ring C is a 5- to 6-membered heterocycloalkyl optionally comprising 1 or 2 additional heteroatoms selected from the group consisting of O, S, and N. In some embodiments of a compound of Formula (I), (Ia), or (I-1)-(1-4), Ring C is a 5-membered heterocycloalkyl optionally comprising 1 or 2 additional heteroatoms selected from the group consisting of O, S, and N. In some embodiments of a compound of Formula (I), (Ia), or (I-1)-(1-4), Ring C is a 6-membered heterocycloalkyl optionally comprising 1 or 2 additional heteroatoms selected from the group consisting of O, S, and N.

In some embodiments of a compound of Formula (I), (Ia), or (I-1)-(I-4), Ring C is a 5- to 7-membered heterocycloalkyl comprising 1 additional heteroatom selected from the group consisting of O, S, and N. In some embodiments of a compound of Formula (I), (Ia), or (I-1)-(I-4), Ring C is a 6- to 7-membered heterocycloalkyl comprising 1 additional heteroatom selected from the group consisting of O, S, and N. In some embodiments of a compound of Formula (I), (Ia), or (I-1)-(I-4), Ring C is a 5- to 6-membered heterocycloalkyl comprising 1 additional heteroatom selected from the group consisting of O, S, and N. In some embodiments of a compound of Formula (I), (Ia), or (I-1)-(I-4), Ring C is a 5-membered heterocycloalkyl comprising 1 additional heteroatom selected from the group consisting of O, S, and N. In some embodiments of a compound of Formula (I), (Ia), or (I-1)-(I-4), Ring C is a 6-membered heterocycloalkyl comprising 1 additional heteroatom selected from the group consisting of O, S, and N. In some embodiments of a compound of Formula (I), (Ia), or (I-1)-(I-4), Ring C is a 7-membered heterocycloalkyl comprising 1 additional heteroatom selected from the group consisting of O, S, and N. In some embodiments of a compound of Formula (I), (Ia), or (I-1)-(I-4), Ring C is a 8-membered heterocycloalkyl comprising 1 additional heteroatom selected from the group consisting of O, S, and N.

In some embodiments of a compound of Formula (I), (Ia), or (I-1)-(I-4), Ring C is a 5- to 7-membered heterocycloalkyl. In some embodiments of a compound of Formula (I), (Ia), or (I-1)-(I-4), Ring C is a 6- to 7-membered heterocycloalkyl. In some embodiments of a compound of Formula (I), (Ia), or (I-1)-(I-4), Ring C is a 5- to 6-membered heterocycloalkyl. In some embodiments of a compound of Formula (I), (Ia), or (I-1)-(I-4), Ring C is a 5-membered heterocycloalkyl. In some embodiments of a compound of Formula (I), (Ia), or (I-1)-(I-4), Ring C is a 6-membered heterocycloalkyl. In some embodiments of a compound of Formula (I), (Ia), or (I-1)-(I-4), Ring C is a 7-membered heterocycloalkyl.

In some embodiments of a compound of Formula (I) or (Ia), the compound is of Formula (Ib):

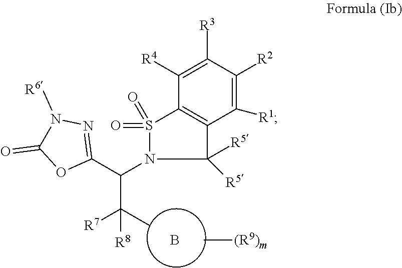

wherein
R^{6′} is hydrogen or C₁-C₆alkyl; and
each R^{5′} is independently hydrogen or R⁵.

In some embodiments of a compound of Formula (Ib), the compound is of Formula:

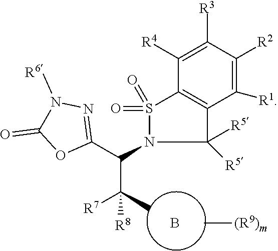

In some embodiments of a compound of Formula (Ib), each R^{5′} is independently hydrogen, deuterium, halogen, —OH, —OR^{a}, —NR^{c}R^{d}, C₁-C₆alkyl, or C₁-C₆haloalkyl; or 2 R⁵ on the same carbon are taken together to form an oxo. In some embodiments of a compound of Formula (Ib), each R^{5′} is independently hydrogen, deuterium, halogen, or C₁-C₆alkyl. In some embodiments of a compound of Formula (Ib), each R^{5′} is independently hydrogen or C₁-C₆alkyl. In some embodiments of a compound of Formula (Ib), each R^{5′} is hydrogen. In some embodiments of a compound of Formula (Ib), each R^{5′} is independently hydrogen or deuterium.

In some embodiments of a compound of Formula (I) or (Ia), the compound is of Formula (Ic):

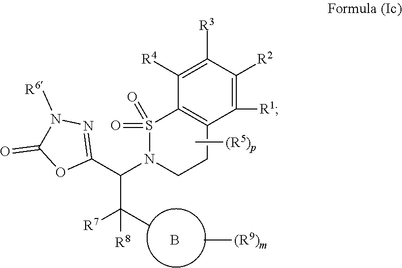

wherein
R^{6′} is hydrogen or C₁-C₆alkyl.

In some embodiments of a compound of Formula (Ic), the compound is of Formula:

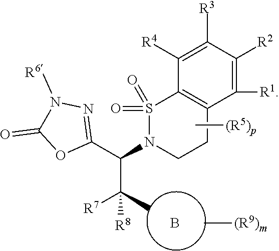

In some embodiments of a compound of Formula (I) or (Ia), the compound is of Formula (Id):

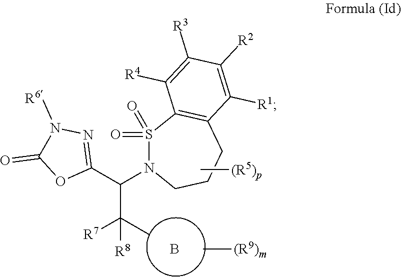

wherein
R^{6′} is hydrogen or C₁-C₆alkyl.

In some embodiments of a compound of Formula (Id), the compound is of Formula:

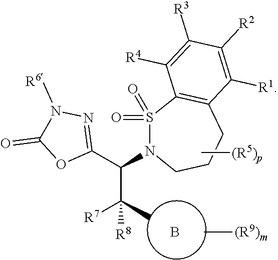

In some embodiments of a compound of Formula (Ia)-(Id), R^{6′} is hydrogen.

In some embodiments of a compound of Formula (I), (Ia)-(Id), or (I-1)-(I-4), each R is independently deuterium, halogen, —OH, —OR^{a}, —NR^{c}R^{d}, C₁-C₆alkyl, or C₁-C₆haloalkyl; or 2 R⁵ on the same carbon are taken together to form an oxo. In some embodiments of a compound of Formula (I), (Ia)-(Id), or (I-1)-(I-4), each R is independently deuterium, halogen, or C₁-C₆alkyl. In some embodiments of a compound of Formula (I), (Ia)-(Id), or (I-1)-(I-4), each R⁵ is independently deuterium. In some embodiments of a compound of Formula (I), (Ia)-(Id), or (I-1)-(I-4), each R⁵ is independently C₁-C₆alkyl.

In some embodiments of a compound of Formula (I), (Ia)-(Id), or (I-1)-(I-4), p is 0 or 1. In some embodiments of a compound of Formula (I), (Ia)-(Id), or (I-1)-(I-4), p is 1 or 2. In some embodiments of a compound of Formula (I), (Ia)-(Id), or (I-1)-(I-4), p is 0. In some embodiments of a compound of Formula (I), (Ia)-(Id), or (I-1)-(I-4), p is 1. In some embodiments of a compound of Formula (I), (Ia)-(Id), or (I-1)-(I-4), p is 2. In some embodiments of a compound of Formula (I), (Ia)-(Id), or (I-1)-(I-4), p is 3.

In some embodiments of a compound of Formula (I), (Ia)-(Id), or (I-1)-(I-4), R¹, R², R³, and R⁴ are independently hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl. In some embodiments of a compound of Formula (I), (Ia)-(Id), or (I-1)-(I-4), R¹, R², R³, and R⁴ are independently hydrogen, deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I), (Ia)-(Id), or (I-1)-(I-4), R¹, R², R³, and R⁴ are independently hydrogen, deuterium, halogen, —CN, —OH, —OR^{a}, —NR^{c}R^{d}, C₁-C₆alkyl, C₁-C₆haloalkyl, or C₁-C₆deuteroalkyl. In some embodiments of a compound of Formula (I), (Ia)-(Id), or (I-1)-(I-4), R¹, R², R³, and R⁴ are independently hydrogen, or halogen, —OR^{a}.

In some embodiments of a compound of Formula (I), (Ia)-(Id), or (I-1)-(I-4), R¹ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I), (Ia)-(Id), or (I-1)-(I-4), R¹ is hydrogen, deuterium, halogen, —CN, —OH, —OR^{a}, —NR^{c}R^{d}, C₁-C₆alkyl, or C₁-C₆haloalkyl. In some embodiments of a compound of Formula (I), (Ia)-(Id), or (I-1)-(I-4), R¹ is hydrogen, halogen, —OH, —OW, C₁-C₆alkyl, or C₁-C₆haloalkyl. In some embodiments of a compound of Formula (I), (Ia)-(Id), or (I-1)-(I-4), R¹ is hydrogen, halogen, or C₁-C₆alkyl. In some embodiments of a compound of Formula (I), (Ia)-(Id), or (I-1)-(I-4), R¹ is hydrogen or halogen. In some embodiments of a compound of Formula (I), (Ia)-(Id), or (I-1)-(I-4), R¹ is hydrogen, halogen, or —OR^{a}. In some embodiments of a compound of Formula (I), (Ia)-(Id), or (I-1)-(I-4), R¹ is halogen.

In some embodiments of a compound of Formula (I), (Ia)-(Id), or (I-1)-(I-4), R² is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I), (Ia)-(Id), or (I-1)-(I-4), R² is hydrogen, deuterium, halogen, —CN, —OH, —OR^{a}, —NR^{c}R^{d}, C₁-C₆alkyl, or C₁-C₆haloalkyl. In some embodiments of a compound of Formula (I), (Ia)-(Id), or (I-1)-(I-4), R² is hydrogen, halogen, —OH, —OR^{a}, C₁-C₆alkyl, or C₁-C₆haloalkyl. In some embodiments of a compound of Formula (I), (Ia)-(Id), or (I-1)-(I-4), R² is hydrogen, halogen, or C₁-C₆alkyl. In some embodiments of a compound of Formula (I), (Ia)-(Id), or (I-1)-(I-4), R² is hydrogen or halogen. In some embodiments of a compound of Formula (I), (Ia)-(Id), or (I-1)-(I-4), R² is hydrogen, halogen, or —OR. In some embodiments of a compound of Formula (I), (Ia)-(Id), or (I-1)-(I-4), R² is halogen.

In some embodiments of a compound of Formula (I), (Ia)-(Id), or (I-1)-(I-4), R³ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I), (Ia)-(Id), or (I-1)-(I-4), R³ is hydrogen, deuterium, halogen, —CN, —OH, —OR^{a}, —NR^{c}R^{d}, C₁-C₆alkyl, or C₁-C₆haloalkyl. In some embodiments of a compound of Formula (I), (Ia)-(Id), or (I-1)-(I-4), R³ is hydrogen, halogen, —OH, —OR^{a}, C₁-C₆alkyl, or C₁-C₆haloalkyl. In some embodiments of a compound of Formula (I), (Ia)-(Id), or (I-1)-(I-4), R³ is hydrogen, halogen, or C₁-C₆alkyl. In some embodiments of a compound of Formula (I), (Ia)-(Id), or (I-1)-(I-4), R³ is hydrogen or halogen. In some embodiments of a compound of Formula (I), (Ia)-(Id), or (I-1)-(I-4), R³ is hydrogen, halogen, or —OR^{a}. In some embodiments of a compound of Formula (I), (Ia)-(Id), or (I-1)-(I-4), R³ is halogen.

In some embodiments of a compound of Formula (I), (Ia)-(Id), or (I-1)-(I-4), R⁴ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (I), (Ia)-(Id), or (I-1)-(I-4), R⁴ is hydrogen, deuterium, halogen, —CN, —OH, —OR^{a}, —NR^{c}R^{d}, C₁-C₆alkyl, or C₁-C₆haloalkyl. In some embodiments of a compound of Formula (I), (Ia)-(Id), or (I-1)-(I-4), R⁴ is hydrogen, halogen, —OH, —OW, C₁-C₆alkyl, or C₁-C₆haloalkyl. In some embodiments of a compound of Formula (I), (Ia)-(Id), or (I-1)-(I-4), R⁴ is hydrogen, halogen, or C₁-C₆alkyl. In some embodiments of a compound of Formula (I), (Ia)-(Id), or (I-1)-(I-4), R⁴ is hydrogen or halogen. In some embodiments of a compound of Formula (I), (Ia)-(Id), or (I-1)-(I-4), R⁴ is hydrogen, halogen, or —OR^{a}. In some embodiments of a compound of Formula (I), (Ia)-(Id), or (I-1)-(I-4), R⁴ is halogen.

In some embodiments of a compound of Formula (I), (Ia)-(Id), or (I-1)-(I-4), R⁷ is deuterium, halogen, —CN, —NO₂, —OH, —OR^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl. In some embodiments of a compound of Formula (I), (Ia)-(Id), or (I-1)-(I-4), R⁷ is C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound of Formula (I), (Ia)-(Id), or (I-1)-(I-4), R⁷ is C₁-C₆alkyl, C₁-C₆haloalkyl, C₁-C₆deuteroalkyl, C₁-C₆hydroxyalkyl, or C₁-C₆aminoalkyl. In some embodiments of a compound of Formula (I), (Ia)-(Id), or (I-1)-(I-4), R⁷ is C₁-C₆alkyl or C₁-C₆haloalkyl. In some embodiments of a compound of Formula (I), (Ia)-(Id), or (I-1)-(I-4), R⁷ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound of Formula (I), (Ia)-(Id), or (I-1)-(I-4), R⁷ is hydrogen, deuterium, halogen, C₁-C₆alkyl, C₁-C₆haloalkyl, or C₁-C₆deuteroalkyl. In some embodiments of a compound of Formula (I), (Ia)-(Id), or (I-1)-(I-4), R⁷ is C₁-C₆alkyl or cycloalkyl. In some embodiments of a compound of Formula (I), (Ia)-(Id), or (I-1)-(I-4), R⁷ is C₁-C₆alkyl. In some embodiments of a compound of Formula (I), (Ia)-(Id), or (I-1)-(I-4), R⁷ is methyl.

In some embodiments of a compound of Formula (I), (Ia)-(Id), or (I-1)-(I-4), R⁸ is hydrogen or C₁-C₆alkyl. In some embodiments of a compound of Formula (I), (Ia)-(Id), or (I-1)-(I-4), R⁸ is hydrogen.

In some embodiments of a compound of Formula (I), (Ia)-(Id), or (I-1)-(I-4), Ring B is aryl or heteroaryl. In some embodiments of a compound of Formula (I), (Ia)-(Id), or (I-1)-(I-4), Ring B is phenyl. In some embodiments of a compound of Formula (I), (Ia)-(Id), or (I-1)-(I-4), Ring B is aryl or heteroaryl. In some embodiments of a compound of Formula (I), (Ia)-(Id), or (I-1)-(I-4), Ring B is 5- or 6-membered heteroaryl. In some embodiments of a compound of Formula (I), (Ia)-(Id), or (I-1)-(I-4), Ring B is 5-membered heteroaryl. In some embodiments of a compound of Formula (I), (Ia)-(Id), or (I-1)-(I-4), Ring B is 6-membered heteroaryl.

In some embodiments of a compound of Formula (I), (Ia)-(Id), or (I-1)-(I-4), each R⁹ is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R^{9a}; or two R⁹ on the same atom are taken together to form an oxo.

In some embodiments of a compound of Formula (I), (Ia)-(Id), or (I-1)-(I-4), each R⁹ is independently deuterium, halogen, —CN, —C(═O)NR^{c}R^{d}, C₁-C₆alkyl, C₁-C₆haloalkyl, C₁-C₆deuteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R^{9a}.

In some embodiments of a compound of Formula (I), (Ia)-(Id), or (I-1)-(I-4), each R⁹ is independently halogen or C₁-C₆alkyl.

In some embodiments of a compound of Formula (I), (Ia)-(Id), or (I-1)-(I-4), each R^{9a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkynyl, cycloalkyl, heterocycloalkyl, aryl, or heteroaryl; wherein the alkyl, alkynyl, cycloalkyl, heterocycloalkyl, aryl, and heteroaryl is optionally and independently substituted with one or more of deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

In some embodiments of a compound of Formula (I), (Ia)-(Id), or (I-1)-(I-4), each R^{9a} is independently deuterium, halogen, —OW, —C(═O)NR^{c}R^{d}, C₁-C₆alkyl, C₁-C₆haloalkyl, C₁-C₆deuteroalkyl, C₂-C₆alkynyl, cycloalkyl, or heterocycloalkyl; wherein the alkyl, alkynyl, cycloalkyl, and heterocycloalkyl is optionally and independently substituted with one or more of deuterium, halogen, —CN, —NO₂, —OH, -OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

In some embodiments of a compound of Formula (I), (Ia)-(Id), or (I-1)-(I-4), m is 1-3. In some embodiments of a compound of Formula (I), (Ia)-(Id), or (I-1)-(I-4), m is 0 or 1. In some embodiments of a compound of Formula (I), (Ia)-(Id), or (I-1)-(I-4), m is 1-3. In some embodiments of a compound of Formula (I), (Ia)-(Id), or (I-1)-(I-4), m is 0-2. In some embodiments of a compound of Formula (I), (Ia)-(Id), or (I-1)-(I-4), m is 1-3. In some embodiments of a compound of Formula (I), (Ia)-(Id), or (I-1)-(I-4), m is 1 or 2. In some embodiments of a compound of Formula (I), (Ia)-(Id), or (I-1)-(I-4), m is 0-3. In some embodiments of a compound of Formula (I), (Ia)-(Id), or (I-1)-(I-4), m is 1. In some embodiments of a compound of Formula (I), (Ia)-(Id), or (I-1)-(I-4), m is 2. In some embodiments of a compound of Formula (I), (Ia)-(Id), or (I-1)-(I-4), m is 3. In some embodiments of a compound of Formula (I), (Ia)-(Id), or (I-1)-(I-4), m is 4.

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound disclosed herein, each R^{a} is independently C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is independently C₁-C₆alkyl. In some embodiments of a compound disclosed herein, each R^{a} is hydrogen.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R and R^{a} are independently hydrogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are independently C₁-C₆alkyl. In some embodiments of a compound disclosed herein, each R^{c} and R^{d} are hydrogen.

In some embodiments of a compound disclosed herein, R^{c} and R^{d} are taken together with the atom to which they are attached to form a heterocycloalkyl optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, R and R^{a} are taken together with the atom to which they are attached to form a heterocycloalkyl optionally substituted with one or more substituents that is oxo, —S(═O)CH₃, —S(═O)₂CH₃, —S(═O)₂NH₂, —S(═O)₂NHCH₃, —S(═O)₂N(CH₃)₂, C₁-C₆alkyl, C₁-C₆haloalkyl, C₁-C₆deuteroalkyl, C₁-C₆hydroxyalkyl, or C₁-C₆aminoalkyl.

In some embodiments of a compound disclosed herein, each R⁹, R^{9a}, R^{a}, R^{b}, R^{c}, R^{d}, and the heterocycloalkyl formed when R^{c} and R^{d} are taken together, is independently substituted with one, two, three, or four substituents as defined herein. In some embodiments of a compound disclosed herein, R⁹, R^{9a}, R^{a}, R^{b}, R, R, and the heterocycloalkyl formed when R^{c} and R^{d} are taken together, is independently substituted with one, two, or three substituents as defined herein. In some embodiments of a compound disclosed herein, R⁹, R^{9a}, R^{a}, R^{b}, R^{c}, R^{d}, and the heterocycloalkyl formed when R^{c} and R^{d} are taken together, is independently substituted with one or two substituents as defined herein. In some embodiments of a compound disclosed herein, R⁹, R^{9a}, R^{a}, R^{b}, R^{c}, R^{d}, and the heterocycloalkyl formed when R^{c} and R^{d} are taken together, is independently substituted with one substituent as defined herein. In some embodiments of a compound disclosed herein, R⁹, R^{9a}, R^{a}, R^{b}, R^{c}, R^{d}, and the heterocycloalkyl formed when R^{c} and R^{d} are taken together, is independently substituted with two substituents as defined herein. In some embodiments of a compound disclosed herein, R⁹, R^{9a}, R^{a}, R^{b}, R^{c}, R^{d}, and the heterocycloalkyl formed when R^{c} and R^{d} are taken together, is independently substituted with three substituents as defined herein.

In some embodiments of a compound of Formula (I) or (Ia)-(Id), the compound is selected from a compound of Table 1:

TABLE 1

| Ex. | Name | Structure |
|---|---|---|
| 1 | 5-((1S,2R)-1- (5-chloro-1,1- dioxido-3- oxobenzo[d] isothiazol-2(3H)- yl)-2-(6-fluoro- 2,3- dimethylphenyl) propyl)-1,3,4- oxadiazol-2(3H)- one | 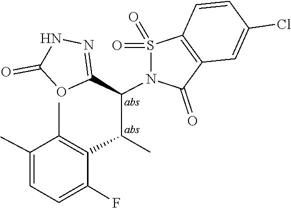 [SMILES: Cc1ccc(F)c([C@@H](C)[C@@H](c2n[nH]c(=O)o2)N2C(=O)c3cc(Cl)ccc3S2(=O)=O)c1C] |
| 2 | 5-((1S,2S)-1- (5-chloro-1,1- dioxido-3- oxobenzo[d] isothiazol-2(3H)- yl)-2-(6- fluoro-2,3- dimethylphenyl) propyl)-1,3,4- oxadiazol-2(3H)- one | 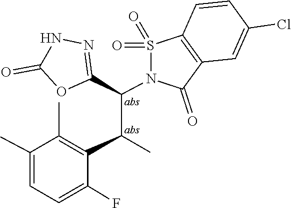 [SMILES: Cc1ccc(F)c([C@H](C)[C@@H](c2n[nH]c(=O)o2)N2C(=O)c3cc(Cl)ccc3S2(=O)=O)c1C] |
| 3 | 5-((1R,2S)-1- (5-chloro-7- methoxy-1,1- dioxido-3- oxobenzo[d] isothiazol- 2(3H)- yl)-2-(6- fluoro-2,3- dimethylphenyl) propyl)-1,3,4- oxadiazol- 2(3H)-one | 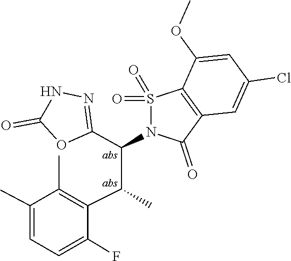 [SMILES: COc1cc(Cl)cc2c1S(=O)(=O)N([C@H](c1n[nH]c(=O)o1)[C@H](C)c1c(F)ccc(C)c1C)C2=O] |
| 4 | 5-((1R,2R)-1- (5-chloro-7- methoxy-1,1- dioxido-3- oxobenzo[d] isothiazol- 2(3H)- yl)-2-(6- fluoro-2,3- dimethylphenyl) propyl)-1,3,4- oxadiazol-2(3H)- one | 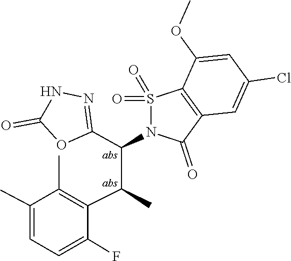 [SMILES: COc1cc(Cl)cc2c1S(=O)(=O)N([C@H](c1n[nH]c(=O)o1)[C@@H](C)c1c(F)ccc(C)c1C)C2=O] |
| 5 | 5-((1S,2S)-1- (5-chloro-7- methoxy-1,1- dioxidobenzo [d]isothiazol- 2(3H)-yl)-2- (6-fluoro-2,3- dimethylphenyl) propyl)-1,3,4- oxadiazol- 2(3H)-one | 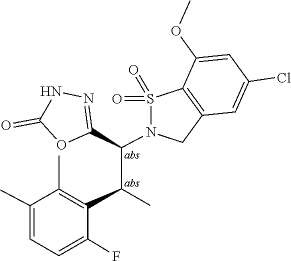 [SMILES: COc1cc(Cl)cc2c1S(=O)(=O)N([C@H](c1n[nH]c(=O)o1)[C@@H](C)c1c(F)ccc(C)c1C)C2] |
| 6 | 5-((1S,2R)-1- (5-chloro-7- methoxy-1,1- dioxidobenzo [d]isothiazol- 2(3H)-yl)-2- (6-fluoro-2,3- dimethylphenyl) propyl)-1,3,4- oxadiazol- 2(3H)-one | 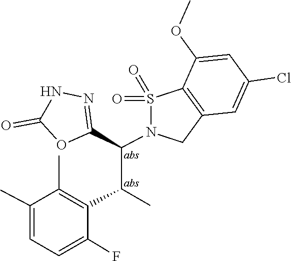 [SMILES: COc1cc(Cl)cc2c1S(=O)(=O)N([C@H](c1n[nH]c(=O)o1)[C@H](C)c1c(F)ccc(C)c1C)C2] |
| 7 | 5-((1S,2R)-1- (6-chloro-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 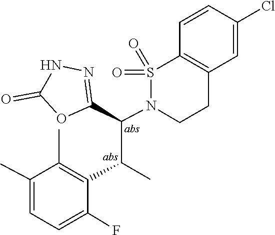 [SMILES: Cc1ccc(F)c([C@@H](C)[C@@H](c2n[nH]c(=O)o2)N2CCc3cc(Cl)ccc3S2(=O)=O)c1C] |
| 8 | 5-((1R,2S)-1- (6-chloro-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 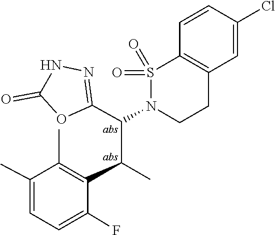 |
| 9 | 5-((1S,2R)-1- (7-chloro-1,1- dioxido-4,5- dihydrobenzo [f][1,2] thiazepin- 2(3H)-yl)-2- (6-fluoro-2,3- dimethylphenyl) propyl)-1,3,4- oxadiazol- 2(3H)-one | 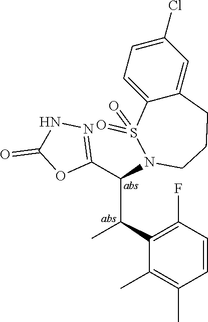 [SMILES: Cc1ccc(F)c([C@@H](C)[C@@H](c2n[nH]c(=O)o2)N2CCCc3cc(Cl)ccc3S2(=O)=O)c1C] |
| 10 | 5-((1S,2S)-1- (7-chloro-1,1- dioxido-4,5- dihydrobenzo [f][1,2]thiazepin- 2(3H)-yl)-2- (6-fluoro-2,3- dimethylphenyl) propyl)-1,3,4- oxadiazol- 2(3H)-one | 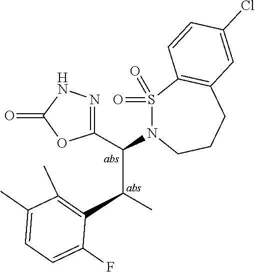 [SMILES: Cc1ccc(F)c([C@H](C)[C@@H](c2n[nH]c(=O)o2)N2CCCc3cc(Cl)ccc3S2(=O)=O)c1C] |
| 11 | 5-((1S,2R)-1- (7-chloro-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 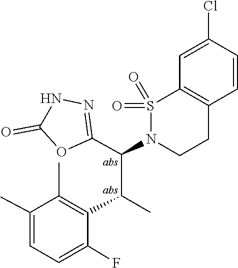 [SMILES: Cc1ccc(F)c([C@@H](C)[C@@H](c2n[nH]c(=O)o2)N2CCc3ccc(Cl)cc3S2(=O)=O)c1C] |
| 12 | 5-((1S,2s)-1- (7-chloro-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 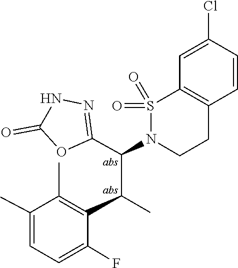 [SMILES: Cc1ccc(F)c([C@H](C)[C@@H](c2n[nH]c(=O)o2)N2CCc3ccc(Cl)cc3S2(=O)=O)c1C] |
| 13 | 6-chloro-2- ((1S,2R)-2-(6- fluoro-2,3- dimethylphenyl)- 1-(2H-tetrazol- 5-yl)propyl)- 3,4- dihydro-2H- benzo[e][1,2] thiazine 1,1- dioxide | 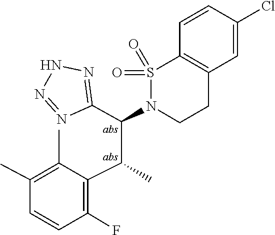 [SMILES: Cc1ccc(F)c([C@@H](C)[C@@H](c2nn[nH]n2)N2CCc3cc(Cl)ccc3S2(=O)=O)c1C] |
| 14 | 5-((1S,2R)-1- (5-chloro-1,1- dioxidobenzo [d]isothiazol- 2(3H)-yl)-2- (6-fluoro-2,3- dimethylphenyl) propyl)-1,3,4- oxadiazol- 2(3H)-one | 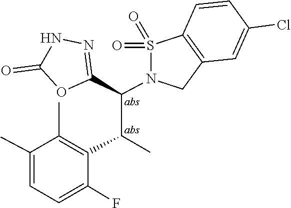 [SMILES: Cc1ccc(F)c([C@@H](C)[C@@H](c2n[nH]c(=O)o2)N2Cc3cc(Cl)ccc3S2(=O)=O)c1C] |
| 15 | 5-((1R,2S)-1- (5-chloro-7- methoxy-1,1- dioxido-3- oxobenzo[d] isothiazol- 2(3H)- yl)-2-(6- fluoro-2,3- dimethylphenyl) propyl)-1,3,4- oxadiazol- 2(3H)-one | 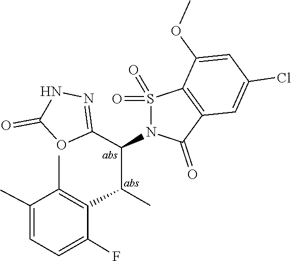 [SMILES: COc1cc(Cl)cc2c1S(=O)(=O)N([C@H](c1n[nH]c(=O)o1)[C@H](C)c1c(F)ccc(C)c1C)C2=O] |
| 16 | 5-((1S)-1-(6- chloro-1,1- dioxido-4-oxo- 3,4-dihydro- 2H-benzo[e] [1,2]thiazin- 2-yl)-2-(6- fluoro-2,3- dimethylphenyl) propyl)-1,3,4- oxadiazol- 2(3H)-one | 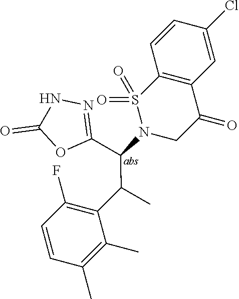 [SMILES: Cc1ccc(F)c(C(C)[C@@H](c2n[nH]c(=O)o2)N2CC(=O)c3cc(Cl)ccc3S2(=O)=O)c1C] |
| 17 | 5-((1S)-1-(6- chloro-1,1- dioxido-3-oxo- 3,4-dihydro- 2H-benzo[e] [1,2]thiazin- 2-yl)-2-(6- fluoro-2,3- dimethylphenyl) propyl)-1,3,4- oxadiazol- 2(3H)-one | 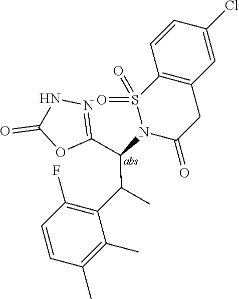 [SMILES: Cc1ccc(F)c(C(C)[C@@H](c2n[nH]c(=O)o2)N2C(=O)Cc3cc(Cl)ccc3S2(=O)=O)c1C] |
| 18 | 5-((1S,2R)-1- (1,1-dioxido- 3,4-dihydro- 2H-benzo[e] [1,2]thiazin- 2-yl)-2- (6-fluoro-2,3- dimethylphenyl) propyl)-1,3,4- oxadiazol- 2(3H)-one | 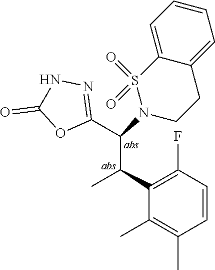 [SMILES: Cc1ccc(F)c([C@@H](C)[C@@H](c2n[nH]c(=O)o2)N2CCc3ccccc3S2(=O)=O)c1C] |
| 19 | 5-((1S,2R)-1- (6-chloro-4- hydroxy-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 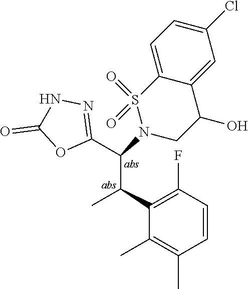 [SMILES: Cc1ccc(F)c([C@@H](C)[C@@H](c2n[nH]c(=O)o2)N2CC(O)c3cc(Cl)ccc3S2(=O)=O)c1C] |
| 20 | 5-((1S)-1-(6- chloro-4- methyl-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 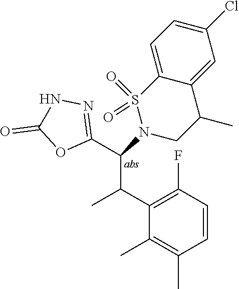 [SMILES: Cc1ccc(F)c(C(C)[C@@H](c2n[nH]c(=O)o2)N2CC(C)c3cc(Cl)ccc3S2(=O)=O)c1C] |
| 21 | 5-((1S)-1-(6- chloro-4,4- dimethyl-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 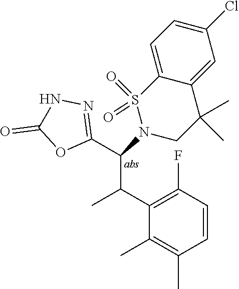 [SMILES: Cc1ccc(F)c(C(C)[C@@H](c2n[nH]c(=O)o2)N2CC(C)(C)c3cc(Cl)ccc3S2(=O)=O)c1C] |
| 22 | 5-((1S,2R)-1- (6-chloro-4,4- difluoro-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 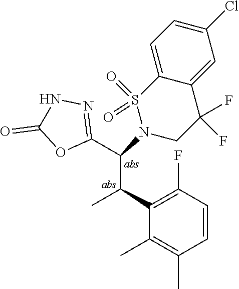 [SMILES: Cc1ccc(F)c([C@@H](C)[C@@H](c2n[nH]c(=O)o2)N2CC(F)(F)c3cc(Cl)ccc3S2(=O)=O)c1C] |
| 23 | 5-((1S,2R)-1- (5-chloro-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 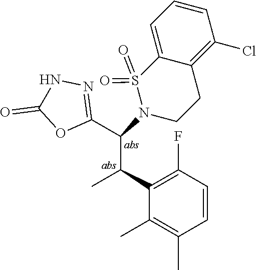 [SMILES: Cc1ccc(F)c([C@@H](C)[C@@H](c2n[nH]c(=O)o2)N2CCc3c(Cl)cccc3S2(=O)=O)c1C] |
| 24 | 5-((1S,2R)-1- (6-chloro-1,1- dioxido-3,4- dihydro-2H- pyrido[2,3-e] [1,2]thiazin-2- yl)-2-(6- fluoro-2,3- dimethylphenyl) propyl)-1,3,4- oxadiazol- 2(3H)-one | 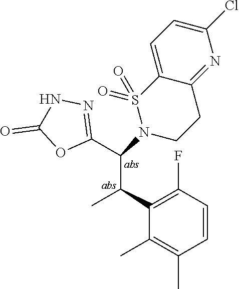 [SMILES: Cc1ccc(F)c([C@@H](C)[C@@H](c2n[nH]c(=O)o2)N2CCc3nc(Cl)ccc3S2(=O)=O)c1C] |
| 25 | 5-((1S,2R)-1- (6-chloro-4- hydroxy-4- methyl-1,1- dioxido-3,4- dihydro-2H- benzo[e][1,2] thiazin-2-yl)-2- (6-fluoro-2,3- dimethylphenyl) propyl)-1,3,4- oxadiazol- 2(3H)-one | 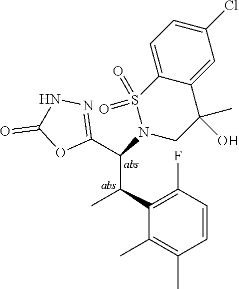 [SMILES: Cc1ccc(F)c([C@@H](C)[C@@H](c2n[nH]c(=O)o2)N2CC(C)(O)c3cc(Cl)ccc3S2(=O)=O)c1C] |
| 26 | 5-((1S)-1-((4S, 5R)-7-chloro- 4,5-dihydroxy- 1,1-dioxido- 4,5- dihydrobenzo [f][1,2] thiazepin- 2(3H)-yl)-2- (6-fluoro-2,3- dimethylphenyl) propyl)-1,3,4- oxadiazol- 2(3H)-one | 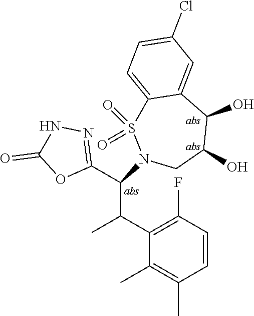 [SMILES: Cc1ccc(F)c(C(C)[C@@H](c2n[nH]c(=O)o2)N2C[C@H](O)[C@H](O)c3cc(Cl)ccc3S2(=O)=O)c1C] |
| 27 | 5-((1S,2R)-1- (6-chloro-1,1- dioxido-3,4- dihydro-2H- pyrido[4,3-e] [1,2]thiazin-2- yl)-2-(6- fluoro-2,3- dimethylphenyl) propyl)-1,3,4- oxadiazol- 2(3H)-one | 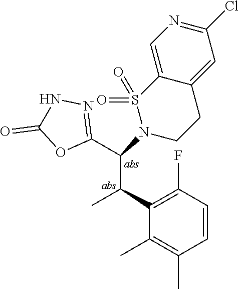 [SMILES: Cc1ccc(F)c([C@@H](C)[C@@H](c2n[nH]c(=O)o2)N2CCc3cc(Cl)ncc3S2(=O)=O)c1C] |

The absolute label (abs) is added to a chiral center to denote that it is unambiguously a pure sample of the drawn stereoisomer.

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, tautomer, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²p, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, tautomer, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like. Representative salts include the alkali or alkaline earth salts, like lithium, sodium, potassium, calcium, and magnesium, and aluminum salts and the like of the tetrazole.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. In some embodiments, the tetrazoles disclosed herein exists as either of its tautomers:

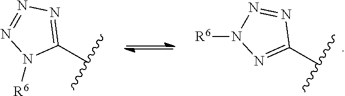

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modem Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising a compound described herein, or a pharmaceutically acceptable salt, solvate, tautomer, or stereoisomer thereof, and a pharmaceutically acceptable excipient.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal, and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a solution, an emulsion, an ointment, a lotion, an eye drop, or an ear drop. In some embodiments, the pharmaceutical composition is formulated as a tablet.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involves the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

### Methods of Treatment

Disclosed herein are methods for treating cancer in a subject in need thereof, including administering to the subject a therapeutically effective amount of a compound disclosed herein, or a pharmaceutically acceptable salt, solvate, tautomer, or stereoisomer thereof. Disclosed herein are methods for treating a RNR-related cancer in a subject in need thereof, including administering to the subject a therapeutically effective amount of a compound disclosed herein, or a pharmaceutically acceptable salt, solvate, tautomer, or stereoisomer thereof.

In some embodiments, the RNR-related cancer includes malignant tumors whose incidence can be decreased or whose symptom is in remission or alleviated and/or completely cured by deleting or suppressing and/or inhibiting functions of RNR. Malignant tumors of interest is, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), etc.), lung cancer (non-small cell lung cancer, small cell lung cancer, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors, skin cancer, brain tumor and the like.

In some embodiments, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, tautomer, or stereoisomer thereof, pharmaceutical compositions include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, Herceptin (trastuzumab) resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus, Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, lobular carcinoma, ductal carcinoma, cancer of the pancreatic stellate cells, cancer of the hepatic stellate cells, or prostate cancer. In embodiments, the cancer is selected from ovarian cancer, prostate cancer, esophageal cancer, salivary gland cancer, breast cancer, liver cancer, pancreatic cancer, stomach cancer, lung cancer, bladder cancer, colon cancer, and uterine cancer. In embodiments, the cancer is selected from muscle cancer, brain cancer, lymph node cancer, thyroid cancer, kidney cancer, and adrenal gland cancer.

ecDNA mediates an important and clinically distinct mechanism of resistance to targeted therapies. There are immediate therapeutic opportunities for utility of the one or more RNR inhibitor described herein as a single agent or in combination with other therapies. In some embodiments, the one or more RNR inhibitor described herein may be used to treat an ecDNA+cancer, ecDNA+tumor or ecDNA+tumor cells. One or more RNR inhibitor described herein may be used to treat tumors, such as with one or more amplified oncogenes (e.g. FGFR, EGFR, MET, KRAS, MDM2 amplifications), in some cases, the one or more amplified oncogenes comprise non-mutant forms of the oncogene and in some cases, the amplified oncogenes comprises mutant forms of the oncogenes. In some cases, the tumor comprises one or more amplified oncogenes present on ecDNA and the one or more RNR inhibitor described herein are used to treat the tumor in combination with a therapeutic agent targeted to (e.g., an inhibitor of) the one or more amplified oncogenes on the ecDNA. One or more RNR inhibitor described herein may be used to treat tumors for which there are no approved targeted therapies or for which highly efficacious therapies are lacking. One or more RNR inhibitor described herein may be used to treat tumors that have developed resistance to another therapy such as a resistance to a targeted agent. In some cases, a tumor (or tumor cells) treated with one or more targeted agents develops resistance to a targeted agent, such as a targeted agent directed to an oncogene or a targeted agent that directly inhibits activating mutant forms of certain oncoproteins (e.g. KRAS, BRAF, EGFR) or as a consequence of focal amplification such as ecDNA-based amplification of the target gene itself, and the one or more RNR inhibitor described herein may be used to treat such tumors or tumor cells, alone or in combination with an additional therapeutic agent.

Provided herein are methods wherein inhibition of RNR by the one or more RNR inhibitors described herein exhibits synthetic lethality with a cancer-targeted agent. In some embodiments, synthetic lethality arises with one or more RNR inhibitors described herein in combination with a cancer targeted agent. In some cases, a tumor background is identified as hyper-sensitive to a RNR inhibitor and allows a sufficient therapeutic index to enable tolerated doses that are efficacious. In some embodiments, synthetic lethality arises with one or more RNR inhibitors described herein in combination with a cancer targeted agent where the tumor or tumor cells are ecDNA+. In some cases, RNR inhibition results in reduced ecDNA copy number. In some cases, RNR inhibition results in enhanced cytotoxicity in ecDNA+cells. In some cases, enhanced cytotoxicity results from the combination of RNR inhibition and inhibition of a cancer-target, such as an oncogene, for example an oncogene amplified on ecDNA.

In an aspect of methods herein, a tumor or tumor cells to be treated are ecDNA+. In some cases, such tumor or tumor cells are determined to have an ecDNA signature. In some cases, a tumor or tumor cells are determined to have an ecDNA signature when the tumor or tumor cells have one or more characteristics associated with ecDNA+tumors or tumor cells. For example, in some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof.

### Combination Therapy

In certain instances, the compound described herein, or a pharmaceutically acceptable salt, solvate, tautomer, or stereoisomer thereof, is administered in combination with a second therapeutic agent or a cancer-targeted agent.

In an aspect of methods herein, the method further comprises administering a cancer-targeted therapeutic agent, directed to an activity of a protein product of a target gene. In some cases, the treatment with the cancer-targeted therapeutic agent and the RNR inhibitor disclosed herein reduces amplification or expression of the target gene in the tumor or tumor cells. In some cases, the cancer-targeted therapeutic agent is administered prior to the RNR inhibitor. In some cases, the cancer-targeted therapeutic agent is administered concurrently with the RNR inhibitor.

In an aspect of methods herein, the tumor or tumor cells have an ecDNA signature. In some cases, the tumor or tumor cells develop the ecDNA signature after administration of the cancer-targeted therapeutic agent. In some cases, the tumor or tumor cells develop the ecDNA signature prior to treatment. In some cases, the method prevents an increase of ecDNA in the tumor or tumor cells.

In some embodiment, the second therapeutic includes antimetabolites, platinum drugs, plant alkaloid drugs, and molecular targeting drugs.

In some embodiments, the antimetabolites include 5-fluorouracil, 5-fluoro-2′-deoxyuridine, tegafur, tegafur-uracil, tegafur-gimeracil-oteracil, pemetrexed, trifluridine, trifluridine-tipiracil hydrochloride, fludarabine (or an active metabolite fludarabine nucleoside), cytarabine, gemcitabine, capecitabine, nelarabine, clofarabine, and DNA methylation inhibitors (decitabine, guadecitabine, azacitidine, etc.).

In some embodiments, the platinum drugs include cisplatin, oxaliplatin, carboplatin, and nedaplatin.

In some embodiments, the plant alkaloid drugs include microtube inhibiting drugs such as paclitaxel, docetaxel, vinblastine, vincristine, vindesine, vinorelbine, and eribulin, and topoisomerase inhibiting drugs such as irinotecan (or an active metabolite SN-38), nogitecan, and etoposide.

In some embodiments, the molecular targeting drugs include ATR (ataxia telangiectasia and Rad3 related protein) inhibitors, Chk1 (checkpoint kinase 1) inhibitors, HSP (heat shock protein) 90 inhibitors, PARP (poly ADP ribose polymerase) inhibitors, EGFR (epidermal growth factor receptor) inhibitors, Her2 inhibitors, VEGFR (vascular endothelial growth factor receptor) inhibitors, PDGFR (platelet-derived growth factor receptor) inhibitors, MET inhibitors, AXL inhibitors, RET inhibitors, FLT3 (fis-related tyrosine kinase 3) inhibitors, KIT inhibitors, CSF1R (colony-stimulating factor 1 receptor) inhibitors, TIE2 (tunica interna endothelial cell kinase 2) inhibitors, TRKB inhibitors, and CDK4/6 inhibitors. In some embodiments, the ATR inhibitors include AZD6738, berzosertib, BAY1895344, and VX-803. In some embodiments, the Chk1 inhibitors include prexasertib, SCH900776, GDC-0575, and CCT245737. In some embodiments, the HSP90 inhibitors include luminespib, ganetespib, and onalespib. In some embodiments, the PARP inhibitors include olaparib, rucaparib, niraparib, veliparib, and talazoparib. In some embodiments, the EGFR inhibitors include small molecule inhibitors such as lapatinib, gefitinib, erlotinib, afatinib, and vandetanib, and anti-EGFR antibodies such as cetuximab and panitumumab. In some embodiments, the Her2 inhibitors include small molecule inhibitors such as lapatinib, and anti-Her2 antibodies such as trastuzumab, pertuzumab, and trastuzumab emtansine. In some embodiments, the VEGFR inhibitors are inhibitors of at least one of VEGFR1, VEGFR2, and VEGFR3 and include small molecule inhibitors such as sunitinib, cabozantinib, midostaurin, sorafenib, vandetanib, pazopanib, lenvatinib, and axitinib, and anti-VEGFR antibodies such as ramucirumab. In some embodiments, the PDGFR inhibitors are PDGFRa and/or PDGFRO inhibitors and include sunitinib, midostaurin, pazopanib, lenvatinib, and sorafenib. In some embodiments, the MET inhibitors include cabozantinib, crizotinib, and tepotinib. In some embodiments, the AXL inhibitors include cabozantinib and gilteritinib. In some embodiments, the RET inhibitors include sunitinib, cabozantinib, sorafenib, lenvatinib, and vandetanib. In some embodiments, the FLT3 inhibitors include sunitinib, cabozantinib, midostaurin, gilteritinib, and sorafenib. In some embodiments, the KIT inhibitors include sunitinib, midostaurin, pazopanib, lenvatinib, and sorafenib. In some embodiments, the CSF1R inhibitors include sunitinib, BLZ-945, and ARRY-382. In some embodiments, the TIE2 inhibitors include cabozantinib. In some embodiments, the TRKB inhibitors include cabozantinib and entrectinib. In some embodiments, the CDK4/6 inhibitors include palbociclib, ribociclib, and abemaciclib.

In some embodiments, the benefit experienced by a patient is increased by administering one of the compounds described herein with a second therapeutic agent (which also includes a therapeutic regimen) that also has therapeutic benefit.

In one specific embodiment, a compound described herein, or a pharmaceutically acceptable salt, solvate, tautomer, or stereoisomer thereof, is co-administered with a second therapeutic agent, wherein the compound described herein, or a pharmaceutically acceptable salt, solvate, tautomer, or stereoisomer thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of the compounds disclosed herein will be utilized in formulating a pharmaceutical composition and/or in treatment regimens when the compounds disclosed herein are administered in combination with a second therapeutic agent. Therapeutically-effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt, solvate, tautomer, or stereoisomer thereof, is initiated prior to, during, or after treatment with a second agent described herein, and continues until any time during treatment with the second agent or after termination of treatment with the second agent. It also includes treatments in which a compound described herein, or a pharmaceutically acceptable salt, solvate, tautomer, or stereoisomer thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt, solvate, tautomer, or stereoisomer thereof, as well as combination therapies, are administered before, during, or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, the compound of described herein, or a pharmaceutically acceptable salt, solvate, tautomer, or stereoisomer thereof, is administered in combination with an adjuvant. In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced).

### EXAMPLES

### Synthesis of the Common Intermediate I: 5-((1S)-1-amino-2-(6-fluoro-2,3-dimethylphenyl) propyl)-1,3,4-oxadiazol-2(3H)-one

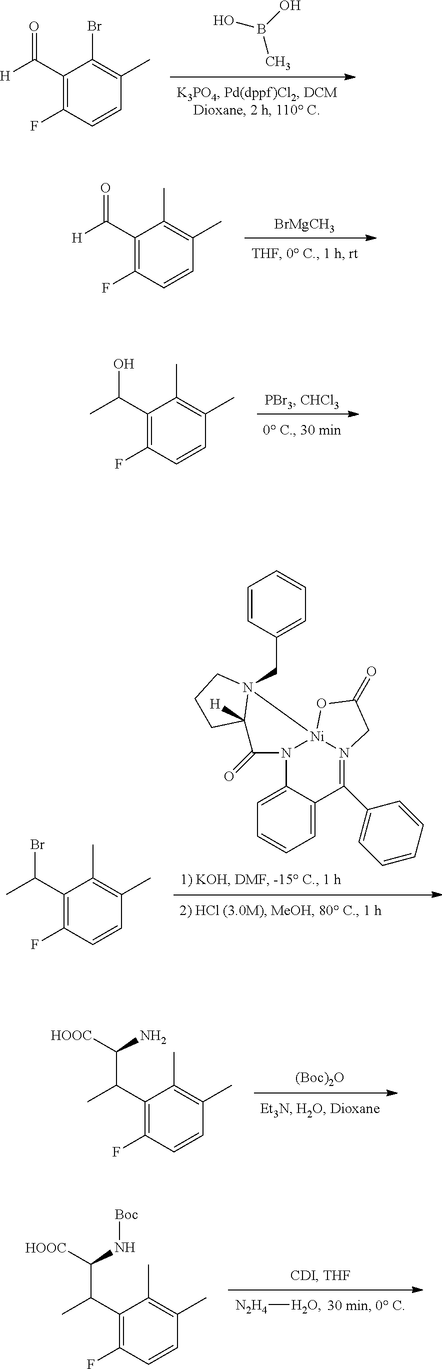

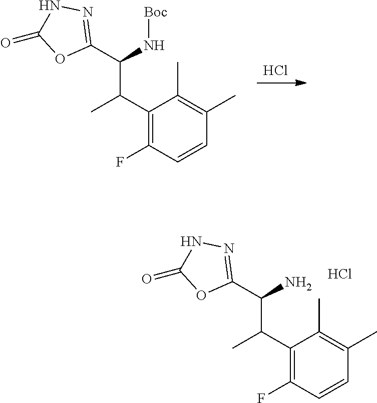 [SMILES: Cc1ccc(F)c(C(C)[C@H](NC(=O)OC(C)(C)C)c2n[nH]c(=O)o2)c1C ; Cl ; Cc1ccc(F)c(C(C)[C@H](N)c2n[nH]c(=O)o2)c1C ; Cl]

### Step 1. Synthesis of 6-fluoro-2,3-dimethylbenzaldehyde

Into a 1 L round-bottom flask purged and maintained with an inert atmosphere of nitrogen, was placed 2-bromo-6-fluoro-3-methylbenzaldehyde (50 g, 230 mmol, 1.0 equiv), methylboronic acid (23.4 g, 392 mmol, 1.7equiv), K₃PO₄ (117.4 g, 553 mmol, 2.4 equiv), Pd(dppf)Cl₂·CH₂Cl₂ (5.63 g, 6.91 mmol, 0.03equiv), H₂O (50 mL), Dioxane (450 mL). The resulting solution was stirred 2 hr at 110 degrees C. The reaction was then quenched by the addition of 200 mL of brine. The resulting solution was extracted with 3×50 mL of ethyl acetate, and the organic layers combined. The residue was applied onto a silica gel column with ethyl acetate/petroleum ether (1:3). This resulted in 6-fluoro-2,3-dimethylbenzaldehyde (30 g, 85%).

### Step 2. 1-(6-fluoro-2,3-dimethylphenyl) ethan-1-ol

Into a 1 L 3-necked round-bottom flask, the mixture of 6-fluoro-2,3-dimethylbenzaldehyde (27 g, 177.4 mmol, 1 equiv) in THF was added bromo(methyl)magnesium (42.3 g, 355 mmol, 2 equiv) dropwise at 0 degrees C. under nitrogen atmosphere. The resulting mixture was stirred for 2 h at room temperature. The reaction was quenched with sat. NH₄Cl (aq.) at room temperature. The resulting mixture was extracted with EtOAc (3×50 mL). The combined organic layers were washed with brine (2×20 mL), dried over anhydrous MgSO₄. After filtration, the filtrate was concentrated under reduced pressure. to afford 1-(6-fluoro-2,3-dimethylphenyl) ethanol (27 g, 90.5%).

### Step 3. 2-(1-bromoethyl)-1-fluoro-3,4-dimethylbenzene

Into a 500 mL 3-necked round-bottom flask were added 1-(6-fluoro-2,3-dimethylphenyl) ethanol (25 g, 148.6 mmol, 1.0 equiv) and CHCl₃ (250 mL) at room temperature. To the above mixture was added PBr3 (63.5 mL, 668.8 mmol, 4.5equiv) dropwise at 0 degrees C. The resulting mixture was stirred for additional 30 min at 0 degrees C. The reaction was quenched by the addition of NaHCO₃ (aq.) (100 mL) at 0 degrees C. The resulting mixture was extracted with CH₂Cl₂ (3×50 mL). The combined organic layers were washed with brine (1×30 mL), dried over anhydrous MgSO₄. After filtration, the filtrate was concentrated under reduced pressure to afford 2-(1-bromoethyl)-1-fluoro-3,4-dimethylbenzene (29 g, 84.4%).

### Step 4. (2S)-2-amino-3-(6-fluoro-2,3-dimethylphenyl) butanoic acid

Into a 500 mL 3-necked round-bottom flask, to a mixture of Ni—(S)—BPB-Gly (5.39 g, 10.8 mmol, 0.5equiv) in DMF (42.4 mL) was added 2-(1-bromoethyl)-1-fluoro-3,4-dimethylbenzene (5 g, 21.6 mmol, 1.0 equiv) dropwise at room temperature under nitrogen atmosphere. To the resulting mixture was added KOH (6.07 g, 108.2 mmol, 5.0 equiv) in portions at −15 degrees C. under nitrogen atmosphere, stirred for 1 h at −15 degrees C. under nitrogen atmosphere. The reaction was quenched by the addition of sat. NH₄Cl (aq.) (100 mL) at room temperature, extracted with EtOAc (3×30 mL). The combined organic layers were washed with brine (3×30 mL), dried over anhydrous Na₂SO₄. After filtration, the filtrate was concentrated under reduced pressure, purified by silica gel column chromatography. To the mixture were added MeOH (42 mL) and HCl (50 mL) at room temperature. The resulting mixture was stirred for 1 h at 80 degrees C., then purified by reverse phase flash to afford (2S)-2-amino-3-(6-fluoro-2,3-dimethylphenyl)butanoic acid (3.05 g, 63%).

### Step 5. (2S)-2-((tert-butoxycarbonyl) amino)-3-(6-fluoro-2,3-dimethylphenyl) butanoic acid

Into a 250 mL round-bottom flask were added (2S)-2-amino-3-(6-fluoro-2,3-dimethylphenyl) butanoic acid (5 g, 22.2 mmol, 1.0 equiv), Et₃N (6.74 g, 66.6 mmol, 3.0 equiv), H₂O (25 mL) and Dioxane (25 mL) at room temperature. To the resulting mixture was added di-tert-butyl dicarbonate (7.27 g, 33.3 mmol, 1.5 equiv) in portions at 0 degrees C. The resulting mixture was stirred for 2 h at room temperature. The crude product was purified by reverse phase flash chromatography to afford (2S)-2-[(tert-butoxycarbonyl) amino]-3-(6-fluoro-2,3-dimethylphenyl) butanoic acid (3.5 g, 48.5%).

### Step 6: synthesis of 5-((1S)-1-amino-2-(6-fluoro-2,3-dimethylphenyl) propyl)-1,3,4-oxadiazol-2(3H)-one, HCl

Into a 250-mL round-bottom flask were added (2S)-2-[(tert-butoxycarbonyl) amino]-3-(6-fluoro-2,3-dimethylphenyl) butanoic acid (9 g, 27.7 mmol, 1.0 equiv), CDI (11.2 g, 69.2 mmol, 2.5equiv) and THF (60 mL) at room temperature, the resulting mixture was stirred for 30 min at room temperature. To the mixture was added hydrazine (4.15 mL, 82.901 mmol, 3.0 equiv) dropwise at 0 degrees C. The resulting mixture was stirred for 30 min at 0 degrees C. The reaction was quenched with water at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure. To the crude mixture was added dioxane (60 mL) and CDI (11.2 g, 69.2 mmol, 2.5equiv) at room temperature. The resulting mixture was stirred for 30 min at room temperature. The resulting mixture was extracted with EtOAc (3×20 mL). The combined organic layers were washed with brine (2×10 mL), dried over anhydrous Na₂SO₄. After filtration, the filtrate was concentrated under reduced pressure, purified by silica gel column chromatography to afford tert-butyl N-[(1S,2R)-2-(6-fluoro-2,3-dimethylphenyl)-1-(5-oxo-4H-1,3,4-oxadiazol-2-yl) propyl]carbamate (3 g, 29.7%).

The product was dissolved in 2 ml THF and treated with 2 ml of HCl 4N in THF. The reaction was left overnight at RT and concentrated under vacuum yielding 5-((1S)-1-amino-2-(6-fluoro-2,3-dimethylphenyl) propyl)-1,3,4-oxadiazol-2(3H)-one, HCl (2.4 g, Yield 100%).

### Synthesis of the Common Intermediate II: methyl (2S)-2-amino-3-(6-fluoro-2,3-dimethylphenyl) butanoate

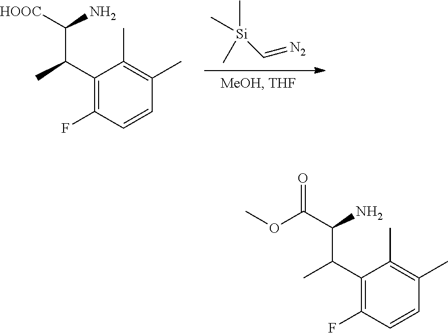

Into a 500 mL 3-necked round-bottom flask were added (2S)-2-amino-3-(6-fluoro-2,3-dimethylphenyl) butanoic acid (12.8 g, 56.8 mmol, 1.00 equiv), trimethylsilyldiazomethane (56.8 mL, 113.6 mmol, 2.0 equiv), MeOH (130 mL) and THF (380 mL) at room temperature. The resulting mixture was stirred for 3 h at room temperature under nitrogen atmosphere. The resulting mixture was concentrated with to afford methyl (2S)-2-amino-3-(6-fluoro-2,3-dimethylphenyl) butanoate (9.9 g, 72.8%).

### Synthesis of the Common Intermediate III: tert-butyl (2S)-2-amino-3-(6-fluoro-2,3-dimethylphenyl) butanoate

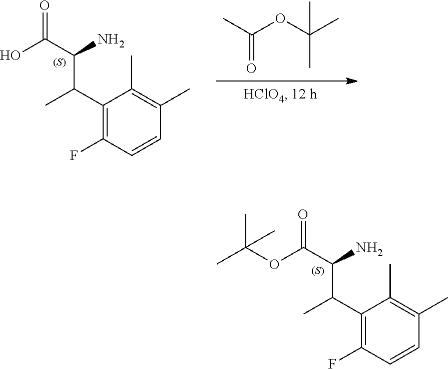

Into a 250 mL round-bottom flask were added (2S)-2-amino-3-(6-fluoro-2,3-dimethylphenyl) butanoic acid (15 g, 67 mmol, 1 equiv) and tert-butyl acetate (160 mL) at room temperature. To the above mixture was added HClO₄ (21 mL, 366 mmol, 5.50 equiv) dropwise at 0° C. The resulting mixture was stirred for additional 1 h at room temperature. The reaction was quenched by the addition of HCl (1M) (240 mL) at room temperature. The mixture was basified to pH9 with Na₂CO₃ (solid) (300 mL). The resulting mixture was extracted with EtOAc (3×300 mL). The combined organic layers were washed with brine (1×300 mL), dried over anhydrous Na₂SO₄. After filtration, the filtrate was concentrated under reduced pressure. The residue was purified by silica gel column chromatography, to afford tert-butyl (2S)-2-amino-3-(6-fluoro-2,3-dimethylphenyl) butanoate (12 g, 68.6%).

Example 1 and 2: 5-((1S,2R)-1-(5-chloro-1,1-dioxido-3-oxobenzo[d]isothiazol-2(3H)-yl)-2-(6-fluoro-2,3-dimethylphenyl)propyl)-1,3,4-oxadiazol-2(3H)-one and 5-((1S,2S)-1-(5-chloro-1,1-dioxido-3-oxobenzo[d]isothiazol-2(3H)-yl)-2-(6-fluoro-2,3-dimethylphenyl)propyl)-1,3,4-oxadiazol-2(3H)-one

### Step 1. methyl 2-(benzylsulfanyl)-5-chlorobenzoate

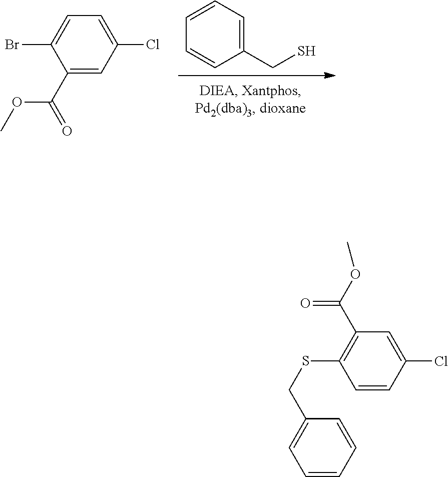

Into a 100 mL round-bottom flask were added methyl 2-bromo-5-chlorobenzoate (1 g, 4.01 mmol, 1.00equiv) in dioxane (10 mL) at room temperature. To the above mixture was added Pd₂(dba)₃ (367.0 mg, 0.40 mmol, 0.1equiv), XantPhos (463 mg, 0.80 mmol, 0.2equiv), DIEA (1.99 mL, 12.0 mmol, 3equiv) and benzyl mercaptan (564.6 uL, 4.81 mmol, 1.2equiv). The resulting mixture was stirred for overnight at 100 degrees C. under nitrogen atmosphere. The mixture was allowed to cool down to room temperature. The resulting mixture was extracted with EtOAc (3×20 mL). The combined organic layers were washed with brine (1×100 mL), dried over anhydrous Na₂SO₄. After filtration, the filtrate was concentrated under reduced pressure. The residue was purified by silica gel column chromatography to afford methyl 2-(benzylsulfanyl)-5-chlorobenzoate (1.08 g, 92.03%).

### Step 2. methyl 5-chloro-2-(chlorosulfonyl) benzoate

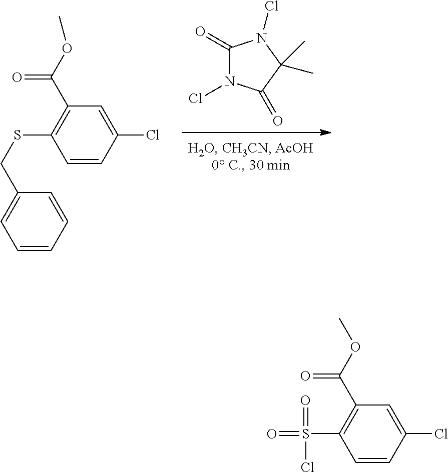 [SMILES: COC(=O)c1cc(Cl)ccc1SCc1ccccc1 ; CC1(C)C(=O)N(Cl)C(=O)N1Cl ; COC(=O)c1cc(Cl)ccc1S(=O)(=O)Cl]

Into a 50 mL 3-necked round-bottom flask were added methyl 2-(benzylsulfanyl)-5-chlorobenzoate (500 mg, 1.708 mmol, 1.00equiv), CH₃CN (0.3 ml). This was followed by the addition of H₂O (0.3 ml), AcOH (5 mL, 87.258 mmol, 51.09equiv), 1,3-dichloro-5,5-dimethylimidazolidine-2,4-dione (672.93 mg, 3.416 mmol, 2equiv) dropwise at 0 degrees C. The resulting mixture was stirred for 30 min at 0 degrees C. under nitrogen atmosphere. The reaction was quenched with Water. The resulting mixture was extracted with EtOAc. The combined organic layers were washed with brine, dried over anhydrous Na₂SO₄. After filtration, the filtrate was concentrated. This resulted in methyl 5-chloro-2-(chlorosulfonyl) benzoate (400 mg, 87%).

### Step 3. methyl 5-chloro-2-(N-((1S)-2-(6-fluoro-2,3-dimethylphenyl)-1-(5-oxo-4,5-dihydro-1,3,4-oxadiazol-2-yl)propyl)sulfamoyl)benzoate

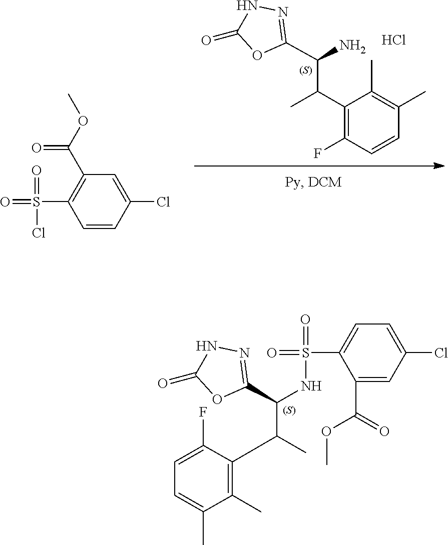

Into a 50 mL round-bottom flask were added 5-[(1S)-1-amino-2-(6-fluoro-2,3-dimethylphenyl)propyl]-3H-1,3,4-oxadiazol-2-one (168.21 mg, 0.557 mmol, 1equiv) in Py (3 mL). To the mixture was added methyl 5-chloro-2-(chlorosulfonyl)benzoate (150 mg, 0.557 mmol, 1.00equiv) in DCM dropwise at 0 degrees C. The resulting mixture was stirred overnight at room temperature. The reaction was quenched with water. The resulting mixture was extracted with CH₂Cl₂. The combined organic layers were washed with brine, dried over anhydrous MgSO₄. After filtration, the filtrate was concentrated. The residue was purified by silica gel column chromatography to afford methyl 5-chloro-2-(N-((1S)-2-(6-fluoro-2,3-dimethylphenyl)-1-(5-oxo-4,5-dihydro-1,3,4-oxadiazol-2-yl)propyl)sulfamoyl)benzoate (150 mg, 54%).

### Step 4. 5-chloro-2-(N-((1S)-2-(6-fluoro-2,3-dimethylphenyl)-1-(5-oxo-4,5-dihydro-1,3,4-oxadiazol-2-yl)propyl)sulfamoyl)benzoic acid

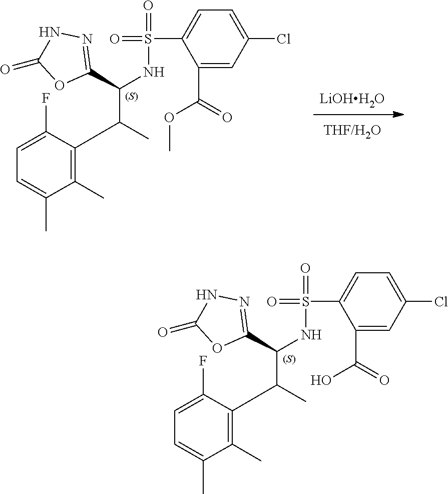

Into a 8 mL vial were added 5-chloro-2-(N-((1S)-2-(6-fluoro-2,3-dimethylphenyl)-1-(5-oxo-4,5-dihydro-1,3,4-oxadiazol-2-yl) propyl)sulfamoyl)benzoic acid (50 mg, 0.100 mmol, 1equiv), THF (3 mL) and LiOH·H₂O (16.85 mg, 0.400 mmol, 4equiv) at room temperature. The resulting mixture was stirred for 2 h at room temperature. The mixture was acidified to pH 7 with HCl (aq.). The resulting mixture was concentrated under vacuum. This resulted in 5-chloro-2-(N-((1S)-2-(6-fluoro-2,3-dimethylphenyl)-1-(5-oxo-4,5-dihydro-1,3,4-oxadiazol-2-yl)propyl)sulfamoyl)benzoic acid (40 mg, 82%).

### Step 5. 5-((1S,2R)-1-(5-chloro-1,1-dioxido-3-oxobenzo[d]isothiazol-2(3H)-yl)-2-(6-fluoro-2,3-dimethylphenyl)propyl)-1,3,4-oxadiazol-2(3H)-one

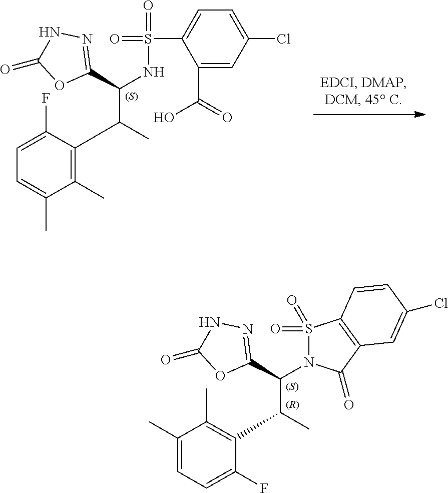

Into a 20 mL vial were added. 5-((1S,2R)-1-(5-chloro-1,1-dioxido-3-oxobenzo[d]isothiazol-2(3H)-yl)-2-(6-fluoro-2,3-dimethylphenyl) propyl)-1,3,4-oxadiazol-2(3H)-one (300 mg, 0.620 mmol, 1.00equiv), DCM (6 mL), EDCI (118.85 mg, 0.620 mmol, 1equiv) and DMAP (227.22 mg, 1.860 mmol, 3equiv). The resulting mixture was stirred for 2 h at 45 degrees C. The residue was purified by reverse flash chromatography. The crude product was purified by Chiral-Prep-HPLC.

First isomer: (20.4 mg, 7%). LC-MS: (ES, m/z): [M+H]: 466.00. ¹H NMR (300 MHz, DMSO-d₆) δ 12.48-12.35 (m, 1H), 8.46 (d, J=8.3 Hz, 1H), 8.33-8.14 (m, 2H), 7.11 (dd, J=8.4, 5.9 Hz, 1H), 6.94 (dd, J=12.2, 8.4 Hz, 1H), 5.79 (d, J=11.6 Hz, 1H), 4.48 (dd, J=12.0, 7.0 Hz, 1H), 2.32 (s, 3H), 2.23 (s, 3H), 1.31 (d, J=6.9 Hz, 3H).

Second isomer: (7.9 mg, 2.7%). LC-MS (ES, m/z): [M+H]: 466.00. ¹H NMR (300 MHz, DMSO-d6) δ 12.62 (s, 1H), 8.28 (d, J=8.3 Hz, 1H), 8.18-8.05 (m, 2H), 7.00 (dd, J=8.4, 5.8 Hz, 1H), 6.83 (dd, J=12.0, 8.3 Hz, 1H), 5.72 (d, J=11.4 Hz, 1H), 4.49 (s, 1H), 2.18 (d, J=26.8 Hz, 6H), 1.43 (d, J=6.9 Hz, 3H).

Example 3 and 4: 5-((1S,2R)-1-(5-chloro-7-methoxy-1,1-dioxido-3-oxobenzo[d]isothiazol-2(3H)-yl)-2-(6-fluoro-2,3-dimethylphenyl)propyl)-1,3,4-oxadiazol-2(3H)-one and 5-((1S,2S)-1-(5-chloro-7-methoxy-1,1-dioxido-3-oxobenzo[d]isothiazol-2(3H)-yl)-2-(6-fluoro-2,3-dimethylphenyl)propyl)-1,3,4-oxadiazol-2(3H)-one

### Step 1. methyl 2-amino-5-chloro-3-methoxybenzoate

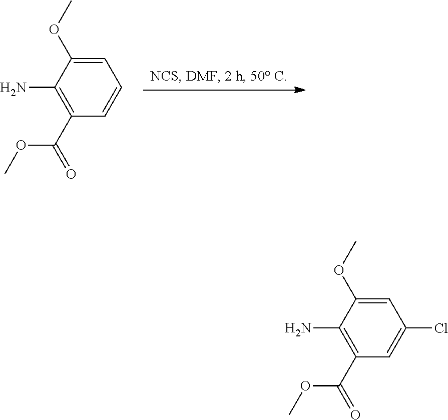

Into a 100 mL round-bottom flask were added methyl 2-amino-3-methoxybenzoate (2 g, 11.038 mmol, 1.00equiv) in DMF (20 mL) and N-chloro succinimide (1.62 g, 12.142 mmol, 1.1equiv) at room temperature. The resulting mixture was stirred for 2 h at 50 degrees C. The resulting mixture was extracted with EtOAc (3×15 mL). The combined organic layers were washed with brine (2×10 mL), dried over anhydrous Na₂SO₄. After filtration, the filtrate was concentrated under reduced pressure. The residue was purified by silica gel column chromatography to afford methyl 2-amino-5-chloro-3-methoxybenzoate (2 g, 84%).

### Step 2. methyl 2-bromo-5-chloro-3-methoxybenzoate

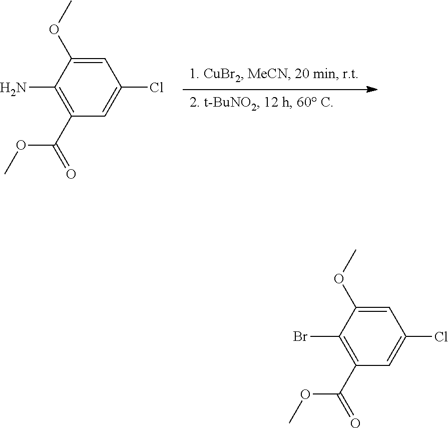

Into a 100 mL round-bottom flask were added methyl 2-amino-5-chloro-3-methoxybenzoate (1 g, 4.638 mmol, 1.00equiv), CuBr₂ (2.07 g, 9.276 mmol, 2.0equiv) and CH₃CN (10.00 mL) at room temperature. The resulting mixture was stirred for 20 min at room temperature. Then tert-butyl nitrite (0.86 g, 8.348 mmol, 1.8equiv) was added. The resulting mixture was stirred overnight at 60 degrees C. The reaction was quenched by the addition of water (10 mL) at room temperature. The resulting mixture was extracted with EtOAc (3×10 mL). The combined organic layers were washed with brine (2×10 mL), dried over anhydrous Na₂SO₄. The residue was purified by silica gel column chromatography to afford methyl 2-bromo-5-chloro-3-methoxybenzoate (0.8 g, 61.72%).

### Step 3. methyl 2-(benzylsulfanyl)-5-chloro-3-methoxybenzoate

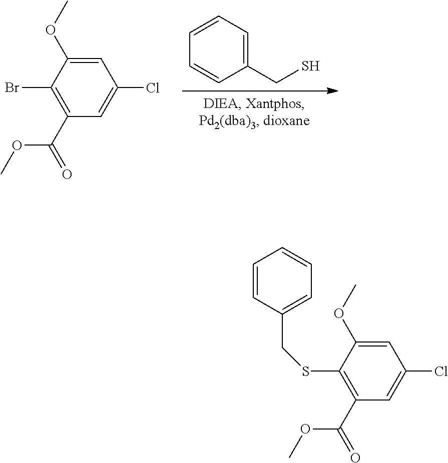

Into a 100 mL round-bottom flask, was placed methyl 2-bromo-5-chloro-3-methoxybenzoate (1.6 g, 5.724 mmol, 1.00equiv), benzyl mercaptan (0.85 g, 6.869 mmol, 1.2equiv), DIEA (2.22 g, 17.172 mmol, 3.0 equiv), Xantphos (0.66 g, 1.145 mmol, 0.2equiv), Pd₂(dba)₃ (0.52 g, 0.572 mmol, 0.1 equiv), dioxane (15 mL). The resulting mixture was stirred overnight at 100 degrees C. under nitrogen atmosphere. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography to afford methyl 2-(benzylsulfanyl)-5-chloro-3-methoxybenzoate (1.2 g, 64%).

### Step 4. methyl 5-chloro-2-(chlorosulfonyl)-3-methoxybenzoate

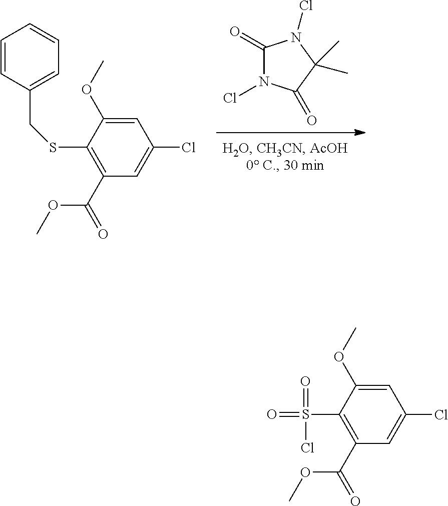 [SMILES: COC(=O)c1cc(Cl)cc(OC)c1SCc1ccccc1 ; CC1(C)C(=O)N(Cl)C(=O)N1Cl ; COC(=O)c1cc(Cl)cc(OC)c1S(=O)(=O)Cl]

Into a 250 mL round-bottom flask, the mixture of methyl 2-(benzylsulfanyl)-5-chloro-3-methoxybenzoate (1.3 g, 4.027 mmol, 1.00 equiv) in MeCN were added 1,3-dichloro-5,5-dimethylimidazolidine-2,4-dione (1.59 g, 8.054 mmol, 2.0 equiv), AcOH (0.90 mL, 15.705 mmol, 3.90equiv) and H₂O (0.70 mL, 38.861 mmol, 9.65equiv) in portions at 0 degrees C. The resulting mixture was stirred for 30 min at 0 degrees C. The resulting mixture was extracted with EtOAc (3×15 mL). The combined organic layers were washed with brine (2×10 mL), dried over anhydrous Na₂SO₄. After filtration, the filtrate was concentrated under reduced pressure to afford methyl 5-chloro-2-(chlorosulfonyl)-3-methoxybenzoate (1 g, 83.01%).

### Step 5. methyl 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoate

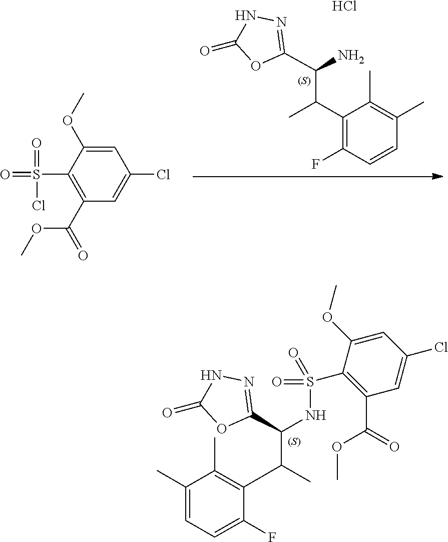

Into a 100 mL round-bottom flask, the mixture of 5-[(1S,2R)-1-amino-2-(6-fluoro-2,3-dimethylphenyl) propyl]-3H-1,3,4-oxadiazol-2-one (200 mg, 0.664 mmol, 1.00 equiv) in C₅H₅N (5 mL) was added methyl 5-chloro-2-(chlorosulfonyl)-3-methoxybenzoate (397.50 mg, 1.328 mmol, 2.0 equiv) dropwise at 0 degrees C. The resulting mixture was stirred overnight at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography to afford methyl 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoate (100 mg, 28%).

### Step 6. 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoic acid

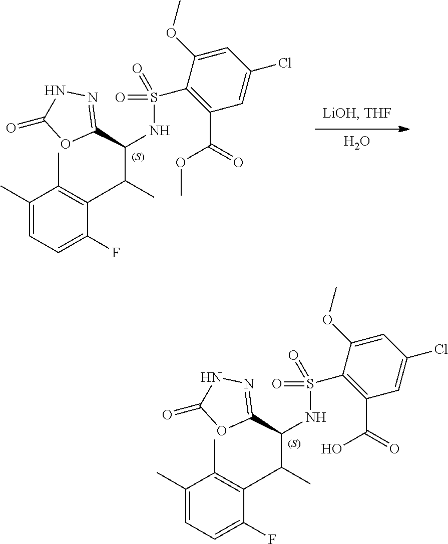

Into a 8 mL round-bottom flask were added 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoate (100 mg, 0.189 mmol, 1.00equiv) in THF (3 mL), water (1 mL) and LiOH·H₂O (15.90 mg, 0.378 mmol, 2.0 equiv) at room temperature. The resulting mixture was stirred for 2 h at 60 degrees C. The mixture was acidified to pH 5. The residue was purified by reverse flash chromatography to afford 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoic acid (70 mg, 71.91%).

Step 7. 5-((1S,2S)-1-(5-chloro-7-methoxy-1,1-dioxido-3-oxobenzo[d]isothiazol-2(3H)-yl)-2-(6-fluoro-2,3-dimethylphenyl)propyl)-1,3,4-oxadiazol-2(3H)-one and 5-((1S,2R)-1-(5-chloro-7-methoxy-1,1-dioxido-3-oxobenzo[d]isothiazol-2(3H)-yl)-2-(6-fluoro-2,3-dimethylphenyl)propyl)-1,3,4-oxadiazol-2(3H)-one

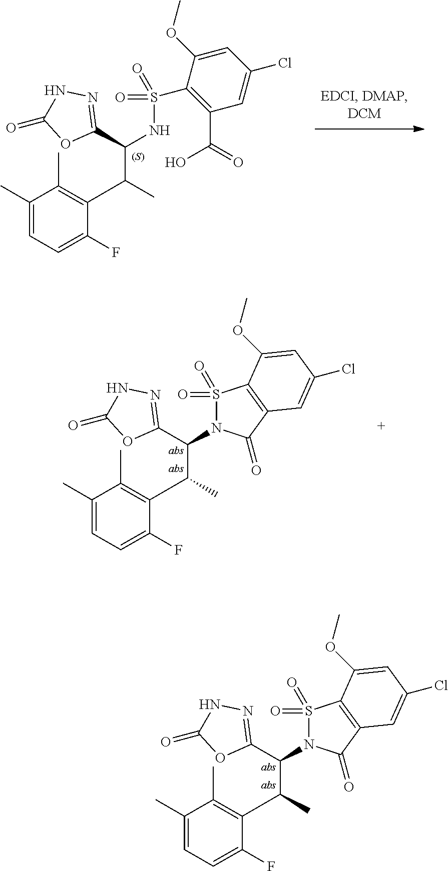

Into a 20 mL round-bottom flask were added 5-chloro-2-{[(1S)-2-(6-fluoro-2,3-dimethylphenyl)-1-(5-oxo-4H-1,3,4-oxadiazol-2-yl)propyl]sulfamoyl}-3-methoxybenzoic acid (120 mg, 0.233 mmol, 1.00equiv), EDCI (89.52 mg, 0.466 mmol, 2.0 equiv), DMAP (2.85 mg, 0.023 mmol, 0.1 equiv) and DCM (3 mL) at room temperature. The resulting mixture was stirred for 2 h at room temperature. The resulting mixture was extracted with CH₂Cl₂ (3×10 mL). The combined organic layers were washed with brine (2×10 mL), dried over anhydrous MgSO₄. After filtration, the filtrate was concentrated under reduced pressure. The residue was purified by reverse flash chromatography. The crude product was purified by Chiral-Prep-HPLC to afford:

First Isomer (9.4 mg, 8%). LC-MS− (ES, m/z): [M+H]: 496.10. 1H NMR (300 MHz, Methanol-d₄) δ 7.79-7.67 (m, 2H), 7.15-7.04 (m, 1H), 6.83 (dd, J=12.1, 8.4 Hz, 1H), 6.01-5.89 (m, 1H), 4.61 (dd, J=12.1, 6.9 Hz, 1H), 4.12 (s, 3H), 2.34 (d, J=36.4 Hz, 6H), 1.38 (d, J=7.0 Hz, 3H).

Second isomer: (1.1 mg, 5.7%). LC-MS: (ES, m/z): [M+H]: 496.10. ¹H NMR (300 MHz, Methanol-d₄) δ 7.73 (d, J=4.7 Hz, 2H), 7.13-7.02 (m, 1H), 6.83 (dd, J=12.0, 8.5 Hz, 1H), 5.95 (d, J=11.7 Hz, 1H), 4.61 (s, 1H), 4.12 (s, 3H), 2.40 (s, 3H), 2.28 (s, 3H), 1.37 (d, J=6.9 Hz, 3H).

Example 5 and 6: 5-((1S,2S)-1-(5-chloro-7-methoxy-1,1-dioxidobenzo[d]isothiazol-2(3H)-yl)-2-(6-fluoro-2,3-dimethylphenyl)propyl)-1,3,4-oxadiazol-2(3H)-one and 5-((1S,2R)-1-(5-chloro-7-methoxy-1,1-dioxidobenzo[d]isothiazol-2(3H)-yl)-2-(6-fluoro-2,3-dimethylphenyl)propyl)-1,3,4-oxadiazol-2(3H)-one

### Step 1. 4-chloro-N-((1S)-2-(6-fluoro-2,3-dimethylphenyl)-1-(5-oxo-4,5-dihydro-1,3,4-oxadiazol-2-yl)propyl)-2-(hydroxymethyl)-6-methoxybenzenesulfonamide

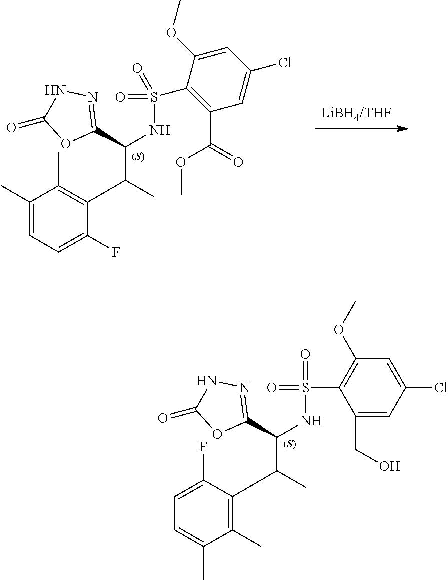

Into a 50 mL round-bottom flask were added 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoate (200 mg, 0.379 mmol, 1.00equiv), THF (3 mL) and LiBH₄ 2M in THF (284.12 uL, 0.569 mmol, 1.5equiv) at 0 degrees C. The resulting mixture was stirred for 2 h at room temperature. The reaction was quenched with water. The residue was purified by reverse flash chromatography. This resulted in 4-chloro-N-[(1S)-2-(6-fluoro-2,3-dimethylphenyl)-1-(5-oxo-4H-1,3,4-oxadiazol-2-yl) propyl]-2-(hydroxymethyl)-6-methoxybenzenesulfonamide (50 mg, 26.40%).

### Step 2. 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzyl methanesulfonate

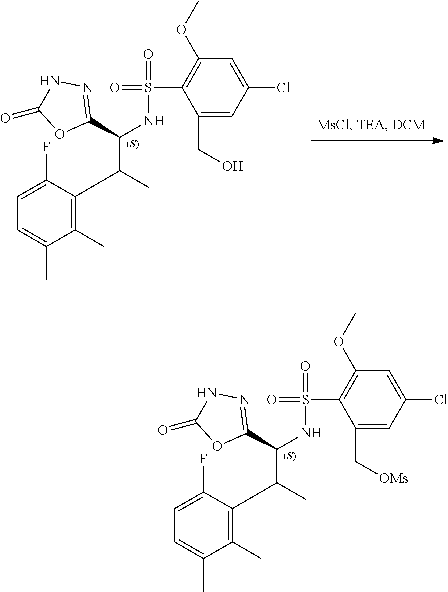

Into an 8 mL vial were added 4-chloro-N-[(1S,2R)-2-(6-fluoro-2,3-dimethylphenyl)-1-(5-oxo-4H-1,3,4-oxadiazol-2-yl) propyl]-2-(hydroxymethyl)-6-methoxybenzenesulfonamide (100 mg, 0.200 mmol, 1.00equiv), DCM (3 mL) and TEA (83.41 uL, 0.600 mmol, 3equiv). This was followed by the addition of MsCl (600.07 uL, 0.600 mmol, 3equiv) dropwise at 0 degrees C. The resulting mixture was stirred overnight at room temperature. The residue was purified by Prep-TLC to afford 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzyl methanesulfonate (50 mg, 43.25%).

Step 3. 5-((1S,2S)-1-(5-chloro-7-methoxy-1,1-dioxidobenzo[d]isothiazol-2(3H)-yl)-2-(6-fluoro-2,3-dimethylphenyl) propyl)-1,3,4-oxadiazol-2(3H)-one and 5-((1S,2R)-1-(5-chloro-7-methoxy-1,1-dioxidobenzo[d]isothiazol-2(3H)-yl)-2-(6-fluoro-2,3-dimethylphenyl) propyl)-1,3,4-oxadiazol-2(3H)-one

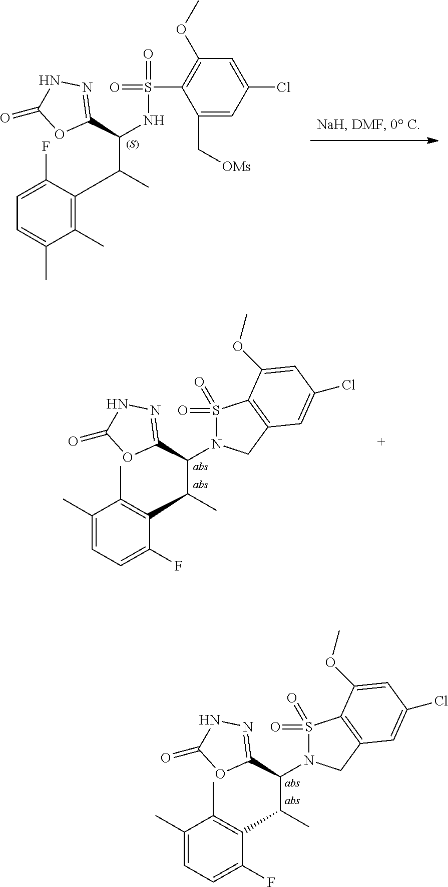

Into a 25 mL round-bottom flask were 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzyl methanesulfonate (50 mg, 0.087 mmol, 1.00equiv) and DMF (2 mL). This was followed by the addition of NaH (11 mg, 0.46 mmol, 5.30equiv) dropwise at 0 degrees C. The resulting mixture was stirred for 1 h at room temperature under nitrogen atmosphere. The reaction was quenched with water. The resulting mixture was extracted with EtOAc. The combined organic layers were washed with brine, dried over anhydrous Na₂SO₄. After filtration, the filtrate was concentrated. The residue was purified by Prep-TLC. The crude product was purified by Chiral-Prep-HPLC. This resulted in:

First isomer: (2.7 mg, 6.5%). LC-MS: (ES, m/z): [M−H]: 480.05. ¹H NMR (300 MHz, Methanol-d₄) δ 7.15-6.94 (m, 3H), 6.81 (dd, J=12.0, 8.4 Hz, 1H), 5.37 (d, J=11.4 Hz, 1H), 4.70 (d, J=14.7 Hz, 1H), 4.13 (d, J=14.6 Hz, 1H), 3.92 (d, J=9.3 Hz, 4H), 3.79 (s, 1H), 2.40 (s, 3H), 2.26 (s, 3H), 1.35 (d, J=6.9 Hz, 3H).

Second isomer: (9.1 mg, 21.8%). LC-MS: (ES, m/z): [M+H]: 482.10. ¹H NMR (300 MHz, Methanol-d₄) δ 7.24 (d, J=3.9 Hz, 2H), 7.06 (dd, J=8.4, 5.8 Hz, 1H), 6.83 (dd, J=12.1, 8.4 Hz, 1H), 5.29 (d, J=11.6 Hz, 1H), 4.83-4.69 (m, 2H), 4.03 (s, 4H), 2.34 (s, 3H), 2.25 (s, 3H), 1.44 (dd, J=7.0, 1.2 Hz, 3H).

Example 7 and 8: Synthesis of 5-((1S,2S)-1-(6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one and 5-((1S,2R)-1-(6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

### Step 1. methyl 2-[(diphenylmethylidene)amino]acetate

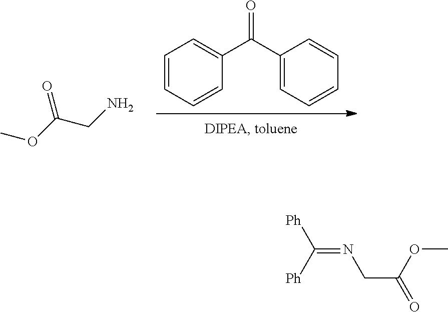

To a stirred mixture of methyl 2-aminoacetate hydrochloride (12.6 g, 100.358 mmol, 1 equiv) in toluene was added benzophenone (36.58 g, 200.716 mmol, 2 equiv) at room temperature. The resulting mixture was stirred for 30 min at 120° C. To the above mixture was added DIPEA (25.94 g, 200.716 mmol, 2 equiv) dropwise over 3 h at 120° C. The resulting mixture was stirred for additional 3 h at 120° C. The resulting mixture was concentrated under vacuum. The residue was purified by silica gel column chromatography to afford methyl 2-[(diphenylmethylidene)amino]acetate (8 g, 31.47%).

### Step 2. methyl 2-((diphenylmethylene)amino)-3-(6-fluoro-2,3-dimethylphenyl) butanoate

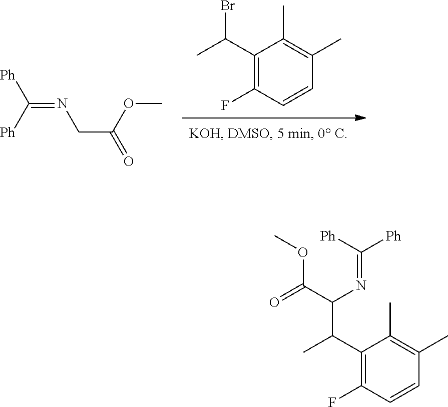 [SMILES: COC(=O)CN=C(c1ccccc1)c1ccccc1 ; Cc1ccc(F)c(C(C)Br)c1C ; COC(=O)C(N=C(c1ccccc1)c1ccccc1)C(C)c1c(F)ccc(C)c1C]

To a stirred mixture of methyl 2-[(diphenylmethylidene)amino]acetate (8 g, 31.583 mmol, 1 equiv) in DMSO was added KOH (5.32 g, 94.821 mmol, 3.00 equiv) in portions at 25° C. The resulting mixture was stirred for 5 min at 25° C. To the above mixture was added 2-(1-bromoethyl)-1-fluoro-3,4-dimethylbenzene (7.30 g, 31.587 mmol, 1.00 equiv) dropwise at 25° C. The resulting mixture was stirred for additional 2 h at room temperature. The mixture was acidified to pH 7 with HCl (2M). The resulting mixture was extracted with EtOAc (3×100 mL). The combined organic layers were washed with brine (2×200 mL), dried over anhydrous Na₂SO₄. After filtration, the filtrate was concentrated under reduced pressure. The crude product was used in the next step directly without further purification.

### Step 3. methyl 2-amino-3-(6-fluoro-2,3-dimethylphenyl) butanoate, formic acid

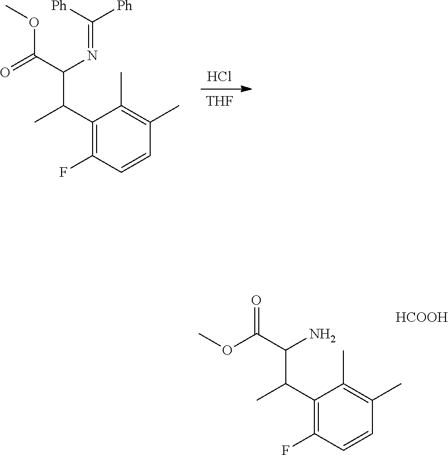

To a stirred mixture of methyl 2-[(diphenylmethylidene)amino]-3-(6-fluoro-2,3-dimethylphenyl) butanoate (8 g, 19.827 mmol, 1 equiv) in THF was added hydrogen chloride (5 mL, 137.137 mmol, 6.92 equiv) dropwise at room temperature. The resulting mixture was stirred for 10 min at room temperature. The mixture basified to pH 7 with NaOH (1N). The resulting mixture was extracted with EtOAc (3×25 mL). The combined organic layers were washed with brine (1×50 mL), dried over anhydrous Na₂SO₄. After filtration, the filtrate was concentrated under reduced pressure. The residue was purified by reverse flash chromatography. This resulted in formic acid; methyl 2-amino-3-(6-fluoro-2,3-dimethylphenyl) butanoate (2.5 g, 44.19%).

### Step 4. tert-butyl 2-(2-bromo-5-chlorophenyl) acetate

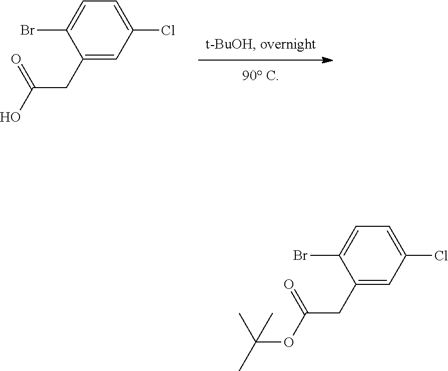 [SMILES: O=C(O)Cc1cc(Cl)ccc1Br ; CC(C)(C)OC(=O)Cc1cc(Cl)ccc1Br]

Into a 250 mL round-bottom flask were added (2-bromo-5-chlorophenyl) acetic acid (5 g, 20.041 mmol, 1.00 equiv) and t-BuOH (50 mL) at room temperature. To the mixture was added (Boc)₂O (25 g, 114.6 mmol, 5.72 equiv). The resulting mixture was stirred for overnight at 90° C. The reaction was quenched with water. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by silica gel column chromatography to afford tert-butyl 2-(2-bromo-5-chlorophenyl) acetate (5 g, 81.64%).

### Step 5. tert-butyl 2-[2-(benzylsulfanyl)-5-chlorophenyl]acetate

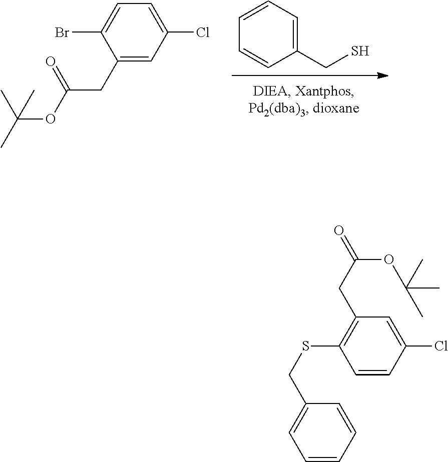

Into a 40 mL vial were added tert-butyl 2-(2-bromo-5-chlorophenyl)acetate (2 g, 6.545 mmol, 1.00 equiv) and dioxane (6 mL, 82.636 mmol), DIEA (2.54 g, 19.635 mmol, 3 equiv), benzyl mercaptan (0.98 g, 7.854 mmol, 1.2 equiv), XantPhos (378.68 mg, 0.655 mmol, 0.1 equiv), Pd₂(dba)₃ (299.65 mg, 0.327 mmol, 0.05 equiv). The resulting mixture was stirred overnight at 100° C. under nitrogen atmosphere. The reaction was quenched with water. The resulting mixture was extracted with EtOAc (3×25 mL). The combined organic layers were washed with brine (1×50 mL), dried over anhydrous Na₂SO₄. After filtration, the filtrate was concentrated under reduced pressure. The residue was purified by silica gel column chromatography to afford tert-butyl 2-[2-(benzylsulfanyl)-5-chlorophenyl]acetate (1.5 g, 65.7%).

### Step 6. tert-butyl 2-[5-chloro-2-(chlorosulfonyl)phenyl]acetate

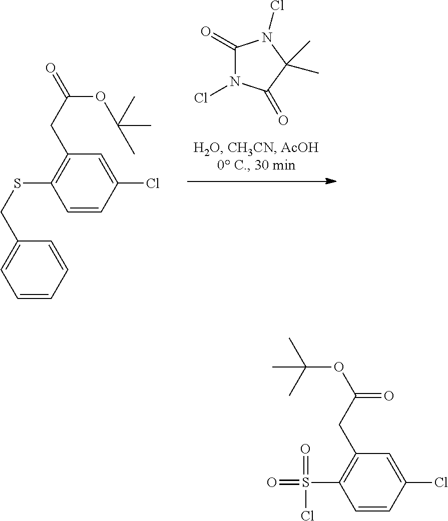 [SMILES: CC(C)(C)OC(=O)Cc1cc(Cl)ccc1SCc1ccccc1 ; CC1(C)C(=O)N(Cl)C(=O)N1Cl ; CC(C)(C)OC(=O)Cc1cc(Cl)ccc1S(=O)(=O)Cl]

Into a 50 mL round-bottom flask were added tert-butyl 2-[2-(benzylsulfanyl)-5-chlorophenyl]acetate (5.5 g, 15.850 mmol, 1.00 equiv), MECN (30 mL, 76.099 mmol), CH₃COOH (3.6 mL, 21.093 mmol), H₂O (1.8 mL, 21.093 mmol). To the above mixture was added 1,3-dichloro-5,5-dimethylimidazolidine-2,4-dione (6.25 g, 31.700 mmol, 2 equiv) at 0° C. The resulting mixture was stirred for additional 30 min at ° C. The reaction was quenched with water. The resulting mixture was extracted with EtOAc (3×20 mL). The combined organic layers were washed with brine (1×100 mL), dried over anhydrous Na₂SO₄. After filtration, the filtrate was concentrated under reduced pressure. The residue was purified by silica gel column chromatography to afford tert-butyl 2-[5-chloro-2-(chlorosulfonyl) phenyl]acetate (3.65 g, 70.8%).

### Step 7. methyl 2-((2-(2-(tert-butoxy)-2-oxoethyl)-4-chlorophenyl) sulfonamido)-3-(6-fluoro-2,3-dimethylphenyl)butanoate

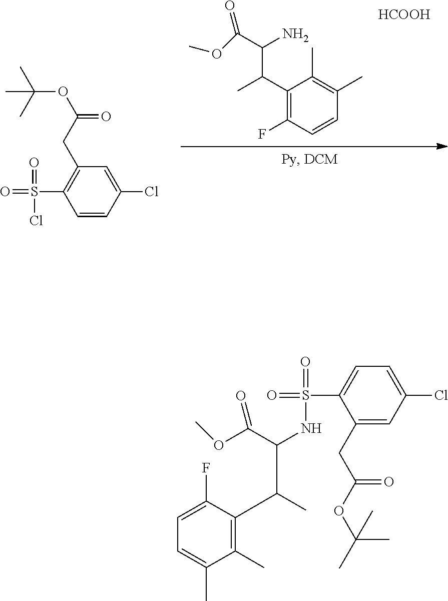

To a stirred mixture of methyl 2-amino-3-(6-fluoro-2,3-dimethylphenyl) butanoate (2 g, 8.358 mmol, 1 equiv) in pyridine was added tert-butyl 2-[5-chloro-2-(chlorosulfonyl)phenyl]acetate (3.67 g, 11.283 mmol, 1.35 equiv) dropwise at 0° C. The resulting mixture was stirred for 60 min at room temperature. The resulting mixture was concentrated under vacuum. The residue was purified by reverse flash chromatography. This resulted in methyl 2-((2-(2-(tert-butoxy)-2-oxoethyl)-4-chlorophenyl) sulfonamido)-3-(6-fluoro-2,3-dimethylphenyl)butanoate (4.2 g, 95.1%).

### Step 8. 2-(5-chloro-2-(N-(3-(6-fluoro-2,3-dimethylphenyl)-1-methoxy-1-oxobutan-2-yl) sulfamoyl) phenyl) acetic acid

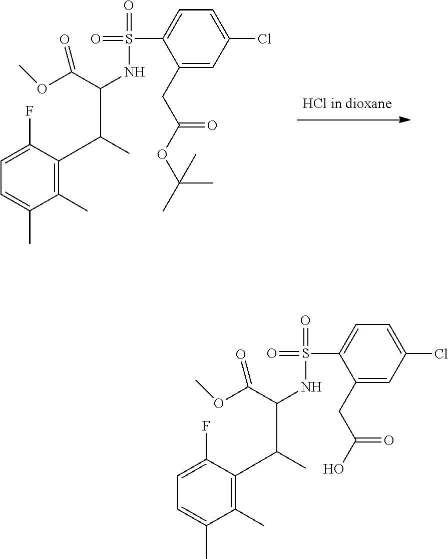 [SMILES: COC(=O)C(NS(=O)(=O)c1ccc(Cl)cc1CC(=O)OC(C)(C)C)C(C)c1c(F)ccc(C)c1C ; COC(=O)C(NS(=O)(=O)c1ccc(Cl)cc1CC(=O)O)C(C)c1c(F)ccc(C)c1C]

To a stirred mixture of methyl 2-((2-(2-(tert-butoxy)-2-oxoethyl)-4-chlorophenyl) sulfonamido)-3-(6-fluoro-2,3-dimethylphenyl)butanoate (4.3 g, 8.143 mmol, 1 equiv) is added HCl 4N in 1,4-dioxane (40 mL, 819.919 mmol, 100.69 equiv) at room temperature. The resulting mixture was stirred for 1 h at 45° C. under air atmosphere. The resulting mixture was concentrated under vacuum. The residue was purified by reverse flash chromatography. This resulted in 2-(5-chloro-2-(N-(3-(6-fluoro-2,3-dimethylphenyl)-1-methoxy-1-oxobutan-2-yl)sulfamoyl)phenyl)acetic acid (2.4 g, 62.5%).

### Step 9. 2-[4-chloro-2-(2-hydroxyethyl) benzenesulfonamido]-3-(6-fluoro-2,3-dimethylphenyl) butanoate

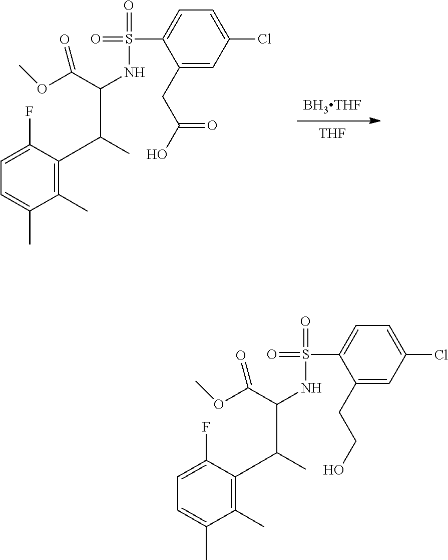 [SMILES: COC(=O)C(NS(=O)(=O)c1ccc(Cl)cc1CC(=O)O)C(C)c1c(F)ccc(C)c1C ; COC(=O)C(NS(=O)(=O)c1ccc(Cl)cc1CCO)C(C)c1c(F)ccc(C)c1C ; "BH3·THF" ; "THF"]

To a stirred mixture of BH₃·THF (2.92 mL, 2.924 mmol, 1.2 equiv) was added 2-(5-chloro-2-(N-(3-(6-fluoro-2,3-dimethylphenyl)-1-methoxy-1-oxobutan-2-yl)sulfamoyl)phenyl)acetic acid (1.15 g, 2.437 mmol, 1 equiv) in THF (10 ml) dropwise at room temperature under nitrogen atmosphere. The resulting mixture was stirred for 10 min at room temperature. The reaction was quenched by the addition of MeOH (0.5 mL) at room temperature. The resulting mixture was concentrated under reduced pressure. The residue was purified by reverse flash chromatography. This resulted in methyl 2-[4-chloro-2-(2-hydroxyethyl) benzenesulfonamido]-3-(6-fluoro-2,3-dimethylphenyl)butanoate (770 mg, 63.5%).

### Step 10. methyl 2-((4-chloro-2-(2-((methylsulfonyl)oxy)ethyl)phenyl)sulfonamido)-3-(6-fluoro-2,3-dimethylphenyl)butanoate

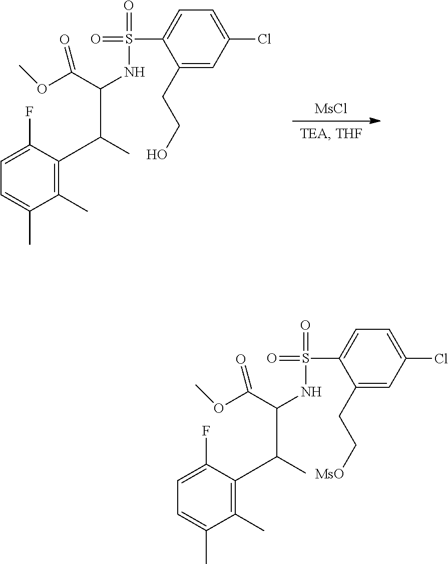 [SMILES: "MsCl" ; COC(=O)C(NS(=O)(=O)c1ccc(Cl)cc1CCO)C(C)c1c(F)ccc(C)c1C ; "TEA, THF" ; COC(=O)C(NS(=O)(=O)c1ccc(Cl)cc1CCOS(C)(=O)=O)C(C)c1c(F)ccc(C)c1C]

Into a 100 mL round-bottom flask were added methyl 2-[4-chloro-2-(2-hydroxyethyl) benzenesulfonamido]-3-(6-fluoro-2,3-dimethylphenyl) butanoate (770 mg, 1.681 mmol, 1 equiv) and DCM (4 mL). To the above mixture was added 2M MsCl in DCM (1.68 mL, 3.844 mmol, 2 equiv) dropwise at 0° C. The resulting mixture was stirred for additional 1 h at room temperature. The reaction was quenched with water. The resulting mixture was extracted with CH₂Cl₂ (3×15 mL). The combined organic layers were washed with brine (1×25 mL), dried over anhydrous Na₂SO₄. The residue was purified by silica gel column chromatography to afford methyl 2-((4-chloro-2-(2-((methyl sulfonyl) oxy) ethyl) phenyl)sulfonamido)-3-(6-fluoro-2,3-dimethylphenyl)butanoate (745 mg, 82.66%).

### Step 11. methyl 2-(6-chloro-1,1-dioxido-3,4-dihydro-2H-benzo[e][1,2]thiazin-2-yl)-3-(6-fluoro-2,3-dimethylphenyl)butanoate

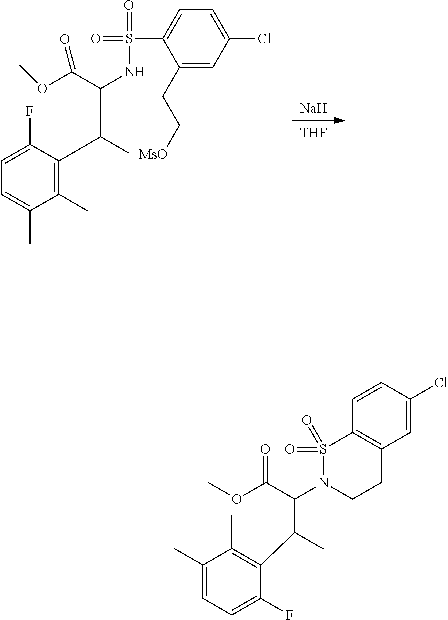

Into a 100 mL 3-necked round-bottom flask were added methyl 2-((4-chloro-2-(2-((methylsulfonyl)oxy) ethyl)phenyl)sulfonamido)-3-(6-fluoro-2,3-dimethylphenyl)butanoate (740 mg, 1.381 mmol, 1 equiv) and tetrahydrofuran (7.73 mL, 107.202 mmol, 77.65 equiv). To the above mixture was added sodium hydride (83 mg, 3.459 mmol, 2.51 equiv) at 0° C. The resulting mixture was stirred overnight at room temperature under nitrogen atmosphere. The reaction was quenched with water. The residue was purified by reverse flash chromatography. This resulted in methyl 2-(6-chloro-1,1-dioxido-3,4-dihydro-2H-benzo[e][1,2]thiazin-2-yl)-3-(6-fluoro-2,3-dimethylphenyl)butanoate (600 mg, 98.79%).

### Step 12. 2-(6-chloro-1,1-dioxido-3,4-dihydro-2H-benzo[e][1,2]thiazin-2-yl)-3-(6-fluoro-2,3-dimethylphenyl)butanoic acid

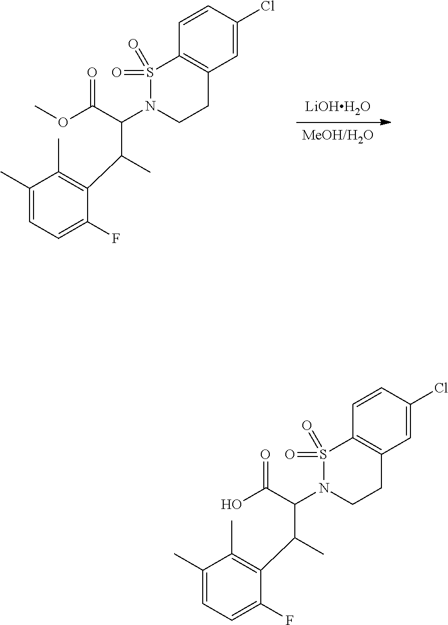 [SMILES: COC(=O)C(C(C)c1c(F)ccc(C)c1C)N1CCc2cc(Cl)ccc2S1(=O)=O ; "LiOH·H2O" ; "MeOH/H2O" ; Cc1ccc(F)c(C(C)C(C(=O)O)N2CCc3cc(Cl)ccc3S2(=O)=O)c1C]

To a stirred mixture of methyl 2-(6-chloro-1,1-dioxido-3,4-dihydro-2H-benzo[e][1,2]thiazin-2-yl)-3-(6-fluoro-2,3-dimethylphenyl)butanoate (600 mg, 1.364 mmol, 1 equiv) and MeOH (5 mL, 123.495 mmol, 90.55 equiv) in water was added lithium hydroxide (181.22 mg, 4.320 mmol, 5 equiv) in portions at room temperature. The resulting mixture was stirred for 4 h at 60° C. The mixture was allowed to cool down to room temperature. The mixture was acidified to pH 6 with HCl (2M). The resulting mixture was concentrated under vacuum. The residue was purified by reverse flash chromatography. This resulted in 2-(6-chloro-1,1-dioxido-3,4-dihydro-2H-benzo[e][1,2]thiazin-2-yl)-3-(6-fluoro-2,3-dimethylphenyl)butanoic acid (404 mg, 69.6%).

### Step 13. tert-butyl 2-(2-(6-chloro-1,1-dioxido-3,4-dihydro-2H-benzo[e][1,2]thiazin-2-yl)-3-(6-fluoro-2,3-dimethylphenyl)butanoyl)hydrazine-1-carboxylate

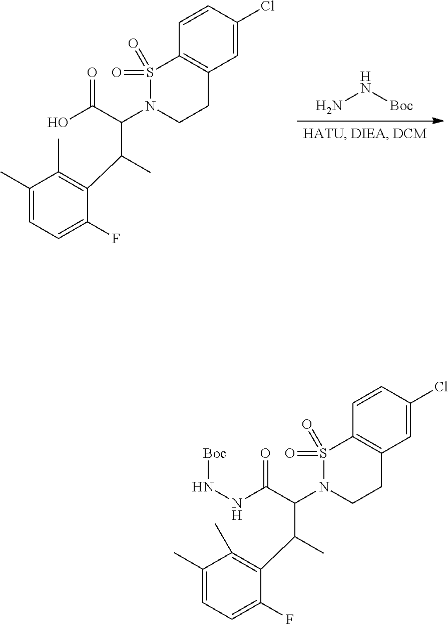

To a stirred mixture of 2-(6-chloro-1,1-dioxido-3,4-dihydro-2H-benzo[e][1,2]thiazin-2-yl)-3-(6-fluoro-2,3-dimethylphenyl)butanoic acid (460 mg, 1.080 mmol, 1 equiv) and HATU (616.01 mg, 1.620 mmol, 1.5 equiv) in DCM was added tert-butoxycarbohydrazide (214.12 mg, 1.620 mmol, 1.5 equiv) in portions at room temperature. To the above mixture was added DIEA (564.38 μL, 3.240 mmol, 3 equiv) dropwise at 0° C. The resulting mixture was stirred for additional 20 min at room temperature. The resulting mixture was concentrated under vacuum. The residue was purified by reverse flash chromatography. This resulted in tert-butyl 2-(2-(6-chloro-1,1-dioxido-3,4-dihydro-2H-benzo[e][1,2]thiazin-2-yl)-3-(6-fluoro-2,3-dimethylphenyl)butanoyl)hydrazine-1-carboxylate (583 mg, 99.9%).

### Step 14. 2-(6-chloro-1,1-dioxido-3,4-dihydro-2H-benzo[e][1,2]thiazin-2-yl)-3-(6-fluoro-2,3-dimethylphenyl)butane hydrazide

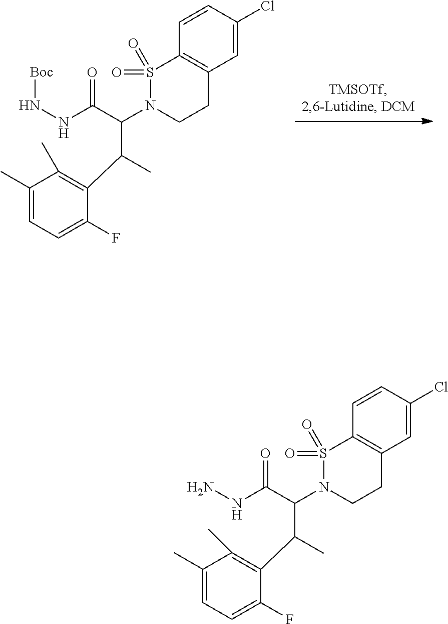 [SMILES: Cc1ccc(F)c(C(C)C(C(=O)NNC(=O)OC(C)(C)C)N2CCc3cc(Cl)ccc3S2(=O)=O)c1C ; "TMSOTf,2,6-Lutidine, DCM" ; Cc1ccc(F)c(C(C)C(C(=O)NN)N2CCc3cc(Cl)ccc3S2(=O)=O)c1C]

Into a 100 mL round-bottom flask were added tert-butyl 2-(2-(6-chloro-1,1-dioxido-3,4-dihydro-2H-benzo[e][1,2]thiazin-2-yl)-3-(6-fluoro-2,3-dimethylphenyl)butanoyl)hydrazine-1-carboxylate (550 mg, 1.018 mmol, 1 equiv) and 2,6-lutidine (2372.33 μL, 20.360 mmol, 20 equiv). To the above mixture was added TMSOTf (3621.65 mg, 16.288 mmol, 16 equiv) at 0° C. The resulting mixture was stirred for additional 1 h at room temperature. The resulting mixture was concentrated under vacuum. The residue was purified by reverse flash chromatography. This resulted in 2-(6-chloro-1,1-dioxido-3,4-dihydro-2H-benzo[e][1,2]thiazin-2-yl)-3-(6-fluoro-2,3-dimethylphenyl)butanehydrazide (430 mg, 95.97%).

### Step 15. 5-(1-(6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

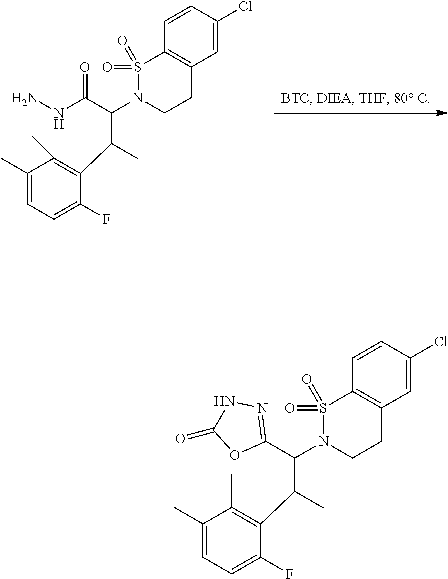 [SMILES: Cc1ccc(F)c(C(C)C(C(=O)NN)N2CCc3cc(Cl)ccc3S2(=O)=O)c1C ; "BTC, DIEA, THF, 80° C." ; Cc1ccc(F)c(C(C)C(c2n[nH]c(=O)o2)N2CCc3cc(Cl)ccc3S2(=O)=O)c1C]

Into a 20 mL vial were added 2-(6-chloro-1,1-dioxido-3,4-dihydro-2H-benzo[e][1,2]thiazin-2-yl)-3-(6-fluoro-2,3-dimethylphenyl)butanehydrazide (430 mg, 0.341 mmol, 1 equiv), DIEA (392.96 μL, 2.255 mmol, 2.5 equiv) and ditrichloromethyl carbonate (133.90 mg, 0.451 mmol, 0.5 equiv) in THF. The resulting mixture was stirred for 1 h at 80° C. The resulting mixture was concentrated under vacuum. The residue was purified by reverse flash chromatography. This resulted in 5-(1-(6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (375 mg, 89.19%).

### Step 16. 5-((1S,2S)-1-(6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

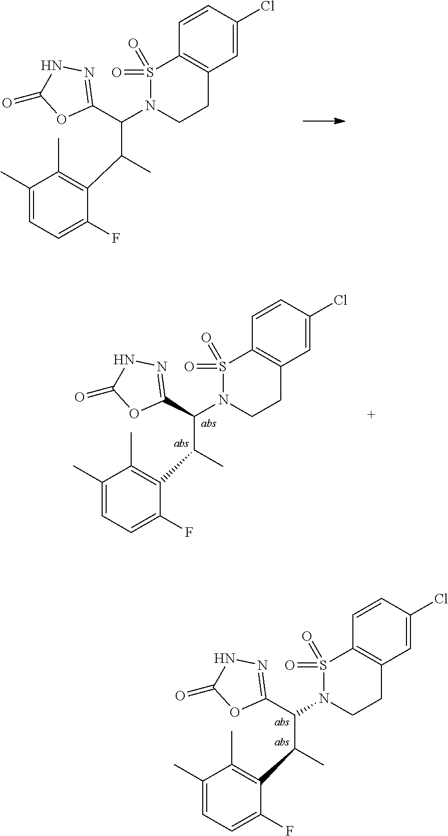

The crude product was purified by Chiral-Prep-HPLC. This resulted in 2 main products:

First isomer: (21.6 mg, 4.29%). LC-MS: (ES, m/z): [M−H]: 464.00. H-NMR (CD₃OD, ppm): 1H NMR (300 MHz, Methanol-d₄) δ 7.72 (d, J=8.4 Hz, 1H), 7.44-7.36 (m, 2H), 7.02-6.97 (dd, J=8.4, 5.8 Hz, 1H), 6.77-6.70 (dd, J=12.1, 8.4 Hz, 1H), 5.53-5.48 (dd, J=11.7, 1.5 Hz, 1H), 4.09-3.80 (m, 3H), 3.21-2.94 (m, 2H), 2.30 (s, 3H), 2.20 (s, 3H), 1.43-1.40 (dd, J=6.9, 1.2 Hz, 3H).

Second isomer: (28.7 mg, 17.94%). LC-MS m/z: 464 (M−1). ¹H NMR (300 MHz, Methanol-d₄) δ 7.56 (d, J=8.4 Hz, 1H), 7.33 (dd, J=8.4, 2.1 Hz, 1H), 7.22 (d, J=2.0 Hz, 1H), 7.00 (dd, J=8.4, 5.7 Hz, 1H), 6.72 (dd, J=12.0, 8.4 Hz, 1H), 5.45 (d, J=11.3 Hz, 1H), 3.91 (dq, J=13.6, 6.9 Hz, 1H), 3.71 (dt, J=14.0, 7.2 Hz, 1H), 3.60-3.45 (m, 1H), 2.92-2.64 (m, 2H), 2.39-2.15 (m, 6H), 1.25 (d, J=6.8 Hz, 3H).

Example 9 and 10: 5-((1S,2S)-1-(7-chloro-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one and 5-((1S,2R)-1-(7-chloro-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one

### Step 1. methyl (2S)-2-(2-bromo-4-chlorobenzenesulfonamido)-3-(6-fluoro-2,3-dimethylphenyl) butanoate

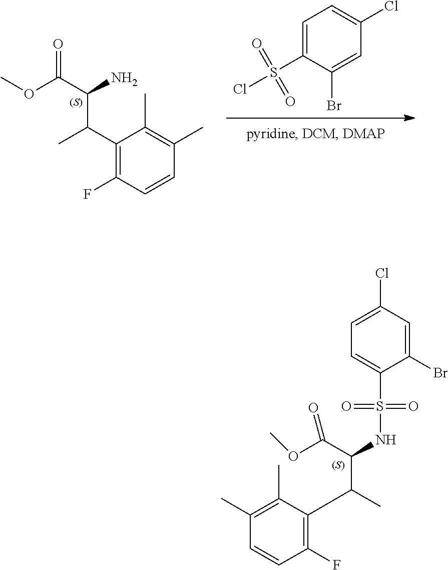

Into a 100 mL round-bottom flask were added methyl (2S)-2-amino-3-(6-fluoro-2,3-dimethylphenyl) butanoate (1 g, 4.179 mmol, 1.00 equiv), DMAP (51.05 mg, 0.418 mmol, 0.1 equiv), pyridine (10 mL) at room temperature. To the above mixture was added 2-bromo-4-chlorobenzenesulfonyl chloride (1.82 g, 6.269 mmol, 1.5 equiv) in DCM (10 mL) dropwise at 0° C. The resulting mixture was stirred for additional 2 h at room temperature. The resulting mixture was extracted with EtOAc (2×100 mL). The combined organic layers were washed with brine (1×100 mL), dried over anhydrous Na₂SO₄. After filtration, the filtrate was concentrated under reduced pressure. The residue was purified by silica gel column chromatography to afford methyl (2S)-2-(2-bromo-4-chlorobenzenesulfonamido)-3-(6-fluoro-2,3-dimethylphenyl) butanoate (1.75 g, 84.98%).

### Step 2. methyl (2S)-2-(4-chloro-2-ethenylbenzenesulfonamido)-3-(6-fluoro-2,3-dimethylphenyl) butanoate

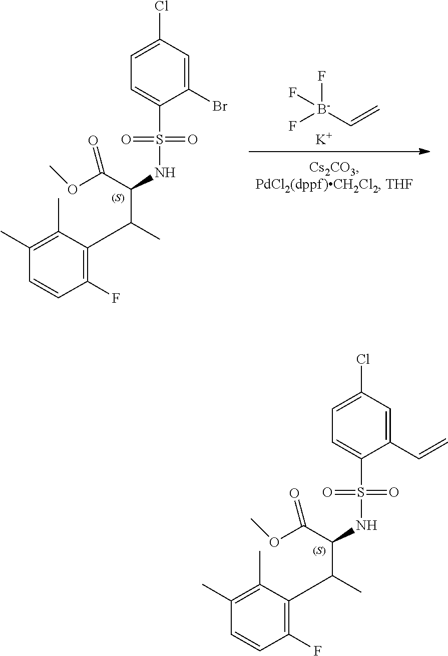

Into a 20 mL sealed tube were added methyl (2S)-2-(2-bromo-4-chlorobenzenesulfonamido)-3-(6-fluoro-2,3-dimethylphenyl) butanoate (1 g, 2.029 mmol, 1 equiv), potassium ethenyltrifluoroboranuide (299.01 mg, 2.232 mmol, 1.1 equiv), Cs₂CO₃ (1.98 g, 6.087 mmol, 3 equiv), THF (5 mL), H₂O (5 mL) and Pd(PPh₃)₂Cl₂ (142.44 mg, 0.203 mmol, 0.1 equiv) at room temperature. The resulting mixture was stirred for 1.5 h at 90° C. under nitrogen atmosphere. The resulting mixture was extracted with EtOAc (2×100 mL). The combined organic layers were washed with brine (1×100 mL), dried over anhydrous Na₂SO₄. After filtration, the filtrate was concentrated under reduced pressure. The residue was purified by silica gel column chromatography to afford methyl (2S)-2-(4-chloro-2-ethenylbenzenesulfonamido)-3-(6-fluoro-2,3-dimethylphenyl) butanoate (780 mg, 87.4%).

### Step 3. (2S)-3-(6-fluoro-2,3-dimethylphenyl)-2-[N-(prop-2-en-1-yl)4-chloro-2-ethenylbenzenesulfonamido]butanoate

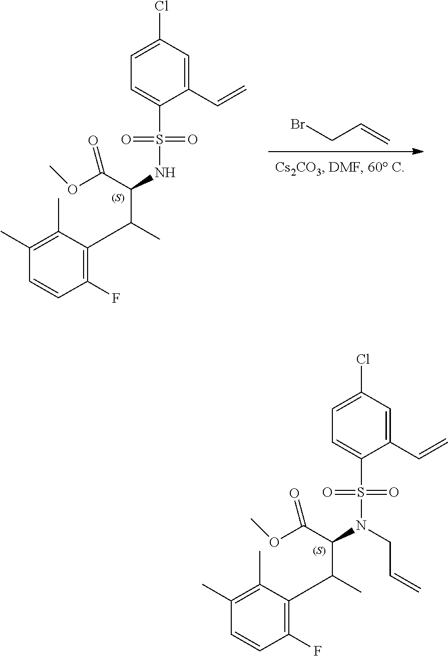

Into a 100 mL round-bottom flask were added methyl (2S)-2-(4-chloro-2-ethenylbenzenesulfonamido)-3-(6-fluoro-2,3-dimethylphenyl) butanoate (1.5 g, 3.410 mmol, 1 equiv), allyl bromide (412.49 mg, 3.410 mmol, 1 equiv), Cs₂CO₃ (3.33 g, 10.230 mmol, 3 equiv) and DMF (15 mL) at room temperature. The resulting mixture was stirred for 1 h at 60° C. under air atmosphere. The resulting mixture was extracted with EtOAc (2×100 mL). The combined organic layers were washed with brine (1×100 mL), dried over anhydrous Na₂SO₄. After filtration, the filtrate was concentrated under reduced pressure. The residue was purified by silica gel column chromatography to afford methyl (2S)-3-(6-fluoro-2,3-dimethylphenyl)-2-[N-(prop-2-en-1-yl)4-chloro-2-ethenylbenzenesulfonamido]butanoate (1.4 g, 85.54%).

### Step 4. methyl (2S)-2-(7-chloro-1,1-dioxidobenzo[f][1,2]thiazepin-2(3H)-yl)-3-(6-fluoro-2,3-dimethylphenyl)butanoate

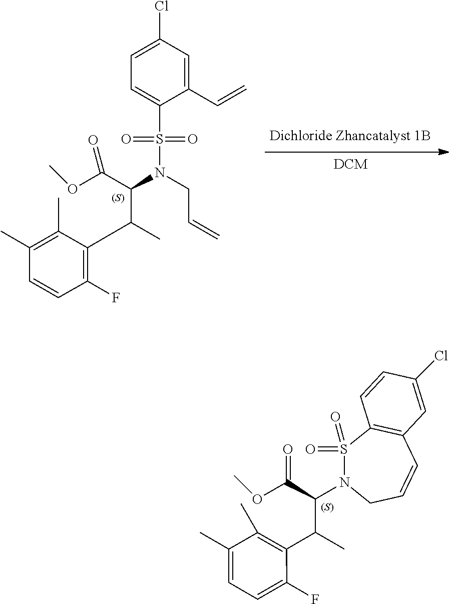

Into a 100 mL round-bottom flask were added methyl (2S)-3-(6-fluoro-2,3-dimethylphenyl)-2-[N-(prop-2-en-1-yl)4-chloro-2-ethenylbenzenesulfonamido]butanoate (1.68 g, 3.500 mmol, 1 equiv), [1,3-bis(2,4,6-trimethylphenyl)imidazolidin-2-ylidene]({[5-(dimethylsulfamoyl)-2-isopropoxyphenyl]methylidene})rutheniumdiuide dichloride (128.41 mg, 0.175 mmol, 0.05 equiv) and DCM (20 mL) at room temperature. The resulting mixture was stirred overnight at room temperature under air atmosphere. The residue was purified by silica gel column chromatography to afford methyl (2S)-2-(7-chloro-1,1-dioxidobenzo[f][1,2]thiazepin-2(3H)-yl)-3-(6-fluoro-2,3-dimethylphenyl)butanoate (1.36 g, 86.0%).

### Step 5. (2S)-2-(7-chloro-1,1-dioxidobenzo[f][1,2]thiazepin-2(3H)-yl)-3-(6-fluoro-2,3-dimethylphenyl)butanoic acid

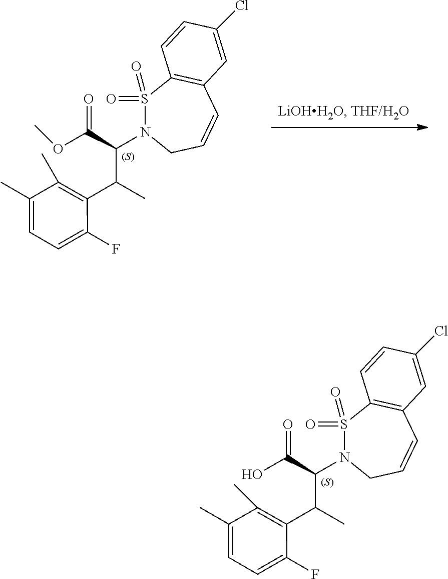

Into a 20 mL vial were added methyl (2S)-2-(7-chloro-1,1-dioxidobenzo[f][1,2]thiazepin-2(3H)-yl)-3-(6-fluoro-2,3-dimethylphenyl)butanoate (300 mg, 0.664 mmol, 1 equiv), LiOH·H₂O (278.56 mg, 6.639 mmol, 3 equiv), THF (9 mL) and H₂O (3 mL) at room temperature. The resulting mixture was stirred overnight at room temperature under air atmosphere. The residue was purified by reverse flash chromatography. This resulted in (2S)-2-(7-chloro-1,1-dioxidobenzo[f][1,2]thiazepin-2(3H)-yl)-3-(6-fluoro-2,3-dimethylphenyl)butanoic acid (315 mg, 32.51%).

### Step 6. 5-((1S)-1-(7-chloro-1,1-dioxidobenzo[f][1,2]thiazepin-2(3H)-yl)-2-(6-fluoro-2,3-dimethylphenyl)propyl)-1,3,4-oxadiazol-2(3H)-one

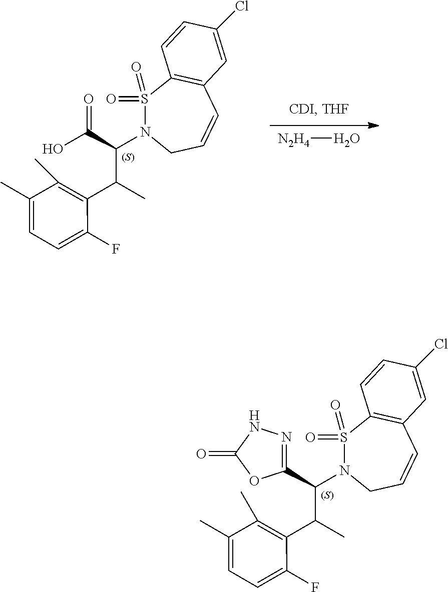

Into a 10 mL vial were added (2S)-2-(7-chloro-1,1-dioxidobenzo[f][1,2]thiazepin-2(3H)-yl)-3-(6-fluoro-2,3-dimethylphenyl)butanoic acid (380 mg, 0.868 mmol, 1 equiv), CDI (365.84 mg, 2.257 mmol, 2.6 equiv) and THF (7.6 mL) at room temperature. The resulting mixture was stirred for 0.5 h at room temperature under air atmosphere. To the above mixture was added hydrazine hydrate (130.32 mg, 2.604 mmol, 3 equiv) in portions at 0° C. The resulting mixture was stirred for additional 1 h at 0° C. The resulting mixture was extracted with EtOAc (2×10 mL). The combined organic layers were washed with brine (1×10 mL), dried over anhydrous Na₂SO₄. After filtration, the filtrate was concentrated under reduced pressure. Into a 25 mL round-bottom flask were added the above crude product and CDI (365.84 mg, 2.257 mmol, 2.6 equiv) in dioxane (0.5 mL) at room temperature. The resulting mixture was stirred for 0.5 h at room temperature under air atmosphere. The resulting mixture was concentrated under reduced pressure. The residue was purified by reverse flash chromatography. This resulted in 5-((1S)-1-(7-chloro-1,1-dioxidobenzo[f][1,2]thiazepin-2(3H)-yl)-2-(6-fluoro-2,3-dimethylphenyl)propyl)-1,3,4-oxadiazol-2(3H)-one (340 mg, 82%).

### Step 7. 5-((1S)-1-(7-chloro-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one

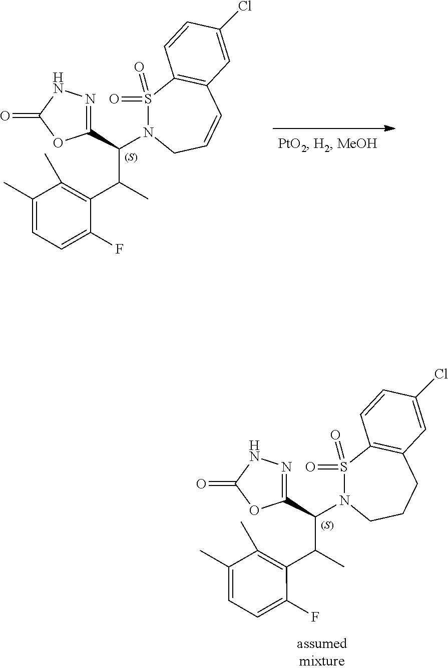

To a stirred solution/mixture of in 5-((1S)-1-(7-chloro-1,1-dioxidobenzo[f][1,2]thiazepin-2(3H)-yl)-2-(6-fluoro-2,3-dimethylphenyl)propyl)-1,3,4-oxadiazol-2(3H)-one (280 mg, 0.586 mmol, 1 equiv) and MeOH (25 mL) was added PtO₂ (200 mg, 0.881 mmol, 1.50 equiv) at room temperature under hydrogen atmosphere. The resulting mixture was stirred for 2 h at room temperature under hydrogen atmosphere. The resulting mixture was filtered, the filter cake was washed with MeOH (2×5 mL). The filtrate was concentrated under reduced pressure. The residue was purified by reverse flash chromatography. This resulted in 5-((1S)-1-(7-chloro-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one (34 mg, 11.9%).

Step 8. 5-((1S,2R)-1-(7-chloro-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one and 5-((1S,2S)-1-(7-chloro-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one

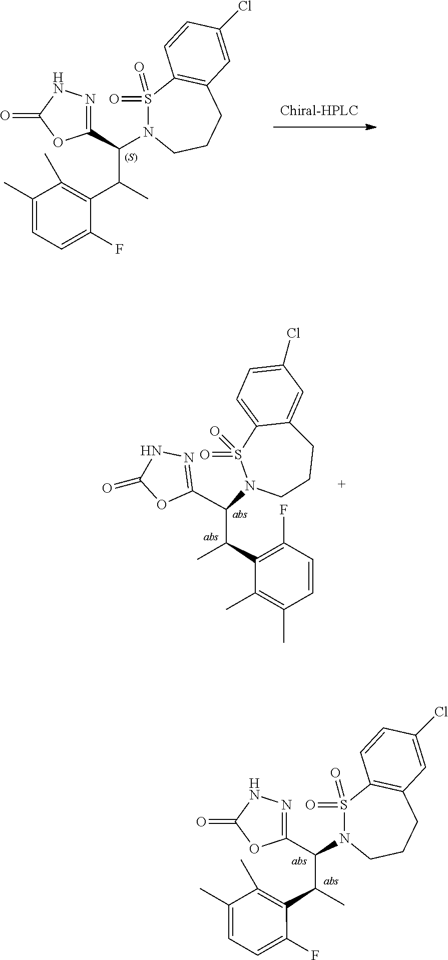

The pure compound of 5-((1S)-1-(7-chloro-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one (95 mg) was purified by chiral chromatography. This resulted in:

First isomer (40.7 mg).

LC-MS: (ES, m/z): [M−H]+=478.00. 1H NMR (400 MHz, DMSO-d6) δ 12.32 (s, 1H), 7.64 (d, J=8.4 Hz, 1H), 7.49-7.31 (m, 2H), 6.94 (dd, J=8.3, 5.8 Hz, 1H), 6.53 (s, 1H), 5.21 (d, J=11.1 Hz, 1H), 3.83 (dd, J=11.5, 6.8 Hz, 1H), 3.50 (d, J=8.2 Hz, 2H), 3.02 (d, J=10.6 Hz, 1H), 2.95 (s, 1H), 2.22 (d, J=15.8 Hz, 6H), 1.52-1.07 (m, 5H).

Second isomer (5.9 mg). LC-MS (ES, m z): [M−H]+=478.05. ¹H NMR (300 MHz, DMSO-d₆) δ 12.32 (s, 1H), 7.64 (d, J=8.3 Hz, 1H), 7.43 (dd, J=8.3, 2.2 Hz, 1H), 7.36 (d, J=2.2 Hz, 1H), 6.94 (t, J=7.0 Hz, 1H), 6.53 (s, 1H), 5.20 (d, J=11.1 Hz, 1H), 3.82 (s, 1H), 3.53 (d, J=16.6 Hz, 2H), 2.98 (dd, J=20.6, 7.5 Hz, 2H), 2.21 (d, J=15.8 Hz, 6H), 1.55-1.08 (m, 5H).

Example 11 and 12: Synthesis of 5-((1S,2S)-1-(7-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one and 5-((1S,2R)-1-(7-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

### Step 1. tert-butyl 2-(2-bromo-4-chlorophenyl) acetate

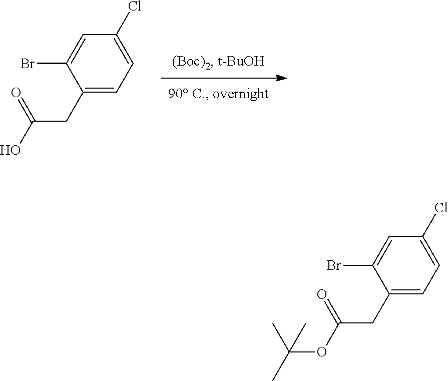 [SMILES: O=C(O)Cc1ccc(Cl)cc1Br ; CC(C)(C)OC(=O)Cc1ccc(Cl)cc1Br]

Into a 250 mL round-bottom flask were added (2-bromo-4-chlorophenyl) acetic acid (5 g, 20.041 mmol, 1 equiv) and t-BuOH (50 mL, 526.152 mmol) at room temperature. To the above mixture was added (Boc)₂O (20 g, 91.638 mmol) in portions at room temperature. The resulting mixture was stirred for additional overnight at 90° C. The reaction was quenched with water. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by silica gel column chromatography to afford tert-butyl 2-(2-bromo-4-chlorophenyl) acetate (6.7 g, 98.46%).

### Step 2. tert-butyl 2-[2-(benzylsulfanyl)-4-chlorophenyl]acetate

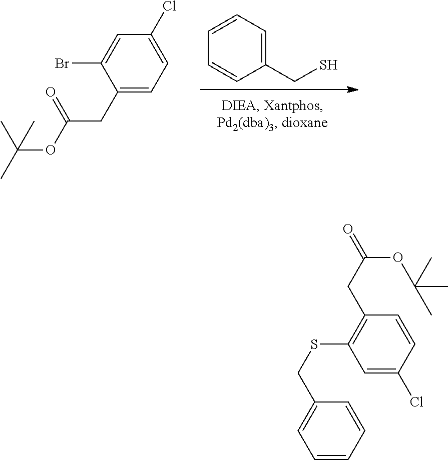

Into a 250 mL round-bottom flask were added tert-butyl 2-(2-bromo-4-chlorophenyl)acetate (6 g, 19.634 mmol, 1 equiv) and dioxane (50 mL, 590.198 mmol) at room temperature. To the above mixture was added DIEA (10.26 mL, 58.902 mmol, 3 equiv) Xantphos (1.14 g, 1.963 mmol, 0.1 equiv), Pd₂(dba)₃ (898.95 mg, 0.982 mmol, 0.05 equiv), benzyl mercaptan (2.77 mL, 23.561 mmol, 1.2 equiv) in portions at room temperature. The resulting mixture was stirred for additional 3 h at 100° C. under nitrogen atmosphere. The reaction was quenched with Water. The resulting mixture was extracted with EtOAc (3×100 mL). The combined organic layers were washed with brine (1×500 mL), dried over anhydrous Na₂SO₄. After filtration, the filtrate was concentrated under reduced pressure. The residue was purified by silica gel column chromatography to afford tert-butyl 2-[2-(benzylsulfanyl)-4-chlorophenyl]acetate (7.3 g, 95.91%).

### Step 3. tert-butyl 2-[4-chloro-2-(chlorosulfonyl)phenyl]acetate

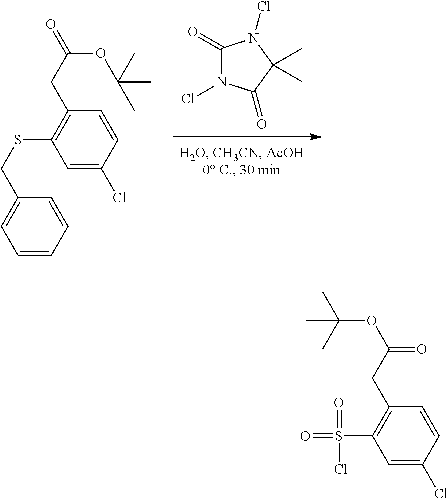

Into a 50 mL 3-necked round-bottom flask were added tert-butyl 2-[2-(benzylsulfanyl)-4-chlorophenyl]acetate (3 g, 8.599 mmol, 1 equiv) and CH₃CN (20 mL) at room temperature. To the above mixture was added H₂O (2 mL), AcOH (3 mL) and 1,3-dichloro-5,5-dimethylimidazolidine-2,4-dione (3.39 g, 17.198 mmol, 2 equiv) in portions at 0° C. under nitrogen atmosphere. The resulting mixture was stirred for additional 30 min at 0° C. The reaction was quenched by the addition of water at 0° C. The resulting mixture was extracted with EtOAc (2×30 mL). The combined organic layers were washed with brine (3×100 mL), dried over anhydrous Na₂SO₄. After filtration, the filtrate was concentrated under reduced pressure. The residue was purified by silica gel column chromatography to afford tert-butyl 2-[4-chloro-2-(chlorosulfonyl) phenyl]acetate (2.24 g, 80.1%).

### Step 4. methyl (2S)-2-((2-(2-(tert-butoxy)-2-oxoethyl)-5-chlorophenyl) sulfonamido)-3-(6-fluoro-2,3-dimethylphenyl) butanoate

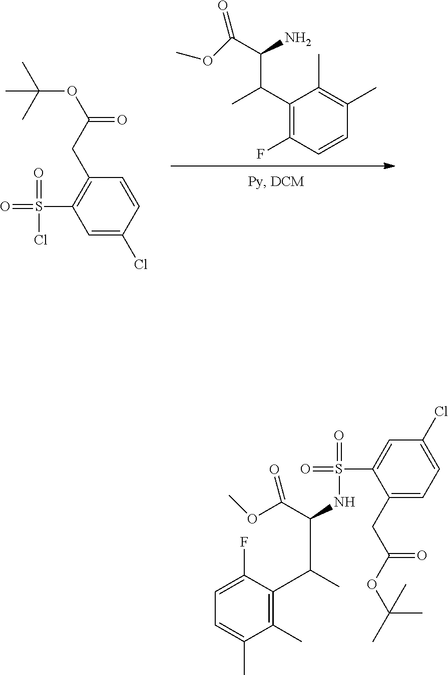

Into a 50 mL round-bottom flask were added methyl (2S)-2-amino-3-(6-fluoro-2,3-dimethylphenyl) butanoate (1.4 g, 5.851 mmol, 1.00 equiv) and DCM (20 mL) at room temperature. To the above mixture was added pyridine (1.39 mL, 17.553 mmol, 3 equiv) and tert-butyl 2-[4-chloro-2-(chlorosulfonyl) phenyl]acetate (2.28 g, 7.021 mmol, 1.2 equiv) dropwise at 0° C. The resulting mixture was stirred for additional 2 h at room temperature. The resulting mixture was concentrated under vacuum. The residue was purified by silica gel column chromatography to afford. methyl (2S)-2-((2-(2-(tert-butoxy)-2-oxoethyl)-5-chlorophenyl) sulfonamido)-3-(6-fluoro-2,3-dimethylphenyl)butanoate (2.64 g, 85.46%).

### Step 5. 2-(4-chloro-2-(N-((2S)-3-(6-fluoro-2,3-dimethylphenyl)-1-methoxy-1-oxobutan-2-yl) sulfamoyl) phenyl)acetic acid

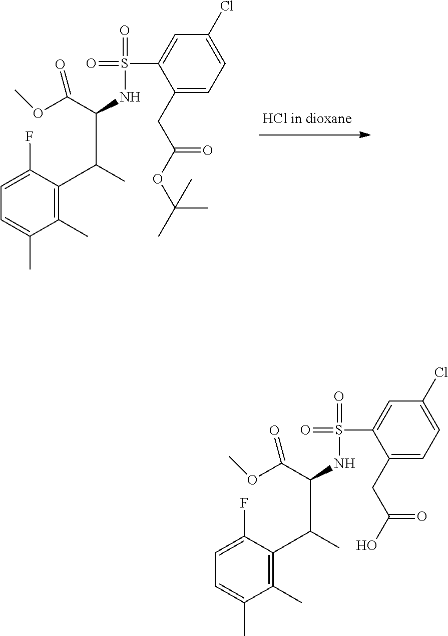 [SMILES: COC(=O)[C@@H](NS(=O)(=O)c1cc(Cl)ccc1CC(=O)OC(C)(C)C)C(C)c1c(F)ccc(C)c1C ; COC(=O)[C@@H](NS(=O)(=O)c1cc(Cl)ccc1CC(=O)O)C(C)c1c(F)ccc(C)c1C]

Into a 100 mL round-bottom flask were added methyl (2S)-2-{2-[2-(tert-butoxy)-2-oxoethyl]-5-chlorobenzenesulfonamido}-3-(6-fluoro-2,3-dimethylphenyl) butanoate (2.64 g, 5.000 mmol, 1 equiv) and HCl 4N in 1,4-dioxane (5 mL) at room temperature. The resulting mixture was stirred for 2 h at 50° C. The resulting mixture was concentrated under reduced pressure. The residue was purified by silica gel column chromatography to afford 2-(4-chloro-2-(N-((2S)-3-(6-fluoro-2,3-dimethylphenyl)-1-methoxy-1-oxobutan-2-yl) sulfamoyl) phenyl) acetic acid (2.25 g, 95.36%).

### Step 6. methyl (2S)-2-[5-chloro-2-(2-hydroxyethyl) benzenesulfonamido]-3-(6-fluoro-2,3-dimethylphenyl) butanoate

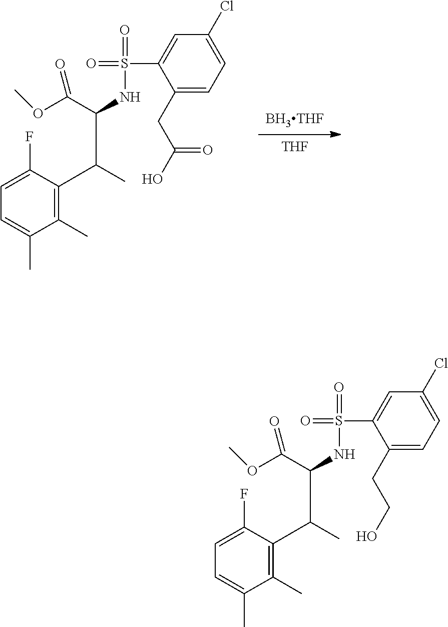 [SMILES: COC(=O)[C@@H](NS(=O)(=O)c1cc(Cl)ccc1CC(=O)O)C(C)c1c(F)ccc(C)c1C ; COC(=O)[C@@H](NS(=O)(=O)c1cc(Cl)ccc1CCO)C(C)c1c(F)ccc(C)c1C ; "BH3·THF" ; "THF"]

To a stirred solution of BH₃·THF (10.17 mL, 10.172 mmol, 2 equiv) were added 2-(4-chloro-2-(N-((2S)-3-(6-fluoro-2,3-dimethylphenyl)-1-methoxy-1-oxobutan-2-yl) sulfamoyl) phenyl) acetic acid (2.4 g, 5.086 mmol, 1 equiv) in tetrahydrofuran (20 mL) dropwise over 10 min at room temperature under nitrogen atmosphere. The reaction was quenched with MeOH at room temperature. The resulting mixture was concentrated under vacuum. The residue was purified by reverse flash chromatography. This resulted in methyl (2S)-2-[5-chloro-2-(2-hydroxyethyl) benzenesulfonamido]-3-(6-fluoro-2,3-dimethylphenyl)butanoate (1.18 g, 50.67%).

### Step 7. methyl (2S)-2-((5-chloro-2-(2-((methylsulfonyl)oxy)ethyl)phenyl)sulfonamido)-3-(6-fluoro-2,3-dimethylphenyl)butanoate

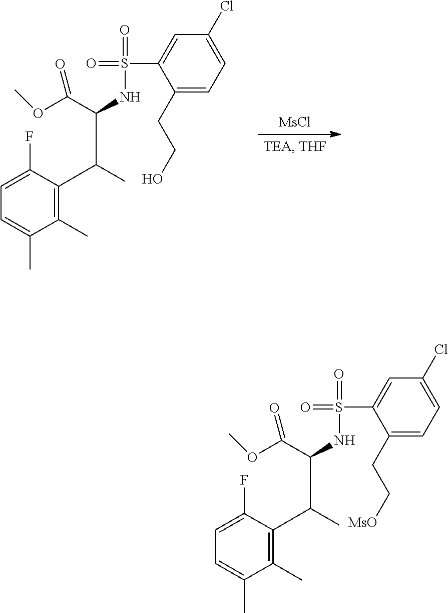 [SMILES: "MsCl" ; COC(=O)[C@@H](NS(=O)(=O)c1cc(Cl)ccc1CCO)C(C)c1c(F)ccc(C)c1C ; "TEA, THF" ; COC(=O)[C@@H](NS(=O)(=O)c1cc(Cl)ccc1CCOS(C)(=O)=O)C(C)c1c(F)ccc(C)c1C]

Into a 100 mL round-bottom flask were added methyl (2S)-2-[5-chloro-2-(2-hydroxyethyl) benzenesulfonamido]-3-(6-fluoro-2,3-dimethylphenyl) butanoate (1.18 g, 2.577 mmol, 1 equiv) and DCM (10 mL) at room temperature. To the above mixture was added TEA (2.15 mL, 15.462 mmol, 6 equiv) and methanesulfonyl chloride (2.58 mL, 5.154 mmol, 2 equiv) dropwise at 0° C. The resulting mixture was stirred for additional 1 h at 0° C. The reaction was quenched with water at room temperature. The resulting mixture was extracted with CH₂Cl₂ (2×20 mL). The combined organic layers were washed with brine (1×60 mL), dried over anhydrous MgSO₄. After filtration, the filtrate was concentrated under reduced pressure. The residue was purified by silica gel column chromatography to afford methyl (2S)-2-{5-chloro-2-[2-(methanesulfonyloxy) ethyl]benzenesulfonamido}-3-(6-fluoro-2,3-dimethylphenyl)butanoate (870 mg, 63%).

### Step 8. methyl (2S)-2-(7-chloro-1,1-dioxido-3,4-dihydro-2H-benzo[e][1,2]thiazin-2-yl)-3-(6-fluoro-2,3-dimethylphenyl)butanoate

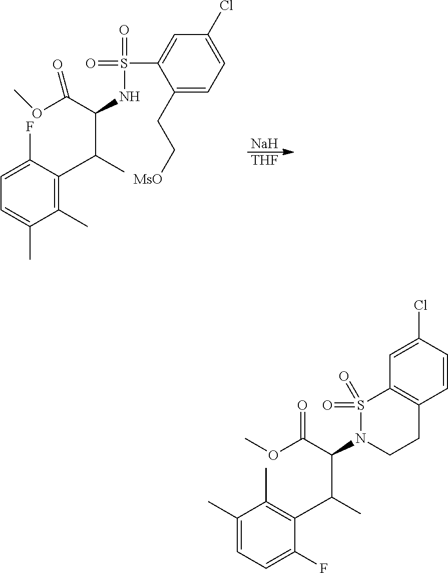

Into a 50 mL 3-necked round-bottom flask were added methyl (2S)-2-{5-chloro-2-[2-(methanesulfonyloxy) ethyl]benzenesulfonamido}-3-(6-fluoro-2,3-dimethylphenyl)butanoate (870 mg, 1.623 mmol, 1 equiv) and tetrahydrofuran (10 mL) at room temperature. To the above mixture was added NaH (58.42 mg, 2.434 mmol, 1.5 equiv) in portions at 0° C. The resulting mixture was stirred for additional overnight at room temperature. The reaction was quenched by the addition of sat. NH₄Cl (aq.) (10 mL) at room temperature. The resulting mixture was extracted with EtOAc (2×20 mL). The combined organic layers were washed with brine (1×60 mL), dried over anhydrous Na₂SO₄. After filtration, the filtrate was concentrated under reduced pressure. The residue was purified by silica gel column chromatography to afford methyl (2S)-2-(7-chloro-1,1-dioxido-3,4-dihydro-2H-benzo[e][1,2]thiazin-2-yl)-3-(6-fluoro-2,3-dimethylphenyl)butanoate (560 mg, 78.4%).

### Step 9. (2S)-2-(7-chloro-1,1-dioxido-3,4-dihydro-2H-benzo[e][1,2]thiazin-2-yl)-3-(6-fluoro-2,3-dimethylphenyl)butanoic acid

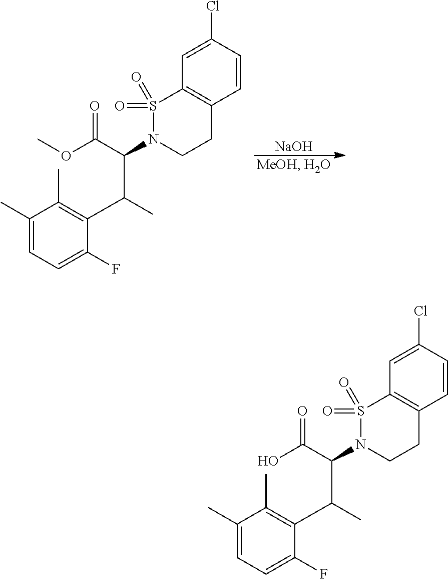

To a stirred mixture of methyl (2S)-2-(7-chloro-1,1-dioxido-3,4-dihydro-2H-benzo[e][1,2]thiazin-2-yl)-3-(6-fluoro-2,3-dimethylphenyl)butanoate (550 mg, 1.250 mmol, 1 equiv) in MeOH (10 mL, 24.699 mmol) were added NaOH (100.01 mg, 2.500 mmol, 2 equiv) and H₂O (2 mL, 11.102 mmol) dropwise at room temperature. The resulting mixture was stirred for overnight at 60° C. under. The mixture was acidified to pH 6 with HCl (2M). The residue was purified by reverse flash chromatography. This resulted in (2S)-2-(7-chloro-1,1-dioxido-3,4-dihydro-2H-benzo[e][1,2]thiazin-2-yl)-3-(6-fluoro-2,3-dimethylphenyl)butanoic acid (510 mg, 95.78%).

### Step 10. tert-butyl 2-((2S)-2-(7-chloro-1,1-dioxido-3,4-dihydro-2H-benzo[e][1,2]thiazin-2-yl)-3-(6-fluoro-2,3-dimethylphenyl)butanoyl)hydrazine-1-carboxylate

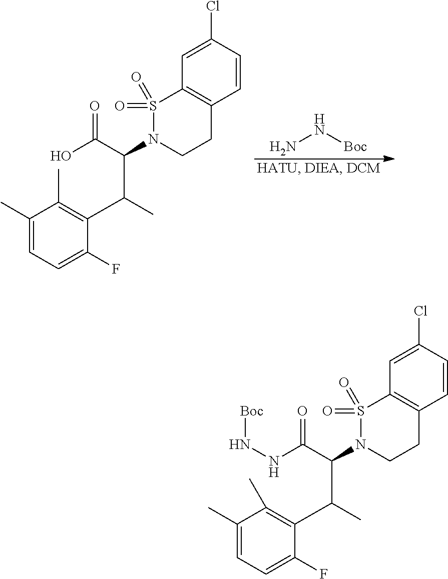

Into a 50 mL round-bottom flask were added (2S)-2-(7-chloro-1,1-dioxido-3,4-dihydro-2H-benzo[e][1,2]thiazin-2-yl)-3-(6-fluoro-2,3-dimethylphenyl)butanoic acid (500 mg, 1.174 mmol, 1 equiv) and DCM (5 mL) at room temperature. To the above mixture was added HATU (669.58 mg, 1.761 mmol, 1.5 equiv) DIEA (613.48 μL, 3.522 mmol, 3 equiv) and tert-butoxycarbohydrazide (232.74 mg, 1.761 mmol, 1.5 equiv) in portions at room temperature. The resulting mixture was stirred for additional 1 h at room temperature. The resulting mixture was concentrated under vacuum. The residue was purified by silica gel column chromatography to afford. tert-butyl 2-((2S)-2-(7-chloro-1,1-dioxido-3,4-dihydro-2H-benzo[e][1,2]thiazin-2-yl)-3-(6-fluoro-2,3-dimethylphenyl)butanoyl)hydrazine-1-carboxylate (520 mg, 82.0%).

### Step 11. (2S)-2-(7-chloro-1,1-dioxido-3,4-dihydro-2H-benzo[e][1,2]thiazin-2-yl)-3-(6-fluoro-2,3-dimethylphenyl)butanehydrazide

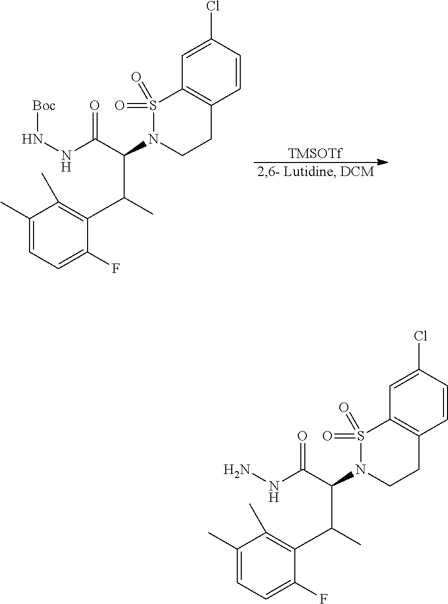 [SMILES: "TMSOTf" ; Cc1ccc(F)c(C(C)[C@@H](C(=O)NNC(=O)OC(C)(C)C)N2CCc3ccc(Cl)cc3S2(=O)=O)c1C ; "2,6- Lutidine, DCM" ; Cc1ccc(F)c(C(C)[C@@H](C(=O)NN)N2CCc3ccc(Cl)cc3S2(=O)=O)c1C]

Into a 50 mL round-bottom flask were added tert-butyl 2-((2S)-2-(7-chloro-1,1-dioxido-3,4-dihydro-2H-benzo[e][1,2]thiazin-2-yl)-3-(6-fluoro-2,3-dimethylphenyl)butanoyl)hydrazine-1-carboxylate (520 mg, 0.963 mmol, 1 equiv) and DCM (20 mL) at room temperature. To the above mixture was added 2,6-Lutidine (2063.55 mg, 19.260 mmol, 20 equiv). To the mixture was added trimethylsilyl triflate (3423.98 mg, 15.408 mmol, 16 equiv) dropwise at 0° C. The resulting mixture was stirred for additional 1 h at room temperature. The resulting mixture was concentrated under reduced pressure. The residue was purified by reverse flash chromatography. This resulted in (2S)-2-(7-chloro-1,1-dioxido-3,4-dihydro-2H-benzo[e][1,2]thiazin-2-yl)-3-(6-fluoro-2,3-dimethylphenyl)butanehydrazide (290 mg, 68.5%).

### Step 12. 5-((1S)-1-(7-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

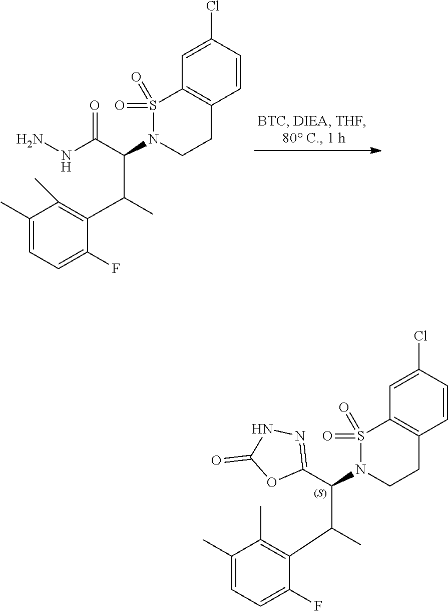

To a stirred mixture of (2S)-2-(7-chloro-1,1-dioxido-3,4-dihydro-2H-benzo[e][1,2]thiazin-2-yl)-3-(6-fluoro-2,3-dimethylphenyl)butanehydrazide (280 mg, 0.636 mmol, 1 equiv) in THF (3 mL) were added DIEA (277.16 μL, 1.590 mmol, 2.5 equiv) and Triphosgene (94.43 mg, 0.318 mmol, 0.5 equiv) dropwise at room temperature. The resulting mixture was stirred for 1 h at 80° C. The resulting mixture was concentrated under reduced pressure. The residue was purified by reverse flash chromatography. This resulted in 5-((1S)-1-(7-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (270 mg, 91.05%).

Step 13. 5-((1S,2S)-1-(7-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one and 5-((1S,2R)-1-(7-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

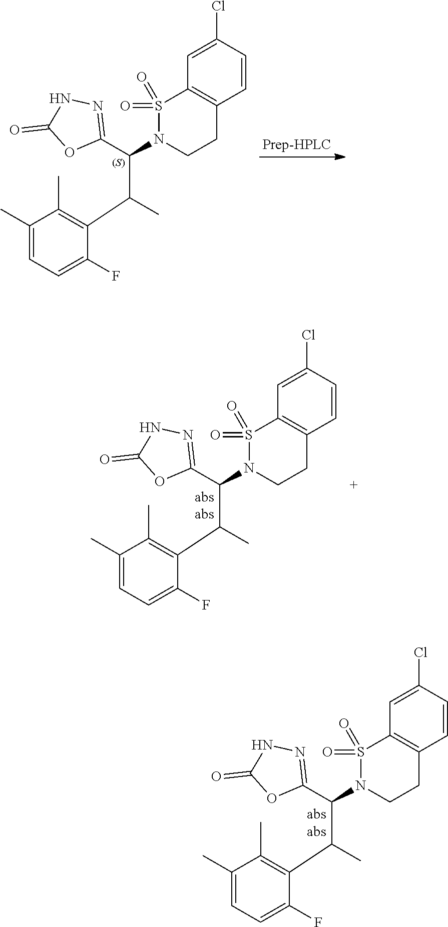

The crude product (270 mg) was purified by Prep-HPLC to afford:

First isomer: (39.7 mg, 14.56%). LC-MS (ES, m/z):M−H=464.05. ¹H NMR (300 MHz, Methanol-d₄) δ 7.51 (d, J=2.2 Hz, 1H), 7.44 (dd, J=8.2, 2.2 Hz, 1H), 7.18 (d, J=8.3 Hz, 1H), 7.00 (dd, J=8.4, 5.7 Hz, 1H), 6.73 (dd, J=11.9, 8.3 Hz, 1H), 5.46 (d, J=11.3 Hz, 1H), 3.90 (dq, J=13.1, 6.7 Hz, 1H), 3.69 (dt, J=14.0, 7.2 Hz, 1H), 3.52 (ddd, J=13.4, 7.6, 5.8 Hz, 1H), 2.88 (dt, J=16.8, 6.5 Hz, 1H), 2.82-2.66 (m, 1H), 2.29 (s, 3H), 2.22 (s, 3H), 1.40-1.19 (m, 3H).

Second isomer: (32.4 mg, 12.01%). LC-MS (ES, m/z): M−H=464.05. 1H NMR (300 MHz, Methanol-d₄) δ 7.74 (d, J=2.2 Hz, 1H), 7.51 (dd, J=8.3, 2.2 Hz, 1H), 7.30 (d, J=8.3 Hz, 1H), 7.00 (dd, J=8.5, 5.8 Hz, 1H), 6.74 (dd, J=12.0, 8.4 Hz, 1H), 5.52 (dd, J=11.7, 1.7 Hz, 1H), 4.18-3.75 (m, 3H), 3.23-2.87 (m, 2), 2.31 (s, 3H), 2.21 (s, 3H), 1.42 (dd, J=6.9, 1.2 Hz, 3H).

### Example 13: 6-chloro-2-((1S,2R)-2-(6-fluoro-2,3-dimethylphenyl)-1-(2H-tetrazol-5-yl)propyl)-3,4-dihydro-2H-benzo[e][1,2]thiazine 1,1-dioxide

### Step 1. tert-butyl N-[(1S,2R)-1-carbamoyl-2-(6-fluoro-2,3-dimethylphenyl)propyl]carbamate

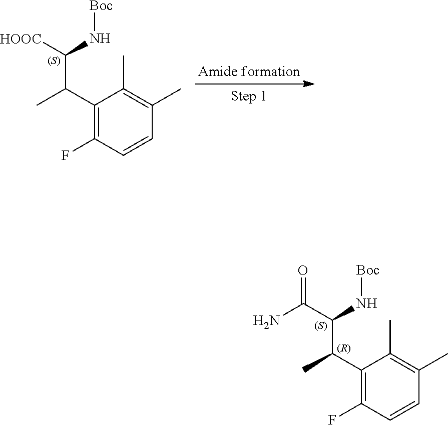

A solution of (2S)-2-{[(tert-butoxy)carbonyl]amino}-3-(6-fluoro-2,3-dimethylphenyl)butanoic acid (1.0 g, 3.073 mmol, 1.0 eq), triethylamine (471 μL, 3.379 mmol, 1.1 eq) and ethyl chloroformate (323 μL, 3.378 mmol, 1.099 eq) in THF (50 mL) was cooled to −10° C. After 1 h, ammonia solution (25% in H₂O, 25 mL) was added dropwise and the reaction was continued overnight. Solvents were removed in vacuo and the residue was taken up in EtOAc. Organic layer was washed with 1M Na₂HPO₄, water and brine, dried over Na₂SO₄, filtered and evaporated to dryness. The residue was purified by FCC to afford tert-butyl N-[(1S,2R)-1-carbamoyl-2-(6-fluoro-2,3-dimethylphenyl)propyl]carbamate as a single diastereoisomer (550 mg, 1.695 mmol, yield 52%). LC-MS: m/z=325.0 [M+H]+. 1H NMR (300 MHz, DMSO-d₆) 7.02-6.91 (m, 3H), 6.80 (dd, J=11.6, 8.3 Hz, 1H), 6.70 (s, 1H), 4.32 (t, J=9.9 Hz, 1H), 3.42 (t, J=8.5 Hz, 1H), 2.18 (s, 6H), 1.40 (s, 9H), 1.22-1.14 (m, 3H).

### Step 2. tert-butyl N-[(1S,2R)-1-cyano-2-(6-fluoro-2,3-dimethylphenyl)propyl]carbamate

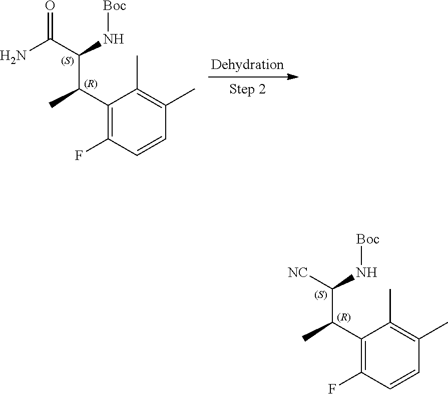

Trifluoroacetic anhydride (354 μL, 2.547 mmol, 1.502 eq) was added dropwise at 0° C. to a solution of tert-butyl N—[(1S,2R)-1-carbamoyl-2-(6-fluoro-2,3-dimethylphenyl) propyl]carbamate (550 mg, 1.695 mmol, 1.0 eq) in pyridine (16.5 mL). The reaction was continued at RT overnight. Solvent was removed in vacuo. The residue was purified by FCC to afford tert-butyl N—[(JS,2R)-1-cyano-2-(6-fluoro-2,3-dimethylphenyl) propyl]carbamate (400 mg, 1.306 mmol, yield 76%). ¹H NMR (300 MHz, DMSO-d₆) 7.94 (d, J=8.8 Hz, 1H), 7.14 (dd, J=8.4, 5.9 Hz, 1H), 6.95 (dd, J=12.0, 8.4 Hz, 1H), 4.78 (t, J=10.1 Hz, 1H), 3.63 (dd, J=11.7, 7.0 Hz, 1H), 2.25 (s, 6H), 1.44 (s, 9H), 1.21 (d, J=6.9 Hz, 3H).

### Step 3. tert-butyl N-[(1S,2R)-2-(6-fluoro-2,3-dimethylphenyl)-1-(2H-1,2,3,4-tetrazol-5-yl)propyl]carbamate

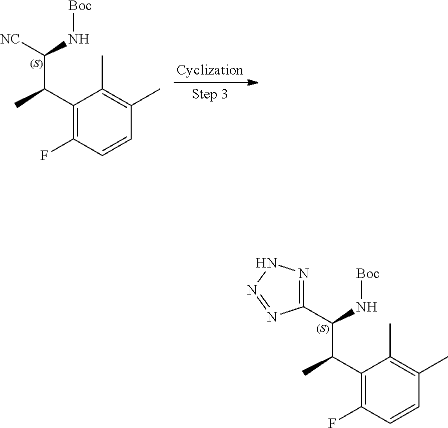

To a solution of tert-butyl N—[(1S,2R)-1-cyano-2-(6-fluoro-2,3-dimethylphenyl) propyl]carbamate (400 mg, 1.306 mmol, 1.0 eq) in DMF (20.0 mL) sodium azide (127 mg, 1.954 mmol, 4 eq) was added followed by NH₄Cl (279 mg, 5.216 mmol, 4 eq). The reaction was carried out at 110° C. overnight. The mixture was cooled down, diluted with EtOAc, and washed twice with water, 10% sol. of NaH₂PO₄, brine, dried, filtered and concentrated to afford tert-butyl N-[(1S,2R)-2-(6-fluoro-2,3-dimethylphenyl)-1-(2H-1,2,3,4-tetrazol-5-yl)propyl]carbamate (360 mg, 1.03 mmol, yield 75%). LC-MS: m/z=349.95 [M+H]+. ¹H NMR (300 MHz, DMSO-d₆) 7.79 (d, J=8.6 Hz, 1H), 6.91 (dd, J=8.4, 5.8 Hz, 1H), 6.75 (dd, J=11.8, 8.4 Hz, 1H), 5.41 (t, J=9.9 Hz, 1H), 3.82 (q, J=8.1, 6.6 Hz, 1H), 2.09 (d, J=5.5 Hz, 6H), 1.36 (d, J=9.2 Hz, 12H).

### Step 4. (1S,2R)-2-(6-fluoro-2,3-dimethylphenyl)-1-(2H-1,2,3,4-tetrazol-5-yl)propan-1-amine

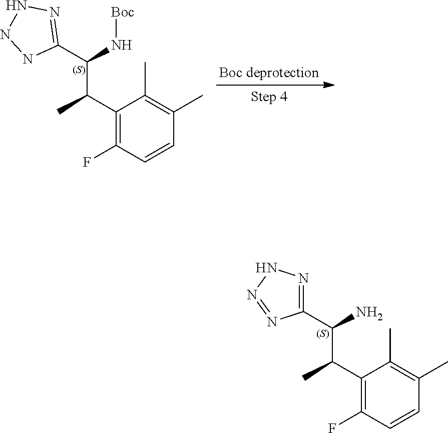

A mixture of tert-butyl N-[(1S,2R)-2-(6-fluoro-2,3-dimethylphenyl)-1-(2H-1,2,3,4-tetrazol-5-yl)propyl]carbamate (360 mg, 1.03 mmol, 1.0 eq) and 4M HCl in dioxane (5.152 mL, 20.608 mmol, 20 eq) was stirred at rt for 2 h until full conversion was observed. Solvents were removed in vacuo, then the residue was co-evaporated twice with toluene and dried under high vacuum to afford (1S,2R)-2-(6-fluoro-2,3-dimethylphenyl)-1-(2H-1,2,3,4-tetrazol-5-yl)propan-1-amine as a hydrochloride (290 mg, 1.02 mmol, yield 100%). LC-MS: m/z=349.95 [M+H]+. ¹H NMR (300 MHz, DMSO-d₆) 9.16 (s, 3H), 6.97 (dd, J=8.4, 5.9 Hz, 1H), 6.82 (dd, J=11.8, 8.4 Hz, 1H), 5.06 (d, J=10.8 Hz, 1H), 3.99 (d, J=18.4 Hz, 1H), 2.05 (d, J 10.5 Hz, 6H), 1.55-1.42 (m, 3H).

### Step 5. Methyl 2-(2-bromo-5-chlorophenyl) acetate

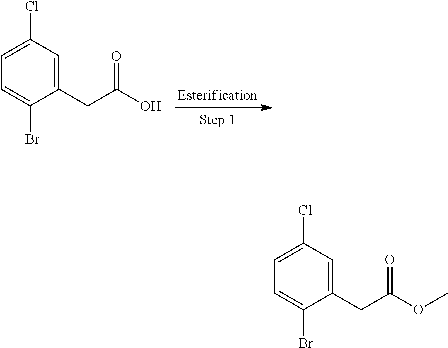

To a cooled solution of 2-Bromo-5-chlorophenylacetic acid (2.0 g, 8.016 mmol, 1.0 eq) in MeOH (40 mL) thionyl chloride (2.339 mL, 32.069 mmol, 4.0 eq) was added dropwise. The mixture was allowed to warm to RT and stirred for 2 h. After the reaction was completed, the volatiles were removed under reduced pressure. The residue was suspended in sat. NaHCO3 and extracted with DCM. The combined extracts were washed with brine, dried over Na₂SO₄, filtered, and evaporated under reduced pressure to afford methyl 2-(2-bromo-5-chlorophenyl) acetate as a colorless liquid (2.11 g, yield 100%). 1H NMR (300 MHz, Chloroform-d) δ 7.49 (d, J=8.5 Hz, 1H), 7.29 (d, J=2.5 Hz, 1H), 7.14 (dd, J=8.5, 2.5 Hz, 1H), 3.76 (s, 2H), 3.73 (s, 3H).

### Step 6. Methyl 2-[2-(benzylsulfanyl)-5-chlorophenyl]acetate

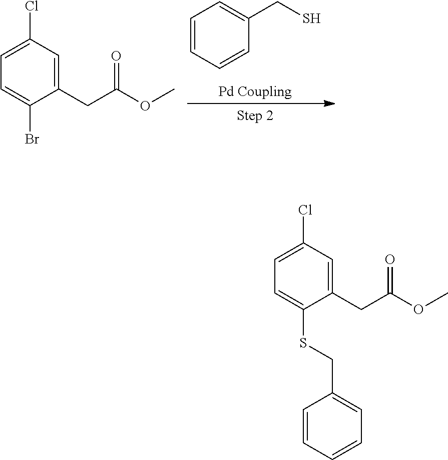

To a solution of methyl 2-(2-bromo-5-chlorophenyl)acetate (1.91 g, 7.248 mmol, 1.0 eq) in dioxane (19 mL) were added DIPEA (2.525 mL, 14.497 mmol, 2.0 eq), tris(dibenzylideneacetone)dipalladium(0) [Pd2(dba)3] (332 mg, 0.363 mmol, 0.05 eq) and Xantphos (294 mg, 0.508 mmol, 0.07 eq). The mixture was thoroughly degassed and purged with argon with constant stirring for 15 min. Afterwards, the reaction vessel was immersed in a preheated oil bath to 110° C. and benzyl mercaptan (0.817 mL, 6.96 mmol, 0.96 eq) was added via a syringe. The reaction vessel was sealed, and the reaction was continued for 20 h. Upon completion, the mixture was allowed to cool to room temperature. The volatiles were removed in vacuo. The residue was redissolved in EtOAc and filtered through a pad of silica gel. The filtrate was concentrated and purified by FCC to give methyl 2-[2-(benzylsulfanyl)-5-chlorophenyl]acetate (2.18 g, 7.106 mmol, yield 98%). 1H NMR (300 MHz, DMSO-d6) δ 7.44 (d, J=8.4 Hz, 1H), 7.38 (d, J=2.3 Hz, 1H), 7.34-7.20 (m, 6H), 4.16 (s, 2H), 3.72 (s, 2H), 3.60 (s, 3H).

### Step 7. Methyl 2-[5-chloro-2-(chlorosulfonyl)phenyl]acetate

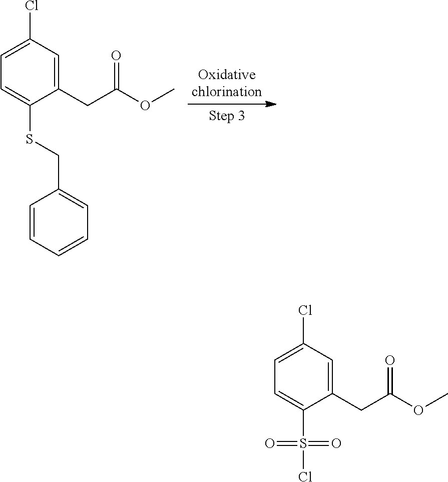 [SMILES: COC(=O)Cc1cc(Cl)ccc1SCc1ccccc1 ; COC(=O)Cc1cc(Cl)ccc1S(=O)(=O)Cl]

To a cooled solution of methyl 2-[2-(benzylsulfanyl)-5-chlorophenyl]acetate (530 mg, 1.728 mmol, 1.0 eq) in glacial acetic acid (7.95 mL) and water (1.6 mL), N-chlorosuccinimide (923 mg, 6.912 mmol, 4.0 eq) was added portion wise. The mixture was stirred at 0° C. for 20 min, cooling bath was removed, and the reaction was continued at RT for 30 min. Upon completion, the reaction mixture was diluted with Et2O and washed with water six times. Organic layer was dried, filtered, and concentrated. The residue was purified by FCC to give methyl 2-[5-chloro-2-(chlorosulfonyl) phenyl]acetate (488 mg, 1.724 mmol, yield 100%). 1H NMR (300 MHz, Chloroform-d) δ 8.06 (d, J=8.4 Hz, 1H), 7.56-7.45 (m, 2H), 4.15 (s, 2H), 3.75 (s, 3H).

### Step 8. methyl 2-(5-chloro-2-(N-((1S,2R)-2-(6-fluoro-2,3-dimethylphenyl)-1-(2H-tetrazol-5-yl)propyl)sulfamoyl)phenyl)acetate

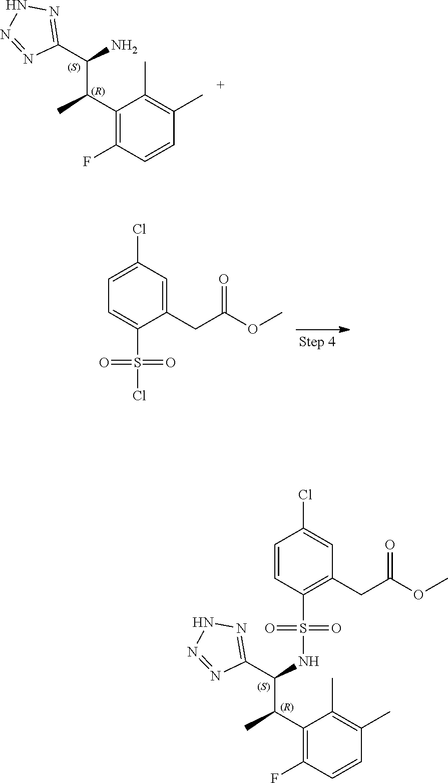 [SMILES: Cc1ccc(F)c([C@@H](C)[C@H](N)c2nn[nH]n2)c1C ; COC(=O)Cc1cc(Cl)ccc1S(=O)(=O)Cl ; COC(=O)Cc1cc(Cl)ccc1S(=O)(=O)N[C@H](c1nn[nH]n1)[C@H](C)c1c(F)ccc(C)c1C]

To a solution of (1S,2R)-2-(6-fluoro-2,3-dimethylphenyl)-1-(2H-1,2,3,4-tetrazol-5-yl)propan-1-amine (493 mg, 1.725 mmol, 1.0 eq) in pyridine (5 mL) was added dropwise methyl 2-[5-chloro-2-(chlorosulfonyl)phenyl]acetate (0.488 g, 1.724 mmol, 1.0 eq) as a DCM solution (5 mL) and the mixture was stirred at rt overnight. Pyridine was removed under reduced pressure and the solid residue was purified by FCC to give methyl 2-(5-chloro-2-(N-((1S,2R)-2-(6-fluoro-2,3-dimethylphenyl)-1-(2H-tetrazol-5-yl)propyl)sulfamoyl)phenyl)acetate (655 mg, 1.321 mmol, yield 77%).

LC-MS: m/z=494.08 [M−H]—. 1H NMR (300 MHz, DMSO-d6) δ 8.38 (s, 1H), 7.50 (d, J=8.5 Hz, 1H), 7.36 (d, J=2.2 Hz, 1H), 7.28 (dd, J=8.5, 2.2 Hz, 1H), 6.80 (dd, J=8.4, 5.7 Hz, 1H), 6.63 (dd, J=11.8, 8.3 Hz, 1H), 5.02 (d, J=10.9 Hz, 1H), 3.96 (d, J=3.3 Hz, 2H), 3.79-3.64 (m, 1H), 3.59 (s, 3H), 2.03 (s, 3H), 2.02 (s, 3H), 1.19 (d, J=7.0 Hz, 3H).

### Step 9. 2-(5-chloro-2-(N-((1S,2R)-2-(6-fluoro-2,3-dimethylphenyl)-1-(2H-tetrazol-5-yl)propyl)sulfamoyl)phenyl)acetic acid

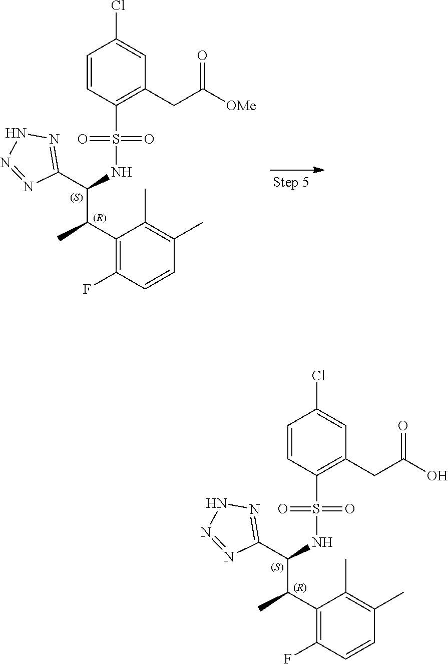 [SMILES: COC(=O)Cc1cc(Cl)ccc1S(=O)(=O)N[C@H](c1nn[nH]n1)[C@H](C)c1c(F)ccc(C)c1C ; Cc1ccc(F)c([C@@H](C)[C@H](NS(=O)(=O)c2ccc(Cl)cc2CC(=O)O)c2nn[nH]n2)c1C]

To a stirred solution of methyl 2-(5-chloro-2-(N-((1S,2R)-2-(6-fluoro-2,3-dimethylphenyl)-1-(2H-tetrazol-5-yl)propyl)sulfamoyl)phenyl)acetate (350 mg, 0.706 mmol, 1.0 eq) in THF (7.0 mL) and water (1.75 mL), lithium hydroxide monohydrate (65 mg, 1.549 mmol, 2.2 eq) was added. The reaction was carried out at rt overnight. The mixture was acidified with 1M HCl to pH 1-2 and extracted with DCM. Organic layers were combined, washed with brine, dried over Na₂SO₄, filtered, and concentrated in vacuo. 2-(5-chloro-2-(N-((1S,2R)-2-(6-fluoro-2,3-dimethylphenyl)-1-(2H-tetrazol-5-yl)propyl) sulfamoyl)phenyl)acetic acid (340 mg, yield 100%) was used in the next step without further purification. LC-MS: m/z=480.12 [M−H]—. 1H NMR (300 MHz, DMSO-d6) δ 8.32 (s, 1H), 7.45 (d, J=8.5 Hz, 1H), 7.29 (d, J=2.2 Hz, 1H), 7.15 (dd, J=8.5, 2.2 Hz, 1H), 6.80 (dd, J=8.4, 5.6 Hz, 1H), 6.65 (dd, J=11.7, 8.2 Hz, 1H), 5.15 (d, J=11.1 Hz, 1H), 3.93 (d, J=15.5 Hz, 1H), 3.76-3.65 (m, 2H), 2.01 (s, 3H), 2.00 (s, 3H), 1.30 (d, J=6.9 Hz, 3H).

### Step 10. 4-chloro-N-[(1S,2R)-2-(6-fluoro-2,3-dimethylphenyl)-1-(2H-1,2,3,4-tetrazol-5-yl) propyl]-2-(2-hydroxyethyl)benzene-1-sulfonamide

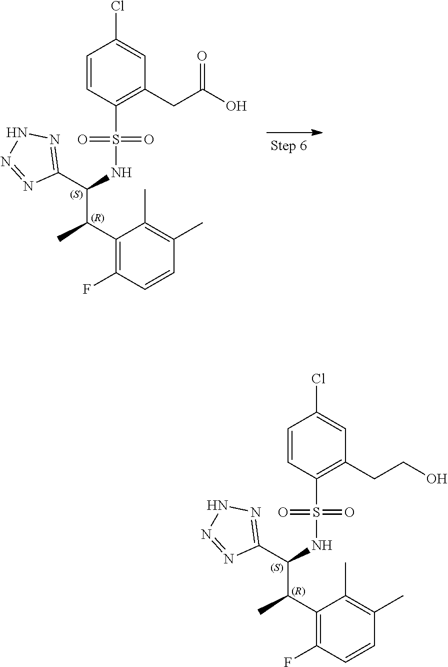

To a solution of 2-(5-chloro-2-(N-((1S,2R)-2-(6-fluoro-2,3-dimethylphenyl)-1-(2H-tetrazol-5-yl) propyl) sulfamoyl)phenyl)acetic acid (125 mg, 0.259 mmol, 1.0 eq) in THF (6.25 mL) was added borane dimethyl sulfide complex (0.246 mL, 2.594 mmol, 10 eq). The reaction was continued at rt overnight. Upon completion, the mixture was cooled to 0° C. and slowly quenched with MeOH. The volatiles were removed under reduced pressure and the residue purified by FCC to give 4-chloro-N-[(1S,2R)-2-(6-fluoro-2,3-dimethylphenyl)-1-(2H-1,2,3,4-tetrazol-5-yl) propyl]-2-(2-hydroxyethyl) benzene-1-sulfonamide (80 mg, 0.171 mmol, yield 66%). LC-MS: m/z=468.3, 470.2 [M+H]+; 466.4, 468.4 [M−H]−

### Step 11. 5-chloro-2-(N-((1S,2R)-2-(6-fluoro-2,3-dimethylphenyl)-1-(2H-tetrazol-5-yl) propyl) sulfamoyl) phenethyl methanesulfonate

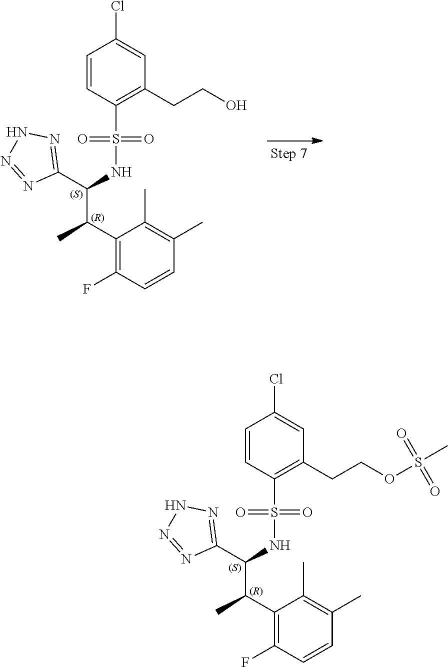

To a solution of 4-chloro-N-[(1S,2R)-2-(6-fluoro-2,3-dimethylphenyl)-1-(2H-1,2,3,4-tetrazol-5-yl)propyl]-2-(2-hydroxyethyl)benzene-1-sulfonamide (80 mg, 0.171 mmol, 1.0 eq) in DCM (4.8 mL), triethylamine (0.071 mL, 0.509 mmol, 3 eq) and methanesulfonyl chloride (0.026 mL, 0.336 mmol, 1.965 eq) were added at 0° C. The mixture was stirred at 0° C. for 2 h, then cooling bath was removed and the reaction was continued at RT for 2 h. The reaction was quenched with water and extracted with DCM. Organic layers were combined, dried over Na₂SO₄, filtered, and concentrated under vacuum to dryness. Crude 5-chloro-2-(N-((1S,2R)-2-(6-fluoro-2,3-dimethylphenyl)-1-(2H-tetrazol-5-yl) propyl) sulfamoyl) phenethyl methanesulfonate (96 mg, yield 100%) was used in the next step without purification. LC-MS: m/z=546.4, 548.3 [M+H]+

### Step 12. 6-chloro-2-((1S,2R)-2-(6-fluoro-2,3-dimethylphenyl)-1-(2H-tetrazol-5-yl)propyl)-3,4-dihydro-2H-benzo[e][1,2]thiazine 1,1-dioxide

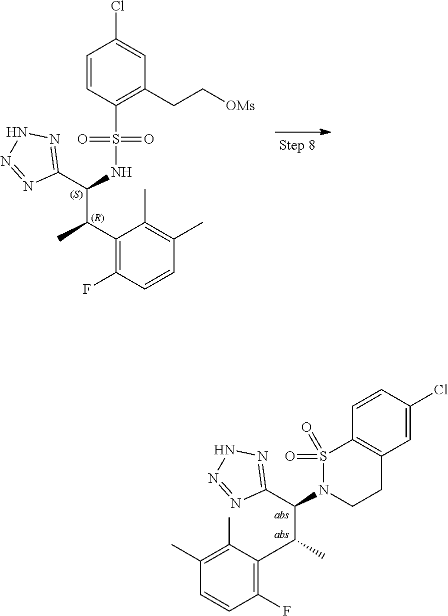

To solution of 5-chloro-2-(N-((1S,2R)-2-(6-fluoro-2,3-dimethylphenyl)-1-(2H-tetrazol-5-yl) propyl)sulfamoyl)phenethyl methanesulfonate (96 mg, 0.176 mmol, 1.0 eq) in THF (9.6 mL) was added sodium hydride (60% in mineral oil, 15 mg, 0.352 mmol, 2 eq). The reaction was continued at RT for 2 h. The mixture was poured into 10% aqueous NaH₂PO₄ and extracted with DCM. Combined organic layers were dried over Na₂SO₄, filtered and concentrated. The residue was purified by pHPLC to afford 6-chloro-2-((1S,2R)-2-(6-fluoro-2,3-dimethylphenyl)-1-(2H-tetrazol-5-yl)propyl)-3,4-dihydro-2H-benzo[e][1,2]thiazine 1,1-dioxide (10 mg, 0.023 mmol, yield 13%). LC-MS: m/z=448.16, 450.19 [M+H]+; 1x-Cl pattern. 1H NMR (400 MHz, Methanol-d₄) δ 7.67 (d, J=8.4 Hz, 1H), 7.33 (dd, J=8.4, 2.1 Hz, 1H), 7.16 (d, J=2.1 Hz, 1H), 6.86 (dd, J=8.4, 5.7 Hz, 1H), 6.63 (dd, J=12.1, 8.4 Hz, 1H), 6.05 (d, J=11.6 Hz, 1H), 4.14 (t, J=6.8 Hz, 2H), 4.05 (dq, J=13.3, 6.9 Hz, 1H), 3.03 (dt, J=15.4, 7.1 Hz, 1H), 2.69 (dt, J=16.9, 6.4 Hz, 1H), 2.29 (s, 3H), 2.11 (s, 3H), 1.50 (dd, J=6.9, 1.2 Hz, 3H).

### Example 14: synthesis of 5-((1S,2R)-1-(5-chloro-1,1-dioxidobenzo[d]isothiazol-2(3H)-yl)-2-(6-fluoro-2,3-dimethylphenyl) propyl)-1,3,4-oxadiazol-2(3H)-one

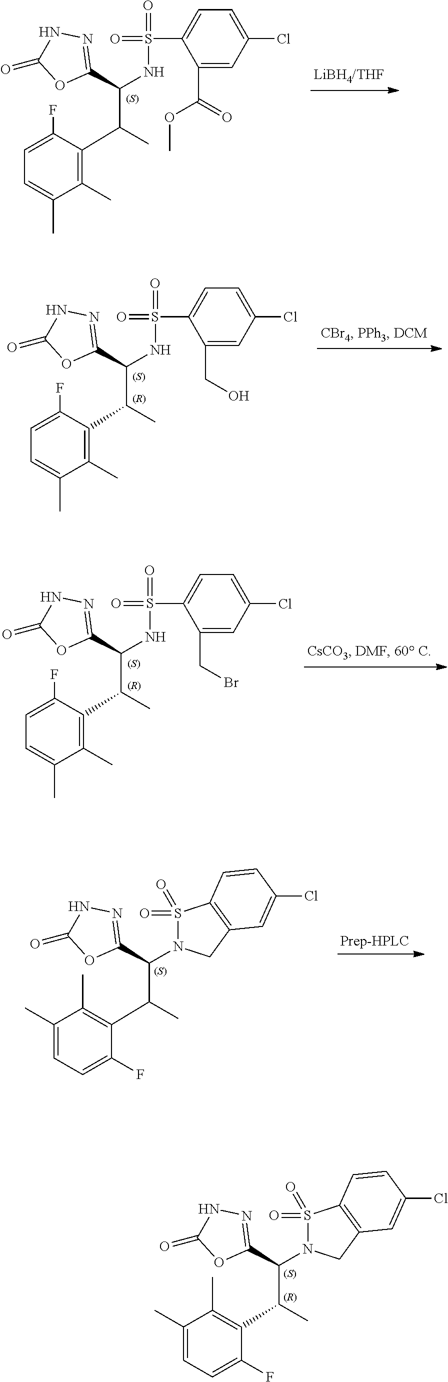

### Step 1: Synthesis of 4-chloro-N-[(1S)-2-(6-fluoro-2,3-dimethylphenyl)-1-(5-oxo-4H-1,3,4-oxadiazol-2-yl) propyl]-2-(hydroxymethyl)benzenesulfonamide

Into a 50 mL round-bottom flask were added methyl 5-chloro-2-(N-((1S)-2-(6-fluoro-2,3-dimethylphenyl)-1-(5-oxo-4,5-dihydro-1,3,4-oxadiazol-2-yl) propyl) sulfamoyl) benzoate (222 mg, 0.45 mmol, 1 equiv) in THF (2 mL). To the above mixture was added LiBH₄ 1M solution in THF (0.54 mL, 0.54 mmol, 1.2 equiv) dropwise at 0° C. The resulting mixture was stirred 1 h at room temperature. The reaction was quenched with water at room temperature. The residue was purified by reverse flash chromatography. This resulted in 4-chloro-N-[(1S)-2-(6-fluoro-2,3-dimethylphenyl)-1-(5-oxo-4H-1,3,4-oxadiazol-2-yl) propyl]-2-(hydroxymethyl) benzenesulfonamide (127 mg, 60.6%).

### Step 2: Synthesis of 2-(bromomethyl)-4-chloro-N-[(1S)-2-(6-fluoro-2,3-dimethylphenyl)-1-(5-oxo-4H-1,3,4-oxadiazol-2-yl) propyl]benzenesulfonamide

To a stirred solution of 4-chloro-N-[(1S)-2-(6-fluoro-2,3-dimethylphenyl)-1-(5-oxo-4H-1,3,4-oxadiazol-2-yl)propyl]-2-(hydroxymethyl)benzenesulfonamide (128 mg, 0.27 mmol, 1 equiv) and carbon tetrabromide (135 mg, 0.41 mmol, 1.5 equiv) in DCM was added triphenylphosphine (143 mg, 0.54 mmol, 2 equiv) in portions at 0° C. The resulting mixture was stirred overnight at 40° C. The resulting mixture was concentrated under vacuum. The residue was purified by Prep-TLC (PE/EtOAc 1:1) to afford 2-(bromomethyl)-4-chloro-N-[(1S)-2-(6-fluoro-2,3-dimethylphenyl)-1-(5-oxo-4H-1,3,4-oxadiazol-2-yl) propyl]benzenesulfonamide (87 mg, 60%).

### Step 3: Synthesis of 5-((1S)-1-(5-chloro-1,1-dioxidobenzo[d]isothiazol-2(3H)-yl)-2-(6-fluoro-2,3-dimethylphenyl) propyl)-1,3,4-oxadiazol-2(3H)-one

To a stirred solution of 2-(bromomethyl)-4-chloro-N-[(1S)-2-(6-fluoro-2,3-dimethylphenyl)-1-(5-oxo-4H-1,3,4-oxadiazol-2-yl) propyl]benzenesulfonamide (87 mg, 0.16 mmol, 1 equiv) in DMF was added Cs₂CO₃ (106.4 mg, 0.33 mmol, 2 equiv) in portions at room temperature. The resulting mixture was stirred 60 min at 60° C. The residue was purified by reverse flash chromatography. This resulted in 5-((1S)-1-(5-chloro-1,1-dioxidobenzo[d]isothiazol-2(3H)-yl)-2-(6-fluoro-2,3-dimethylphenyl) propyl)-1,3,4-oxadiazol-2(3H)-one (50 mg, 67.76%).

### Step 4: Synthesis of 5-((1S,2R)-1-(5-chloro-1,1-dioxidobenzo[d]isothiazol-2(3H)-yl)-2-(6-fluoro-2,3-dimethylphenyl) propyl)-1,3,4-oxadiazol-2(3H)-one

The crude product (50 mg) was purified by Chiral-Prep-HPLC. This resulted in 5-chloro-2-[(1S,2R)-2-(6-fluoro-2,3-dimethylphenyl)-1-(5-oxo-4H-1,3,4-oxadiazol-2-yl)propyl]-3H-llambda6,2-benzothiazole-1,1-dione (25.1 mg, 45.8%). LCMS: (ES, m/z): (M−H)=450.05, 1H NMR (400 MHz, Methanol-d₄) δ 7.91-7.81 (d, J=8.3 Hz, 1H), 7.70-7.66 (m, 1H), 7.65-7.63 (m, 1H), 7.06-6.97 (dd, J=8.4, 5.8 Hz, 1H), 6.84-6.79 (dd, J=12.0, 8.3 Hz, 1H), 5.30-5.27 (dd, J=11.5, 1.5 Hz, 1H), 4.89-4.66 (d, J=14.6 Hz, 2H), 4.00-3.92 (d, J=14.6 Hz, 1H), 2.39-2.36 (s, 3H), 2.33-2.17 (s, 3H), 1.43-1.37 (d, J=6.9 Hz, 3H).

### Example 15: 5-((1R,2S)-1-(5-chloro-7-methoxy-1,1-dioxido-3-oxobenzo[d]isothiazol-2(3H)-yl)-2-(6-fluoro-2,3-dimethylphenyl) propyl)-1,3,4-oxadiazol-2(3H)-one

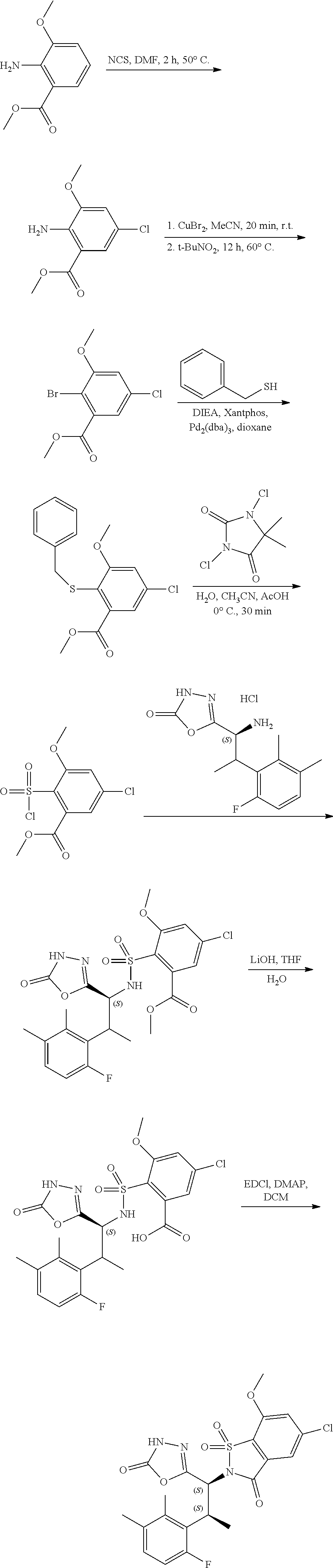

### Step 1: Synthesis of methyl 2-amino-5-chloro-3-methoxybenzoate

Into a 100 mL round-bottom flask were added methyl 2-amino-3-methoxybenzoate (2 g, 11 mmol, 1 equiv) in DMF (20 mL) and NCS (1.62 g, 12.1 mmol, 1.1 equiv) at room temperature. The resulting mixture was stirred for 2 h at 50 degrees C. The resulting mixture was extracted with EtOAc (3×15 mL). The combined organic layers were washed with brine (2×10 mL), dried over anhydrous Na₂SO₄. After filtration, the filtrate was concentrated under reduced pressure. The residue was purified by silica gel column chromatography to afford methyl 2-amino-5-chloro-3-methoxybenzoate (2 g, 84%).

### Step 2: Synthesis of methyl 2-bromo-5-chloro-3-methoxybenzoate

Into a 100 mL round-bottom flask were added methyl 2-amino-5-chloro-3-methoxybenzoate (1 g, 4.6 mmol, 1equiv), CuBr₂ (2.07 g, 9.3 mmol, 2 equiv) and CH₃CN (10 mL) at room temperature. The resulting mixture was stirred 20 min at room temperature. Then t-BuNO₂ (0.86 g, 8.3 mmol, 1.8 equiv) was added. The resulting mixture was stirred 30 min at room temperature, then the resulting mixture was stirred overnight at 60 degrees C. The reaction was quenched by the addition of water (10 mL) at room temperature. The resulting mixture was extracted with EtOAc (3×10 mL). The combined organic layers were washed with brine (2×10 mL), dried over anhydrous Na₂SO₄. The residue was purified by silica gel column chromatography to afford methyl 2-bromo-5-chloro-3-methoxybenzoate (0.8 g, 61.7%).

### Step 3: Synthesis of methyl 2-(benzylsulfanyl)-5-chloro-3-methoxybenzoate

Into a 100 mL round-bottom flask, was placed methyl 2-bromo-5-chloro-3-methoxybenzoate (1.6 g, 5.7 mmol, 1 equiv), benzyl mercaptan (0.85 g, 6.9 mmol, 1.2 equiv), DIEA (2.2 g, 17.2 mmol, 3 equiv), Xantphos (0.66 g, 1.15 mmol, 0.2 equiv), Pd₂(dba)₃ (0.52 g, 0.57 mmol, 0.1 equiv), dioxane (15 mL). The resulting mixture was stirred overnight at 100 degrees C. under nitrogen atmosphere. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography to afford methyl 2-(benzylsulfanyl)-5-chloro-3-methoxybenzoate (1.2 g, 65%).

### Step 4: Synthesis of methyl 5-chloro-2-(chlorosulfonyl)-3-methoxybenzoate

Into a 250 mL round-bottom flask, the mixture of methyl 2-(benzylsulfanyl)-5-chloro-3-methoxybenzoate (1.3 g, 4 mmol, 1 equiv) in MeCN were added 1,3-dichloro-5,5-dimethylimidazolidine-2,4-dione (1.6 g, 8 mmol, 2 equiv), AcOH (0.90 mL, 15.7 mmol, 3.9 equiv) and H₂O (0.70 mL) in portions at 0 degrees C. The resulting mixture was stirred for 30 min at 0 degrees C. The resulting mixture was extracted with EtOAc (3×15 mL). The combined organic layers were washed with brine (2×10 mL), dried over anhydrous Na₂SO₄. After filtration, the filtrate was concentrated under reduced pressure to afford methyl 5-chloro-2-(chlorosulfonyl)-3-methoxybenzoate (1 g, 83.1%).

### Step 5: Synthesis of methyl 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoate

Into a 100 mL round-bottom flask, the mixture of 5-[(1S)-1-amino-2-(6-fluoro-2,3-dimethylphenyl) propyl]-3H-1,3,4-oxadiazol-2-one hydrochloride (200 mg, 0.66 mmol, 1 equiv) in pyridine (5 mL) was added methyl 5-chloro-2-(chlorosulfonyl)-3-methoxybenzoate (400 mg, 1.33 mmol, 2 equiv) dropwise at 0 degrees C. The resulting mixture was stirred overnight at room temperature. The mixture was quenched with water. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography to afford methyl 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoate (100 mg, 28.5%).

### Step 6: Synthesis of 5-chloro-2-(N-((1S)-2-(6-fluoro-2,3-dimethylphenyl)-1-(5-oxo-4,5-dihydro-1,3,4-oxadiazol-2-yl) propyl) sulfamoyl)-3-methoxybenzoic acid

Into a 8 mL round-bottom flask were added methyl 5-chloro-2-{[(1S)-2-(6-fluoro-2,3-dimethylphenyl)-1-(5-oxo-4H-1,3,4-oxadiazol-2-yl)propyl]sulfamoyl}-3-methoxybenzoate (100 mg, 0.19 mmol, 1 equiv) in THF (3 mL), water (1 mL) and LiOH·H₂O (15.9 mg, 0.38 mmol, 2.0 equiv) at room temperature. The resulting mixture was stirred for 2 h at 60 degrees C. The mixture was acidified to pH 5. The residue was purified by reverse flash chromatography to afford 5-chloro-2-{[(1S)-2-(6-fluoro-2,3-dimethylphenyl)-1-(5-oxo-4H-1,3,4-oxadiazol-2-yl)propyl]sulfamoyl}-3-methoxybenzoic acid (70 mg, 71.9%).

### Step 7: Synthesis of 5-((1S,2R)-1-(5-chloro-7-methoxy-1,1-dioxido-3-oxobenzo[d]isothiazol-2(3H)-yl)-2-(6-fluoro-2,3-dimethylphenyl) propyl)-1,3,4-oxadiazol-2(3H)-one

Into a 20 mL round-bottom flask were added 5-chloro-2-{[(1S)-2-(6-fluoro-2,3-dimethylphenyl)-1-(5-oxo-4H-1,3,4-oxadiazol-2-yl)propyl]sulfamoyl}-3-methoxybenzoic acid (120 mg, 0.23 mmol, 1.00 equiv), EDCI (89.5 mg, 0.47 mmol, 2.0 equiv), DMAP (2.85 mg, 0.023 mmol, 0.1 equiv) and DCM (3 mL) at room temperature. The resulting mixture was stirred for 2 h at room temperature. The resulting mixture was extracted with CH₂Cl₂ (3×10 mL). The combined organic layers were washed with brine (2×10 mL), dried over anhydrous MgSO₄. After filtration, the filtrate was concentrated under reduced pressure. The residue was purified by reverse flash chromatography. The crude product was purified by Chiral-Prep-HPLC to afford 5-((1S,2R)-1-(5-chloro-7-methoxy-1,1-dioxido-3-oxobenzo[d]isothiazol-2(3H)-yl)-2-(6-fluoro-2,3-dimethylphenyl)propyl)-1,3,4-oxadiazol-2(3H)-one (9.4 mg, 8.12%). LC-MS (ES, m/z): [M+H]: 496.101H NMR (400 MHz, Methanol-d₄) δ 7.73 (d, J=5.8 Hz, 2H), 7.08 (dd, J=8.4, 5.8 Hz, 1H), 6.83 (dd, J=12.1, 8.4 Hz, 1H), 5.95 (dd, J=11.9, 2.9 Hz, 1H), 4.61 (dq, J=13.4, 7.0 Hz, 1H), 4.12 (s, 3H), 2.40 (s, 3H), 2.28 (s, 3H), 1.38 (d, J=7.0 Hz, 3H).

### Example 16: 5-((1S)-1-(6-chloro-1,1-dioxido-4-oxo-3,4-dihydro-2H-benzo[e][1,2]thiazin-2-yl)-2-(6-fluoro-2,3-dimethylphenyl)propyl)-1,3,4-oxadiazol-2(3H)-one

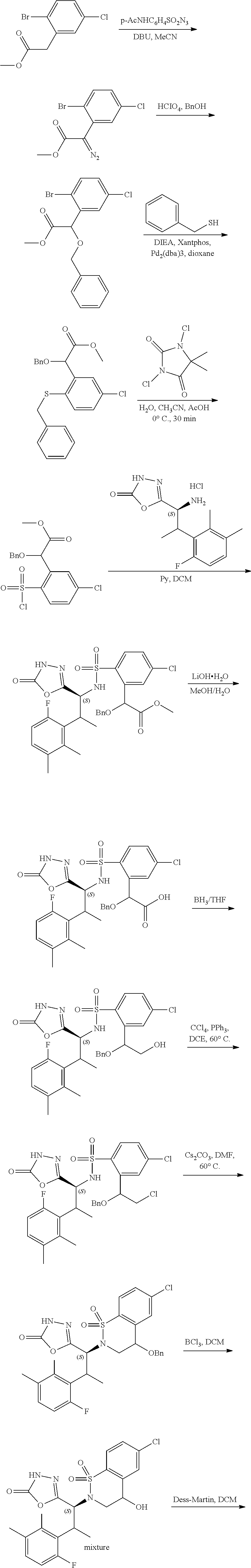

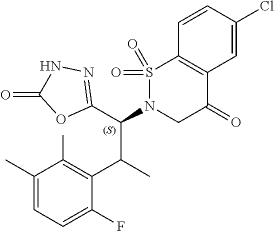

### Step 1: Synthesis of methyl 2-(2-bromo-5-chlorophenyl)-2-diazoacetate

To a stirred solution/mixture of methyl 2-(2-bromo-5-chlorophenyl) acetate (10 g, 37.9 mmol, 1 equiv) in ACN (100 mL) was added 4-acetamidobenzene-1-sulfonyl azide (13.7 g, 56.9 mmol, 1.5 equiv), DBU (9.98 mL, 66.8 mmol, 1.8 equiv) dropwise at 0° C. The resulting mixture was stirred overnight at room temperature. The reaction was quenched by the addition of sat. NH₄Cl (aq.) (100 mL) at room temperature. The resulting mixture was extracted with EtOAc (3×100 mL). The combined organic layers were washed with brine (1×200 mL), dried over anhydrous Na₂SO₄. After filtration, the filtrate was concentrated under reduced pressure. The residue was purified by silica gel column chromatography methyl 2-(2-bromo-5-chlorophenyl)-2-diazoacetate (10 g, 91%).

### Step 2: Synthesis of methyl 2-(benzyloxy)-2-(2-bromo-5-chlorophenyl) acetate methyl 2-(benzyloxy)-2-(2-bromo-5-chlorophenyl)acetate

To a stirred solution of methyl 2-(2-bromo-5-chlorophenyl)-2-diazoacetate (11 g, 38 mmol, 1 equiv) and phenylmethanol (41.1 g, 380 mmol, 10 equiv) was added HClO₄ (700 μL, 12.2 mmol, 0.32 equiv) dropwise at 0° C. The resulting mixture was stirred 3 h at room temperature. The resulting mixture was concentrated under vacuum. The residue was purified by silica gel column chromatography to afford methyl 2-(benzyloxy)-2-(2-bromo-5-chlorophenyl) acetate (9.3 g, 66.2%).

### Step 3: Synthesis of methyl 2-(benzyloxy)-2-[2-(benzylsulfanyl)-5-chlorophenyl]acetate

To a stirred mixture of methyl 2-(benzyloxy)-2-(2-bromo-5-chlorophenyl) acetate (9.3 g, 25.2 mmol, 1 equiv) in dioxane (162 mL) were added Pd₂(dba)₃ (2.3 g, 2.52 mmol, 0.1 equiv), XantPhos (2.54 g, 4.38 mmol, 0.2 equiv) and DIEA (9.76 g, 75.5 mmol, 3 equiv) benzyl mercaptan (3.75 g, 30.2 mmol, 1.2 equiv) at room temperature. The resulting mixture was stirred 3 days at 100° C. under nitrogen atmosphere. The resulting mixture was diluted with EtOAc (200 mL). The resulting mixture was filtered, the filter cake was washed with EtOAc (2×100 mL). The filtrate was concentrated under reduced pressure. The residue was purified by silica gel column chromatography to afford methyl 2-(benzyloxy)-2-[2-(benzylsulfanyl)-5-chlorophenyl]acetate (7 g, 60.6%).

### Step 4: Synthesis afford methyl 2-(benzyloxy)-2-[5-chloro-2-(chlorosulfonyl)phenyl]acetate

To a stirred solution/mixture of methyl 2-(benzyloxy)-2-[2-(benzylsulfanyl)-5-chlorophenyl]acetate (8.83 g, 21.4 mmol, 1 equiv) and H₂O (3.47 mL) in acetonitrile was added AcOH (6 mL) at 0° C. To the above mixture was added 1,3-dichloro-5,5-dimethylimidazolidine-2,4-dione (8.43 g, 42.8 mmol, 2 equiv) in portions at 0° C. The resulting mixture was stirred for additional 30 min at 0° C. The reaction was quenched by the addition of water/ice (25 mL) at 0° C. The resulting mixture was extracted with EtOAc (2×100 mL). The combined organic layers were washed with brine (1×400 mL), dried over anhydrous Na₂SO₄. After filtration, the filtrate was concentrated under reduced pressure. The residue was purified by silica gel column chromatography to afford methyl 2-(benzyloxy)-2-[5-chloro-2-(chlorosulfonyl) phenyl]acetate (8.2 g, 98.5%).

### Step 5: Synthesis of methyl 2-(benzyloxy)-2-(5-chloro-2-(N-((1S)-2-(6-fluoro-2,3-dimethylphenyl)-1-(5-oxo-4,5-dihydro-1,3,4-oxadiazol-2-yl) propyl) sulfamoyl) phenyl) acetate

To a stirred solution of 5-[(1S)-1-amino-2-(6-fluoro-2,3-dimethylphenyl) propyl]-3H-1,3,4-oxadiazol-2-one hydrochloride (5.75 g, 19.1 mmol, 0.9 equiv) in pyridine (25 ml) was added methyl 2-(benzyloxy)-2-[5-chloro-2-(chlorosulfonyl) phenyl]acetate (8.2 g, 21.1 mmol, 1 equiv) in DCM (100 ml) dropwise at 0° C. The resulting mixture was stirred overnight at room temperature. The resulting mixture was diluted with DCM (100 mL). The resulting mixture was washed with 1×250 mL of brine. The resulting mixture was concentrated under vacuum. The residue was purified by silica gel column chromatography to afford methyl 2-(benzyloxy)-2-(5-chloro-2-(N-((1S)-2-(6-fluoro-2,3-dimethylphenyl)-1-(5-oxo-4,5-dihydro-1,3,4-oxadiazol-2-yl) propyl) sulfamoyl) phenyl) acetate (7.1 g, 65.03%).

### Step 6: Synthesis of 2-(benzyloxy)-2-(5-chloro-2-(N-((1S)-2-(6-fluoro-2,3-dimethylphenyl)-1-(5-oxo-4,5-dihydro-1,3,4-oxadiazol-2-yl) propyl) sulfamoyl) phenyl) acetic acid

To a stirred solution/mixture of methyl 2-(benzyloxy)-2-(5-chloro-2-(N-((1S)-2-(6-fluoro-2,3-dimethylphenyl)-1-(5-oxo-4,5-dihydro-1,3,4-oxadiazol-2-yl)propyl)sulfamoyl) phenyl)acetate (6.4 g, 10.4 mmol, 1 equiv) and H₂O (32 mL) in MeOH (32 mL) was added LiOH·H₂O (2172 mg, 51.8 mmol, 5 equiv) in portions at room temperature. The resulting mixture was stirred for 1 h at room temperature. The resulting mixture was diluted with water (20 mL). The mixture was acidified to pH 6 with HCl (2M). The resulting mixture was extracted with EtOAc (3×100 mL). The combined organic layers were washed with brine (1×200 mL), dried over anhydrous Na₂SO₄. After filtration, the filtrate was concentrated under reduced pressure. The crude resulting product was used in the next step directly without further purification.

### Step 7: Synthesis of 2-[1-(benzyloxy)-2-hydroxyethyl]-4-chloro-N-[(1S)-2-(6-fluoro-2,3-dimethylphenyl)-1-(5-oxo-4H-1,3,4-oxadiazol-2-yl) propyl]benzenesulfonamide

To a stirred solution of 2-(benzyloxy)-2-(5-chloro-2-(N-((1S)-2-(6-fluoro-2,3-dimethylphenyl)-1-(5-oxo-4,5-dihydro-1,3,4-oxadiazol-2-yl)propyl)sulfamoyl)phenyl)acetic acid (6.2 g, 10.3 mmol, 1 equiv) in THF (62 mL) was added BH₃-THF 1M (62 mL, 62 mmol, 6 equiv) dropwise at 0° C. The resulting mixture was stirred for 5 h at room temperature. The reaction was quenched by the addition of MeOH (2 mL) at room temperature. The resulting mixture was concentrated under vacuum. The residue was purified by reverse flash chromatography. This resulted in 2-[1-(benzyloxy)-2-hydroxyethyl]-4-chloro-N-[(1S)-2-(6-fluoro-2,3-dimethylphenyl)-1-(5-oxo-4H-1,3,4-oxadiazol-2-yl) propyl]benzenesulfonamide (2.4 g, 39.63%).

### Step 8: Synthesis of 2-[1-(benzyloxy)-2-chloroethyl]-4-chloro-N-[(1S)-2-(6-fluoro-2,3-dimethylphenyl)-1-(5-oxo-4H-1,3,4-oxadiazol-2-yl) propyl]benzene sulfonamide

To a stirred solution of 2-[1-(benzyloxy)-2-hydroxyethyl]-4-chloro-N-[(1S)-2-(6-fluoro-2,3-dimethylphenyl)-1-(5-oxo-4H-1,3,4-oxadiazol-2-yl)propyl]benzenesulfonamide (1.32 g, 0.053 mmol, 1equiv) and PPh₃ (1173 mg, 4.47 mmol, 2 equiv) in DCE was added CCl₄ (619 mg, 4.03 mmol, 1.8 equiv) at 0° C. The resulting mixture was stirred for 15 min at 60° C. The resulting mixture was concentrated under vacuum. The residue was purified by reverse flash chromatography. This resulted in 2-[1-(benzyloxy)-2-chloroethyl]-4-chloro-N-[(1S)-2-(6-fluoro-2,3-dimethylphenyl)-1-(5-oxo-4H-1,3,4-oxadiazol-2-yl) propyl]benzene sulfonamide (615 mg, 45.2%).

### Step 9: Synthesis of 5-((1S)-1-(4-(benzyloxy)-6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

To a stirred mixture of 2-[1-(benzyloxy)-2-chloroethyl]-4-chloro-N-[(1S)-2-(6-fluoro-2,3-dimethylphenyl)-1-(5-oxo-4H-1,3,4-oxadiazol-2-yl) propyl]benzenesulfonamide (325 mg, 0.53 mmol, 1 equiv) in DMF was added Cs₂CO₃ (522 mg, 1.60 mmol, 3 equiv) in portions at room temperature. The resulting mixture was stirred for 60 min at 60° C. The residue was purified by reverse flash chromatography. This resulted in 5-((1S)-1-(4-(benzyloxy)-6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (225 mg, 73.6%).

### Step 10: Synthesis of 5-((1S)-1-(6-chloro-4-hydroxy-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

To a stirred solution/mixture of 5-((1S)-1-(4-(benzyloxy)-6-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (225 mg, 0.39 mmol, 1 equiv) in DCM (1.9 mL) was added boron trichloride (1.57 mL, 1.57 mmol, 4 equiv) dropwise at 0° C. under nitrogen atmosphere. The resulting mixture was stirred for 1 h at room temperature. The reaction was quenched with water at 0° C. The resulting mixture was extracted with EtOAc (3×10 mL). The combined organic layers were washed with brine (1×20 mL), dried over anhydrous Na₂SO₄. The residue was purified by Prep-TLC to afford 5-((1S)-1-(6-chloro-4-hydroxy-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (130 mg, 68.58%).

### Step 11: Synthesis of 5-((1S)-1-(6-chloro-1,1-dioxido-4-oxo-3,4-dihydro-2H-benzo[e][1,2]thiazin-2-yl)-2-(6-fluoro-2,3-dimethylphenyl)propyl)-1,3,4-oxadiazol-2(3H)-one

To a stirred solution/mixture of 5-((1S)-1-(6-chloro-4-hydroxy-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (130 mg, 0.27 mmol, 1 equiv) in DCM was added Dess-Martin (228 mg, 0.54 mmol, 2 equiv) in portions at room temperature. The resulting mixture was stirred 60 min at room temperature. The resulting mixture was filtered, the filter cake was washed with DCM (2×10 mL). The filtrate was concentrated under reduced pressure. The residue was purified by reverse flash chromatography. This resulted in 5-((1S)-1-(6-chloro-4-hydroxy-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (55.8 mg, 42.4%).

LC-MS (ES, m/z): M−H=477.95. 1H NMR (400 MHz, Methanol-d₄) δ 7.87-7.61 (m, 3H), 7.02-6.99 (dd, J=8.4, 5.7 Hz, 1H), 6.77-6.72 (dd, J=12.1, 8.3 Hz, 1H), 5.61-5.50 (dd, J=11.9, 1.9 Hz, 1H), 4.67-4.62 (d, 1H), 3.92-3.86 (m, 1H), 2.34-2.03 (m, 6H), 1.47-1.45 (dd, J=6.9, 1.2 Hz, 2H), 1.29-1.27 (dd, J=21.6, 6.8 Hz, 1H).

### Example 17: 5-((1S)-1-(6-chloro-1,1-dioxido-3-oxo-3,4-dihydro-2H-benzo[e][1,2]thiazin-2-yl)-2-(6-fluoro-2,3-dimethylphenyl) propyl)-1,3,4-oxadiazol-2(3H)-one

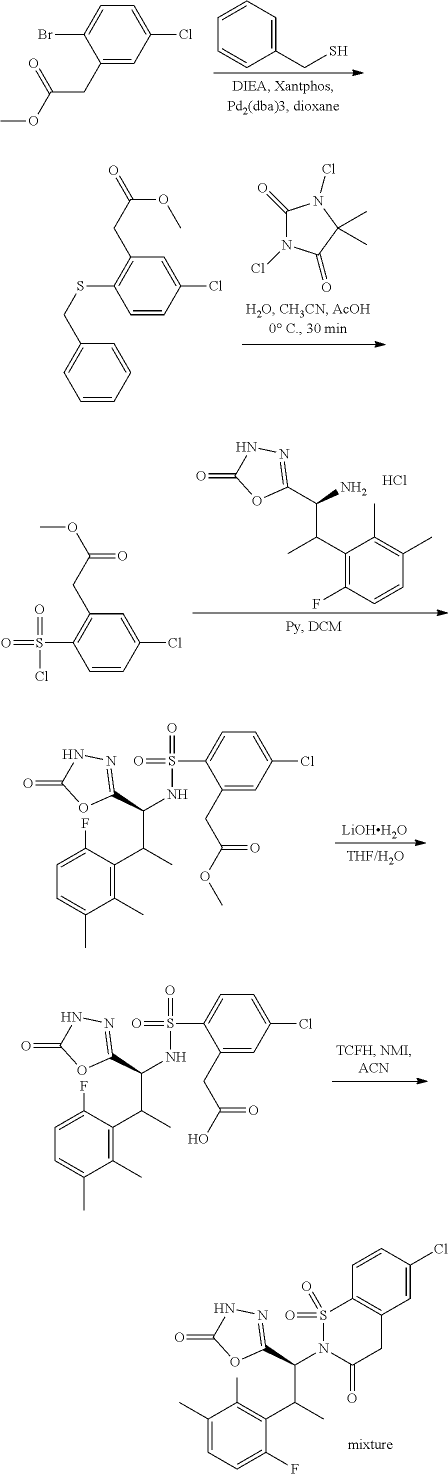

### Step 1: Synthesis of methyl 2-[2-(benzylsulfanyl)-5-chlorophenyl]acetate

Into a 100 mL round-bottom flask were added methyl 2-(2-bromo-5-chlorophenyl) acetate (1 g, 3.80 mmol, 1 equiv), dioxane (10 mL, 118 mmol) and benzyl mercaptan (0.57 g, 4.55 mmol, 1.2equiv), DIEA (1.47 g, 11.4 mmol, 3equiv). To the above mixture was added XantPhos (0.22 g, 0.38 mmol, 0.1 equiv), Pd₂(dba)₃ (0.17 g, 0.19 mmol, 0.05equiv). The resulting mixture was stirred overnight at 100 degrees C. under nitrogen atmosphere. The reaction was quenched with water. The resulting mixture was extracted with EtOAc (3×20 mL). The combined organic layers were washed with brine (1×35 mL), dried over anhydrous Na₂SO₄. After filtration, the filtrate was concentrated under reduced pressure. The resulting mixture was concentrated under vacuum. The residue was purified by silica gel column chromatography to afford methyl 2-[2-(benzylsulfanyl)-5-chlorophenyl]acetate (1 g, 85.9%).

### Step 2: Synthesis of methyl 2-[5-chloro-2-(chlorosulfonyl) phenyl]acetate

Into a 50 mL 3-necked round-bottom flask were added methyl 2-[2-(benzylsulfanyl)-5-chlorophenyl]acetate (400 mg, 1.30 mmol, 1 equiv) and CH₃CN (4 mL). This was followed by the addition of H₂O (244 μL, 13.5 mmol, 10 equiv), AcOH (280 μL, 4. 9 mmol, 3.8 equiv), 1,3-dichloro-5,5-dimethylimidazolidine-2,4-dione (513 mg, 2.6 mmol, 2 equiv) dropwise at 0 degrees C. The resulting mixture was stirred 30 min at 0 degrees C. under nitrogen atmosphere. The reaction was quenched with water. The resulting mixture was extracted with EtOAc. The combined organic layers were washed with brine, dried over anhydrous Na₂SO₄. After filtration, the filtrate was concentrated. This resulted in methyl 2-[5-chloro-2-(chlorosulfonyl) phenyl]acetate (300 mg, 81.3%).

### Step 3: Synthesis of methyl 2-(5-chloro-2-(N-((1S)-2-(6-fluoro-2,3-dimethylphenyl)-1-(5-oxo-4,5-dihydro-1,3,4-oxadiazol-2-yl) propyl) sulfamoyl) phenyl) acetate

Into an 8 mL vial were added 5-[(1S)-1-amino-2-(6-fluoro-2,3-dimethylphenyl) propyl]-3H-1,3,4-oxadiazol-2-one hydrochloride (50 mg, 0.17 mmol, 1 equiv) and Pyridine (2 mL, 25 mmol, 152 equiv). This was followed by the addition of methyl 2-[5-chloro-2-(chlorosulfonyl) phenyl]acetate (141 mg, 0.5 mmol, 3equiv) in DCM dropwise at 0 degrees C. The resulting mixture was stirred overnight at room temperature. The resulting mixture was concentrated under vacuum. The residue was purified by reverse flash chromatography. This resulted in methyl 2-(5-chloro-2-(N-((1S)-2-(6-fluoro-2,3-dimethylphenyl)-1-(5-oxo-4,5-dihydro-1,3,4-oxadiazol-2-yl) propyl) sulfamoyl) phenyl) acetate (80 mg, 94.3%).

### Step 4: Synthesis of 2-(5-chloro-2-(N-((1S)-2-(6-fluoro-2,3-dimethylphenyl)-1-(5-oxo-4,5-dihydro-1,3,4-oxadiazol-2-yl) propyl) sulfamoyl) phenyl) acetic acid

Into a 8 mL vial were added methyl 2-(5-chloro-2-(N-((1S)-2-(6-fluoro-2,3-dimethylphenyl)-1-(5-oxo-4,5-dihydro-1,3,4-oxadiazol-2-yl) propyl) sulfamoyl) phenyl) acetate (20 mg, 0.039 mmol, 1 equiv), THF (1 mL), lithium hydrate (4.92 mg, 0.12 mmol, 3 equiv), H₂O (0.3 mL). The resulting mixture was stirred for additional 2 h at 60 degrees C. The resulting mixture was concentrated under vacuum. The crude product was used in the next step directly without further purification.

### Step 5: Synthesis of 5-((1S)-1-(6-chloro-1,1-dioxido-3-oxo-3,4-dihydro-2H-benzo[e][1,2]thiazin-2-yl)-2-(6-fluoro-2,3-dimethylphenyl)propyl)-1,3,4-oxadiazol-2(3H)-one

Into an 8 mL vial were added 2-(5-chloro-2-(N-((1S)-2-(6-fluoro-2,3-dimethylphenyl)-1-(5-oxo-4,5-dihydro-1,3,4-oxadiazol-2-yl)propyl)sulfamoyl)phenyl)acetic acid (40 mg, 0.080 mmol, 1 equiv), ACN (2 mL), Chloro-N,N,N′,N′-tetramethylformamidinium hexafluorophosphate (27 mg, 0.096 mmol, 1.2equiv) and N-methyl imidazole (23 μL, 0.28 mmol, 3.5equiv). The resulting mixture was stirred overnight at 60 degrees C. The resulting mixture was concentrated under vacuum. The residue was purified by Prep-TLC to afford 5-((1S)-1-(6-chloro-1,1-dioxido-3-oxo-3,4-dihydro-2H-benzo[e][1,2]thiazin-2-yl)-2-(6-fluoro-2,3-dimethylphenyl)propyl)-1,3,4-oxadiazol-2(3H)-one. (0.6 mg, 1.56%). LC-MS (ES, m/z): [M−H]: 479.00. ¹H NMR (300 MHz, Methanol-d₄) δ 7.57 (d, J=8.3 Hz, 1H), 7.47 (dd, J=8.3, 2.0 Hz, 1H), 7.38 (d, J=1.9 Hz, 1H), 6.69 (dd, J=8.3, 5.7 Hz, 1H), 6.38 (dd, J=11.8, 8.4 Hz, 1H), 5.86 (s, 1H), 4.62 (s, 3H), 4.48-4.35 (m, 1H), 2.08 (d, J=15.7 Hz, 6H), 1.52-1.42 (m, 3H), 1.31 (s, 3H).

### Example 18: 5-((1S,2R)-1-(1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

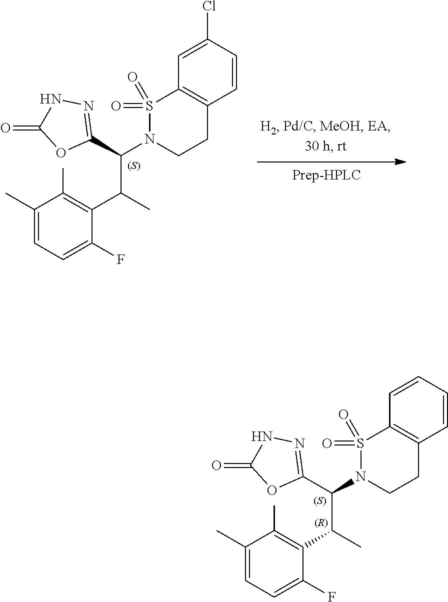

Into a 50 mL round-bottom flask were added 5-((1S)-1-(7-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (120 mg, 0.26 mmol, 1 equiv) and MeOH (5 mL), EtOAc (5 mL) at room temperature. To the above mixture was added Pd/C (10%) (274 mg, 2.6 mmol, 10 equiv) in portions at room temperature. The resulting mixture was stirred overnight at room temperature under hydrogen atmosphere. The resulting mixture was filtered, the filter cake was washed with MeOH (3×10 mL). The filtrate was concentrated under reduced pressure. The crude product (100 mg) was purified by Prep-HPLC to afford 5-((1S,2R)-1-(1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (22.2 mg, 21.56%). LC-MS (ES, m/z):M−H=430.10. ¹H NMR (300 MHz, Methanol-d₄) δ 7.74 (dd, J=7.7, 1.4 Hz, 1H), 7.56-7.30 (m, 2H), 7.26 (d, J=7.6 Hz, 1H), 6.95 (dd, J=8.4, 5.7 Hz, 1H), 6.70 (dd, J=12.1, 8.4 Hz, 1H), 5.57 (dd, J=11.8, 1.2 Hz, 1H), 4.24-3.71 (m, 3H), 3.19-3.01 (m, 1H), 2.89 (dt, J=17.2, 7.0 Hz, 1H), 2.33 (s, 3H), 2.19 (s, 3H), 1.42 (dd, J=7.0, 1.2 Hz, 3H).

### Example 19: 5-((1S,2R)-1-(6-chloro-4-hydroxy-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

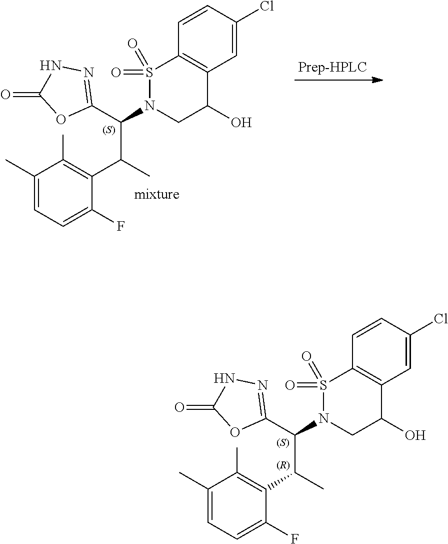

The crude product from example 16 (step 11) (90 mg) was purified by Chiral-Prep-HPLC. This resulted in 5-((1S,2R)-1-(6-chloro-4-hydroxy-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one. (19.7 mg, 21.9%). LC-MS (ES, m/z): M−H=480.08. 1H NMR (400 MHz, Methanol-d₄) δ 7.77-7.31 (m, 3H), 6.99-6.63 (m, 1H), 5.55-5.51 (dd, J=11.9, 1.9 Hz, 1H), 4.87-4.60 (m, 1H), 3.96-3.74 (m, 2H), 3.28-3.25 (m, 1H), 2.33-2.13 (m, 6H), 1.45-1.26 (m. 2H).

### Example 20: 5-((1S)-1-(6-chloro-4-methyl-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

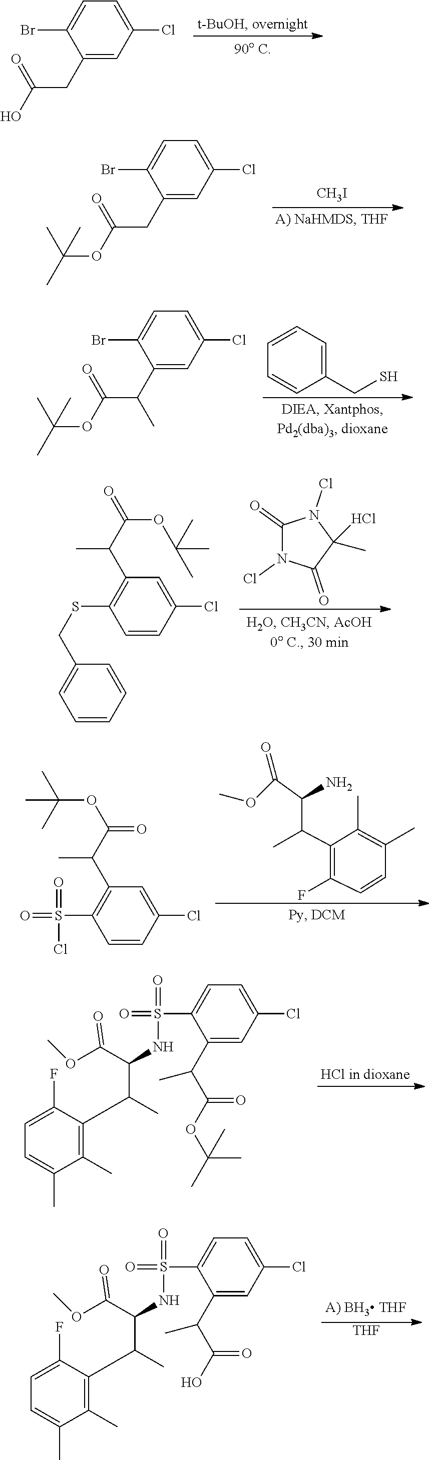

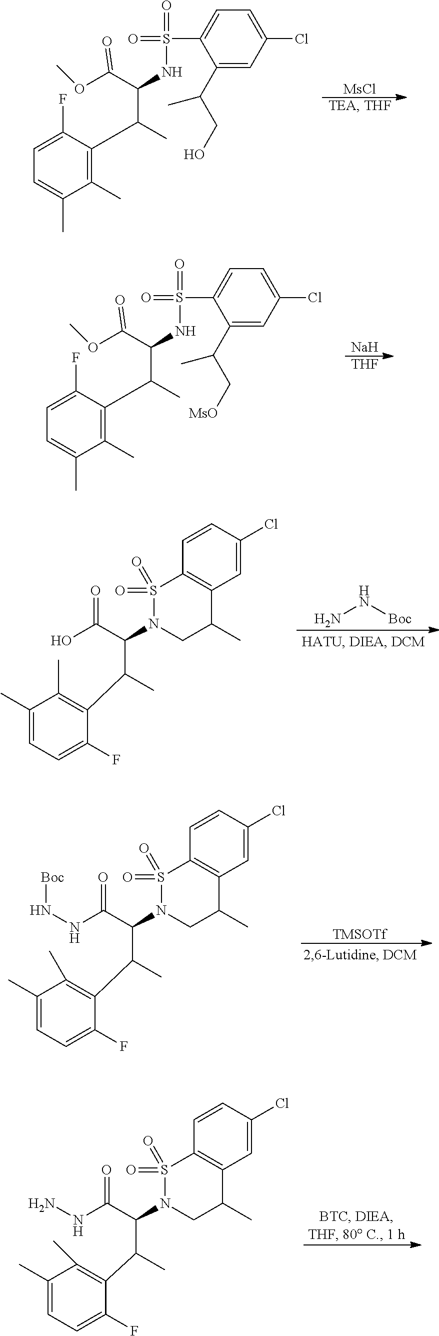

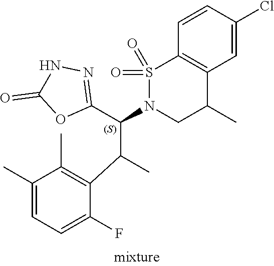

### Step 1: Synthesis of tert-butyl 2-(2-bromo-5-chlorophenyl) propanoate

Into a 250 mL 3-necked round-bottom flask were added tert-butyl 2-(2-bromo-5-chlorophenyl) acetate (4 g, 13 mmol, 1 equiv) and THF (40 mL) at room temperature. To the above mixture was added NaHMDS (3.60 g, 19.6 mmol, 1.5 equiv) dropwise at 0° C. The resulting mixture was stirred for additional 45 min at room temperature. To the above mixture was added methyl iodide (2.79 g, 19.6 mmol, 1.5 equiv) dropwise at 0° C. The resulting mixture was stirred for additional 3 h at room temperature. The reaction was quenched with sat. NH₄Cl (aq.) at 0° C. The resulting mixture was extracted with EtOAc (3×35 mL). The combined organic layers were washed with brine (1×100 mL), dried over anhydrous Na₂SO₄. After filtration, the filtrate was concentrated under reduced pressure. The residue was purified by reverse flash chromatography. This resulted in tert-butyl 2-(2-bromo-5-chlorophenyl) propanoate (3 g, 71.7%).

### Step 2: Synthesis of tert-butyl 2-[2-(benzylsulfanyl)-5-chlorophenyl]propanoate

Into a 40 mL vial were added tert-butyl 2-(2-bromo-5-chlorophenyl) propanoate (2 g, 6.3 mmol, 1 equiv), dioxane (7 mL), DIEA (2426 mg, 18.8 mmol, 3 equiv) and benzyl mercaptan (933 mg, 7.5 mmol, 1.2 equiv), Pd₂(dba)₃ (286 mg, 0.31 mmol, 0.05 equiv), XantPhos (362 mg, 0.63 mmol, 0.10 equiv) at room temperature. The resulting mixture was stirred overnight at 100° C. under nitrogen atmosphere. The reaction was quenched with water at room temperature. The resulting mixture was extracted with EtOAc (3×25 mL). The combined organic layers were washed with brine (1×35 mL), dried over anhydrous Na₂SO₄. After filtration, the filtrate was concentrated under reduced pressure. The residue was purified by silica gel column chromatography to afford tert-butyl 2-[2-(benzylsulfanyl)-5-chlorophenyl]propanoate (1 g, 44%).

### Step 3: Synthesis of tert-butyl 2-[5-chloro-2-(chlorosulfonyl)phenyl]propanoate

Into a 100 mL round-bottom flask were added tert-butyl 2-[2-(benzylsulfanyl)-5-chlorophenyl]propanoate (1.3 g, 3.6 mmol, 1 equiv) and ACN (20 mL) at room temperature. To the above mixture was added AcOH (3 mL), H₂O (2 mL), 1,3-dichloro-5,5-dimethylimidazolidine-2,4-dione (1.4 g, 7.2 mmol, 2 equiv) dropwise at 0° C. The resulting mixture was stirred 30 min at 0° C. The reaction was quenched with water at room temperature. The resulting mixture was extracted with EtOAc (3×15 mL). The combined organic layers were washed with brine (1×25 mL), dried over anhydrous Na₂SO₄. The residue was purified by silica gel column chromatography to afford tert-butyl 2-[5-chloro-2-(chlorosulfonyl) phenyl]propanoate (1.1 g, 90.2%).

### Step 4: Synthesis of methyl (2S)-2-((2-(1-(tert-butoxy)-1-oxopropan-2-yl)-4-chlorophenyl) sulfonamido)-3-(6-fluoro-2,3-dimethylphenyl) butanoate

Into a 40 mL vial were added methyl (2S)-2-amino-3-(6-fluoro-2,3-dimethylphenyl) butanoate (500 mg, 2.1 mmol, 1 equiv) and Pyridine (5 mL, 0.44 mmol) at room temperature. To the above mixture was added tert-butyl 2-[5-chloro-2-(chlorosulfonyl) phenyl]propanoate (1.1 g, 3.2 mmol, 1.6 equiv) in DCM dropwise at 0° C. The resulting mixture was stirred 1 h at room temperature. The resulting mixture was concentrated under vacuum. The residue was purified by reverse flash chromatography. This resulted in methyl (2S)-2-((2-(1-(tert-butoxy)-1-oxopropan-2-yl)-4-chlorophenyl) sulfonamido)-3-(6-fluoro-2,3-dimethylphenyl) butanoate (900 mg, 79.5%).

### Step 5: Synthesis of 2-(5-chloro-2-(N-((2S)-3-(6-fluoro-2,3-dimethylphenyl)-1-methoxy-1-oxobutan-2-yl) sulfamoyl) phenyl) propanoic acid

Into a 100 mL round-bottom flask were added methyl (2S)-2-{2-[1-(tert-butoxy)-1-oxopropan-2-yl]-4-chlorobenzenesulfonamido}-3-(6-fluoro-2,3-dimethylphenyl) butanoate (500 mg, 0.92 mmol, 1 equiv) and HCl (gas) in 1,4-dioxane (5 mL), DCM (2 mL) at room temperature. The resulting mixture was stirred 1 h at room temperature. The resulting mixture was concentrated under vacuum. The residue was purified by reverse flash chromatography. This resulted in 2-(5-chloro-2-(N-((2S)-3-(6-fluoro-2,3-dimethylphenyl)-1-methoxy-1-oxobutan-2-yl) sulfamoyl) phenyl) propanoic acid (300 mg, 67%).

### Step 6: Synthesis of methyl (2S)-2-((4-chloro-2-(1-hydroxypropan-2-yl) phenyl) sulfonamido)-3-(6-fluoro-2,3-dimethylphenyl) butanoate

Into a 20 mL vial were added BH₃-THF (1.23 mL, 1.2 mmol, 2 equiv) and 2-(5-chloro-2-(N-((2S)-3-(6-fluoro-2,3-dimethylphenyl)-1-methoxy-1-oxobutan-2-yl) sulfamoyl) phenyl) propanoic acid (300 mg, 0.62 mmol, 1 equiv) in tetrahydrofuran (3 mL) at room temperature. The resulting mixture was stirred for 20 min at room temperature. The reaction was quenched with MeOH at 0° C. The resulting mixture was concentrated under vacuum. The residue was purified by Prep-TLC to afford methyl (2S)-2-[4-chloro-2-(1-hydroxypropan-2-yl) benzenesulfonamido]-3-(6-fluoro-2,3-dimethylphenyl) butanoate (40 mg, 41.2%).

### Step 7: Synthesis of methyl (2S)-2-((4-chloro-2-(1-((methylsulfonyl)oxy) propan-2-yl) phenyl) sulfonamido)-3-(6-fluoro-2,3-dimethylphenyl) butanoate

Into a 50 mL round-bottom flask were added methyl (2S)-2-[4-chloro-2-(1-hydroxypropan-2-yl) benzenesulfonamido]-3-(6-fluoro-2,3-dimethylphenyl) butanoate (120 mg, 0.25 mmol, 1 equiv) and DCM (5 mL), TEA (212 μL, 1.5 mmol, 6 equiv) at room temperature. To the above mixture was added 2M methanesulfonyl chloride in DCM (254 μL, 0.51 mmol, 2 equiv) dropwise at 0° C. The resulting mixture was stirred 1 h at room temperature. The reaction was quenched with water at room temperature. The resulting mixture was extracted with DCM (3×15 mL). The combined organic layers were washed with brine (1×50 mL), dried over anhydrous Na₂SO₄. After filtration, the filtrate was concentrated under reduced pressure. The residue was purified by Prep-TLC to afford methyl (2S)-2-{4-chloro-2-[1-(methanesulfonyloxy) propan-2-yl]benzenesulfonamido}-3-(6-fluoro-2,3-dimethylphenyl) butanoate (40 mg, 85.8%).

### Step 8: Synthesis of (2S)-2-(6-chloro-4-methyl-1,1-dioxido-3,4-dihydro-2H-benzo[e][1,2]thiazin-2-yl)-3-(6-fluoro-2,3-dimethylphenyl)butanoic acid

Into a 50 mL 3-necked round-bottom flask were methyl (2S)-2-{4-chloro-2-[1-(methanesulfonyloxy) propan-2-yl]benzenesulfonamido}-3-(6-fluoro-2,3-dimethylphenyl) butanoate (100 mg, 0.18 mmol, 1 equiv) and tetrahydrofuran (10 mL) at room temperature. To the above mixture was added sodium hydride (60%) (65.4 mg, 2.73 mmol, 15 equiv) dropwise at 0° C. The resulting mixture was stirred overnight at room temperature. The mixture was acidified to pH 6 with 1M HCl. The resulting mixture was concentrated under vacuum. The residue was purified by reverse flash chromatography. This resulted in (2S)-2-(6-chloro-4-methyl-1,1-dioxido-3,4-dihydro-2H-benzo[e][1,2]thiazin-2-yl)-3-(6-fluoro-2,3-dimethylphenyl) butanoic acid (60 mg, 75.0%).

### Step 9: Synthesis of tert-butyl 2-((2S)-2-(6-chloro-4-methyl-1,1-dioxido-3,4-dihydro-2H-benzo[e][1,2]thiazin-2-yl)-3-(6-fluoro-2,3-dimethylphenyl)butanoyl)hydrazine-1-carboxylate

Into a 50 mL round-bottom flask were added (2S)-2-(6-chloro-4-methyl-1,1-dioxido-3,4-dihydro-2H-benzo[e][1,2]thiazin-2-yl)-3-(6-fluoro-2,3-dimethylphenyl)butanoic acid (20 mg, 0.045 mmol, 1 equiv), DCM (3 mL), tert-butoxycarbohydrazide (7.81 mg, 0.06 mmol, 1.3 equiv), DIEA (17.6 mg, 0.14 mmol, 3 equiv) and HATU (25.9 mg, 0.07 mmol, 1.5 equiv) at room temperature. The resulting mixture was stirred 1 h at room temperature. The resulting mixture was extracted with DCM (3×25 mL). The combined organic layers were washed with brine (1×35 mL), dried over anhydrous Na₂SO₄. After filtration, the filtrate was concentrated under reduced pressure. The residue was purified by Prep-TLC to afford tert-butyl 2-((2S)-2-(6-chloro-4-methyl-1,1-dioxido-3,4-dihydro-2H-benzo[e][1,2]thiazin-2-yl)-3-(6-fluoro-2,3-dimethylphenyl)butanoyl)hydrazine-1-carboxylate (20 mg, 79.40%).

### Step 10: Synthesis of (2S)-2-(6-chloro-4-methyl-1,1-dioxido-3,4-dihydro-2H-benzo[e][1,2]thiazin-2-yl)-3-(6-fluoro-2,3-dimethylphenyl)butanehydrazide

Into a 20 mL vial were added tert-butyl 2-((2S)-2-(6-chloro-4-methyl-1,1-dioxido-3,4-dihydro-2H-benzo[e][1,2]thiazin-2-yl)-3-(6-fluoro-2,3-dimethylphenyl)butanoyl)hydrazine-1-carboxylate (20 mg, 0.04 mmol, 1 equiv) and DCM (2 mL), 2,6-lutidine (77.4 mg, 0.72 mmol, 20 equiv) at room temperature. To the above mixture was added trimethylsilyl triflate (128 mg, 0.58 mmol, 16 equiv) dropwise at 0° C. The resulting mixture was stirred 1 h at room temperature. The resulting mixture was concentrated under vacuum. The residue was purified by reverse flash chromatography. This resulted in (2S)-2-(6-chloro-4-methyl-1,1-dioxido-3,4-dihydro-2H-benzo[e][1,2]thiazin-2-yl)-3-(6-fluoro-2,3-dimethylphenyl) butanehydrazide (10 mg, 61.03%)

### Step 11: Synthesis of 5-((1S)-1-(6-chloro-4-methyl-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

Into an 8 mL vial were added (2S)-2-(6-chloro-4-methyl-1,1-dioxido-3,4-dihydro-2H-benzo[e][1,2]thiazin-2-yl)-3-(6-fluoro-2,3-dimethylphenyl)butanehydrazide (10 mg, 0.022 mmol, 1 equiv) in THF (1 mL), DIEA (7.1 mg, 0.055 mmol, 2.5 equiv) and ditrichloromethyl carbonate (3.3 mg, 0.011 mmol, 0.5 equiv) at room temperature. The resulting mixture was stirred 1 h at 80° C. The resulting mixture was concentrated under vacuum. The crude product was purified by Chiral-Prep-HPLC. This resulted in 6-chloro-2-[(1S)-2-(6-fluoro-2,3-dimethylphenyl)-1-(5-oxo-4H-1,3,4-oxadiazol-2-yl) propyl]-4-methyl-3,4-dihydro-llambda6,2-benzothiazine-1,1-dione (4.6 mg, 43.4%). LC-MS m/z: 478 (M−1). ¹H NMR (300 MHz, DMSO-d6) δ 11.98 (s, 1H), 7.77 (dd, J=8.4, 3.6 Hz, 1H), 7.61-7.48 (m, 2H), 7.03 (dd, J=8.6, 6.0 Hz, 1H), 6.84 (ddd, J=12.5, 8.4, 4.3 Hz, 1H), 5.41 (dd, J=11.7, 2.0 Hz, 1H), 4.08-3.94 (m, 1H), 3.81 (q, J=8.7, 8.2 Hz, 1H), 3.71-3.55 (m, 1H), 3.38 (s, 1H), 2.27 (d, J=8.6 Hz, 3H), 2.17 (d, J=2.2 Hz, 3H), 1.30 (td, J=13.3, 12.5, 6.9 Hz, 6H).

### Example 21: 5-((1S)-1-(6-chloro-4,4-dimethyl-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

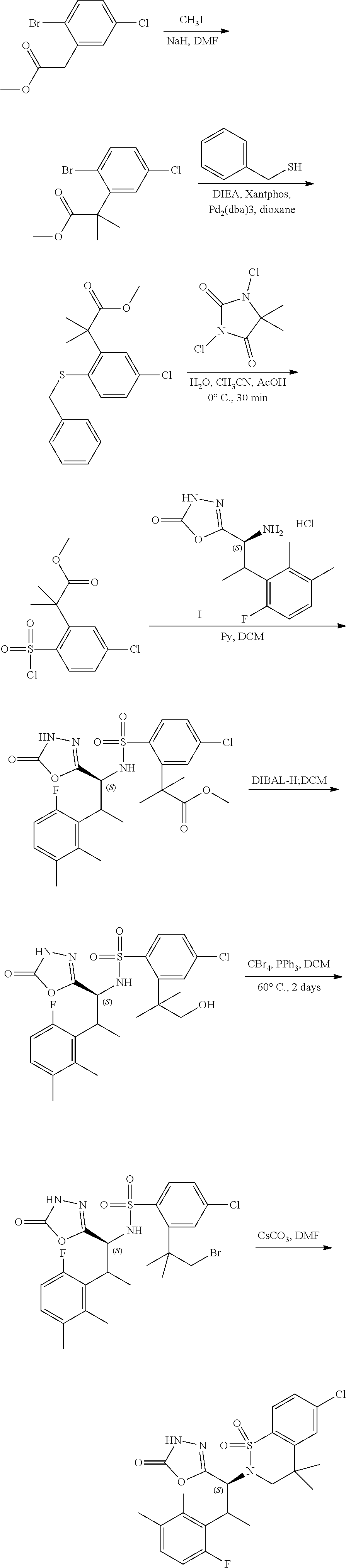

### Step 1: Synthesis of methyl 2-(2-bromo-5-chlorophenyl)-2-methylpropanoate

To a solution of methyl 2-(2-bromo-5-chlorophenyl) acetate (5 g, 18.9 mmol, 1 equiv) in DMF was added sodium hydride (60% in oil, 3 g) at 0 degrees C. The mixture was stirred for 1 h. CH₃I (3.5 mL, 57 mmol, 3 equiv) was added and the mixture was allowed to warm to RT and stirred overnight. The reaction was quenched with sat. NH₄Cl (aq.) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography to afford methyl 2-(2-bromo-5-chlorophenyl)-2-methylpropanoate (4.2 g, 75.9%).

### Step 2: Synthesis of methyl 2-[2-(benzylsulfanyl)-5-chlorophenyl]-2-methylpropanoate

In a 50 mL round-bottom flask were added methyl 2-(2-bromo-5-chlorophenyl)-2-methylpropanoate (2.54 g, 8.7 mmol, 1 equiv) and dioxane (25 mL) at room temperature. To the above mixture was added DIEA (4.6 mL, 26.3 mmol, 3 equiv) Xantphos (1.01 g, 1.74 mmol, 0.2 equiv), Pd₂(dba)₃ (0.80 g, 0.87 mmol, 0.1 equiv), benzyl mercaptan (1.23 mL, 10.4 mmol, 1.2 equiv) dropwise at room temperature. The resulting mixture was stirred for additional overnight at 110° C. under nitrogen atmosphere. The resulting mixture was extracted with EtOAc (3×50 mL). The combined organic layers were washed with brine (1×200 mL), dried over anhydrous Na₂SO₄. After filtration, the filtrate was concentrated under reduced pressure. The residue was purified by silica gel column chromatography to afford methyl 2-[2-(benzylsulfanyl)-5-chlorophenyl]-2-methylpropanoate (900 mg, 30.85%).

### Step 3: Synthesis of methyl 2-[5-chloro-2-(chlorosulfonyl) phenyl]-2-methylpropanoate

To a stirred solution of methyl 2-[2-(benzylsulfanyl)-5-chlorophenyl]-2-methylpropanoate (900 mg, 2.7 mmol, 1 equiv) in CH₃CN (10 mL) were added AcOH (1.5 mL), H₂O (1 mL) and 1,3-dichloro-5,5-dimethylimidazolidine-2,4-dione (1060 mg, 5.4 mmol, 2.0 equiv) in portions at 0° C. The resulting mixture was stirred for 30 min at 0° C. The resulting mixture was extracted with EtOAc (2×10 mL). The combined organic layers were washed with brine (3×50 mL), dried over anhydrous Na₂SO₄. After filtration, the filtrate was concentrated under reduced pressure. The residue was purified by silica gel column chromatography to afford methyl 2-[5-chloro-2-(chlorosulfonyl) phenyl]-2-methylpropanoate (620 mg, 74.1%).

### Step 4: Synthesis of methyl 2-(5-chloro-2-(N-((1S)-2-(6-fluoro-2,3-dimethylphenyl)-1-(5-oxo-4,5-dihydro-1,3,4-oxadiazol-2-yl) propyl)sulfamoyl)phenyl)-2-methylpropanoate

To a stirred solution of 5-[(1S)-1-amino-2-(6-fluoro-2,3-dimethylphenyl)propyl]-3H-1,3,4-oxadiazol-2-one hydrochloride (620 mg, 2.06 mmol, 1 equiv) in DCM (5 mL, 78.7 mmol) were added pyridine (500 μL, 6.2 mmol, 3 equiv) and methyl 2-[5-chloro-2-(chlorosulfonyl)phenyl]-2-methylpropanoate (767 mg, 2.47 mmol, 1.2 equiv) in portions at 0° C. The resulting mixture was stirred for overnight at room temperature. The residue was purified by silica gel column chromatography to afford methyl 2-(5-chloro-2-(N-((1S)-2-(6-fluoro-2,3-dimethylphenyl)-1-(5-oxo-4,5-dihydro-1,3,4-oxadiazol-2-yl) propyl)sulfamoyl)phenyl)-2-methylpropanoate (764 mg, 69%).

### Step 5: Synthesis of 4-chloro-N-[(1S)-2-(6-fluoro-2,3-dimethylphenyl)-1-(5-oxo-4H-1,3,4-oxadiazol-2-yl) propyl]-2-(1-hydroxy-2-methylpropan-2-yl) benzenesulfonamide

To a stirred solution of methyl 2-(5-chloro-2-(N-((1S)-2-(6-fluoro-2,3-dimethylphenyl)-1-(5-oxo-4,5-dihydro-1,3,4-oxadiazol-2-yl)propyl)sulfamoyl)phenyl)-2-methylpropanoate (210 mg, 0.39 mmol, 1 equiv) in DCM was added DIBAL-H (780 μL, 1.2 mmol, 3 equiv) dropwise at 0° C. under nitrogen atmosphere. The resulting mixture was stirred 30 min at room temperature under nitrogen atmosphere. To the above mixture was added DIBAl-H (390 μL, 0.58 mmol, 1.5 equiv) dropwise at 0° C. The resulting mixture was stirred for additional 30 min at room temperature. The reaction was quenched with water at 0° C. The resulting mixture was concentrated under vacuum. The residue was purified by reverse flash chromatography. This resulted in 4-chloro-N-[(1S)-2-(6-fluoro-2,3-dimethylphenyl)-1-(5-oxo-4H-1,3,4-oxadiazol-2-yl) propyl]-2-(1-hydroxy-2-methylpropan-2-yl) benzenesulfonamide (70 mg, 35.2%).

### Step 6: Synthesis of -(1-bromo-2-methylpropan-2-yl)-4-chloro-N-[(1S)-2-(6-fluoro-2,3-dimethylphenyl)-1-(5-oxo-4H-1,3,4-oxadiazol-2-yl) propyl]benzenesulfonamide

To a stirred solution of 4-chloro-N-[(1S)-2-(6-fluoro-2,3-dimethylphenyl)-1-(5-oxo-4H-1,3,4-oxadiazol-2-yl)propyl]-2-(1-hydroxy-2-methylpropan-2-yl)benzenesulfonamide (50 mg, 0.098 mmol, 1 equiv) and PPh₃ (51 mg, 0.2 mmol, 2 equiv) in DCM was added CBr₄ (49 mg, 0.15 mmol, 1.5 equiv) in portions at room temperature. The resulting mixture was stirred for 2 days at 40° C. The residue was purified by Prep-TLC (PE/EtOAc 1:1) to afford 2-(1-bromo-2-methylpropan-2-yl)-4-chloro-N-[(1S)-2-(6-fluoro-2,3-dimethylphenyl)-1-(5-oxo-4H-1,3,4-oxadiazol-2-yl) propyl]benzenesulfonamide (35 mg, 62.3%).

### Step 7: Synthesis of 5-((1S)-1-(6-chloro-4,4-dimethyl-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

In a 25 mL round-bottom flask were added 2-(1-bromo-2-methylpropan-2-yl)-4-chloro-N-[(1S)-2-(6-fluoro-2,3-dimethylphenyl)-1-(5-oxo-4H-1,3,4-oxadiazol-2-yl) propyl]benzenesulfonamide (30 mg, 0.052 mmol, 1 equiv) and DMF (500 μL) at room temperature. To the above mixture was added Cs₂CO₃ (34 mg, 0.10 mmol, 2 equiv) at room temperature. The resulting mixture was stirred 1 h at 60° C. The crude product (25 mg) was purified by Prep-HPLC to afford 5-((1S)-1-(6-chloro-4,4-dimethyl-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (4.0 mg, 15.5%). LC-MS: (ES, m z): [M−H]+=492.05. 1H NMR (400 MHz, Methanol-d₄) δ 7.81 (d, J=8.5 Hz, 1H), 7.62 (d, J=2.0 Hz, 1H), 7.49 (dd, J=8.5, 2.1 Hz, 1H), 7.04-7.02 (dd, J=8.4, 5.8 Hz, 1H), 6.80-6.75 (dd, J=12.0, 8.4 Hz, 1H), 5.68-5.65 (m, 1H), 3.92-3.64 (m, 3H), 2.37 (s, 3H), 2.24 (s, 3H), 1.50-1.45 (m, 3H), 1.29 (s, 3H).

### Example 22: 5-((1S,2R)-1-(6-chloro-4,4-difluoro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

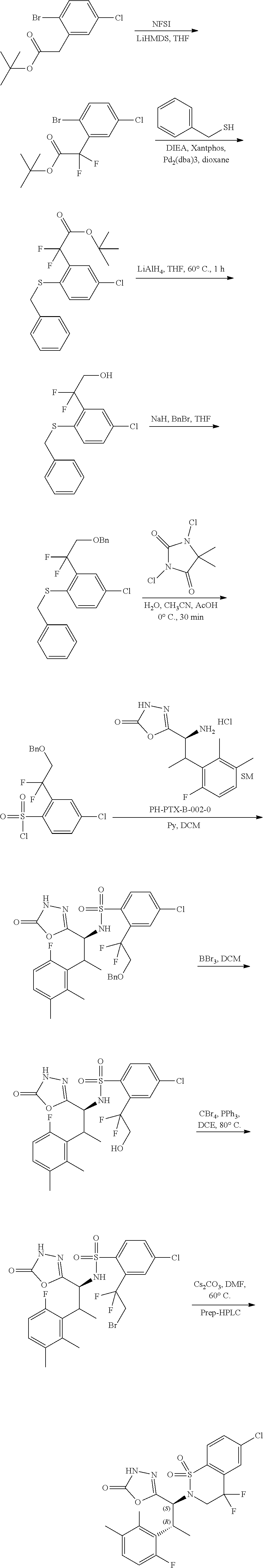

### Step 1: Synthesis of tert-butyl 2-(2-bromo-5-chlorophenyl)-2,2-difluoroacetate

In a 250-mL round bottom flask, to a solution of tert-butyl 2-(2-bromo-5-chlorophenyl) acetate (10 g, 32.7 mmol, 1 equiv) in THF (100 mL) was added dropwise LiHMDS (1 M in THF, 163 mL,) at −78 degrees C. under N₂ atmosphere. The reaction mixture was stirred at −78 degrees C. for 10 mins. Then a solution of N-Fluorobenzenesulfonimide (31 g, 98.2 mmol, 3 equiv) in 3 mL THF was added dropwise and the mixture was stirred 10 h. The reaction was quenched with water/sat. NH₄Cl (10 mL), and then the mixture was extracted with EtOAc (2×150 mL). The combined organic extracts were washed with brine (300 mL), dried over anhydrous Na₂SO₄, and concentrated under vacuum to yield a crude product which was directly purified by flash chromatography (PE). This resulted in tert-butyl 2-(2-bromo-5-chlorophenyl)-2,2-difluoroacetate (7 g, 62.6%).

### Step 2: Synthesis of tert-butyl 2-[2-(benzylsulfanyl)-5-chlorophenyl]-2,2-difluoroacetate

Into a 250 mL round-bottom flask were added tert-butyl 2-(2-bromo-5-chlorophenyl)-2,2-difluoroacetate (7 g, 20.5 mmol, 1 equiv), benzyl mercaptan (3050 mg, 24.6 mmol, 1.2 equiv), DIEA (7950 mg, 61.5 mmol, 3 equiv), Xantphos (2372 mg, 4.1 mmol, 0.2 equiv) and Pd₂(dba)₃ (1877 mg, 2.1 mmol, 0.1 equiv) in dioxane (112 mL) at room temperature. The resulting mixture was stirred 2 h at 110° C. under nitrogen atmosphere. The reaction was quenched with water at room temperature. The resulting mixture was extracted with EtOAc (3×30 mL). The combined organic layers were washed with brine (3×10 mL), dried over anhydrous Na₂SO₄. After filtration, the filtrate was concentrated under reduced pressure. The residue was purified by silica gel column chromatography to afford tert-butyl 2-[2-(benzylsulfanyl)-5-chlorophenyl]-2,2-difluoroacetate (4.9 g, 59.0%).

### Step 3: Synthesis of 2-[2-(benzylsulfanyl)-5-chlorophenyl]-2,2-difluoroethanol

Into a 100 mL 3-necked round-bottom flask were added a solution of tert-butyl 2-[2-(benzylsulfanyl)-5-chlorophenyl]-2,2-difluoroacetate (3800 mg, 9.9 mmol, 1 equiv) in THF, followed by the addition of LiAlH₄ (2 mol/L in THF) (10 mL, 20 mmol, 2.0 equiv,) dropwise at room temperature. The resulting mixture was stirred 30 min at 60° C. under air atmosphere. The reaction was quenched with water at room temperature. The resulting mixture was extracted with DCM. The combined organic layers were washed with brine, dried over anhydrous Na₂SO₄. After filtration, the filtrate was concentrated under reduced pressure. The residue was purified by reverse flash chromatography to afford 2-[2-(benzylsulfanyl)-5-chlorophenyl]-2,2-difluoroethanol (1033 mg, 33.2%).

### Step 4: Synthesis of 2-[2-(benzyloxy)-1,1-difluoroethyl]-1-(benzylsulfanyl)-4-chlorobenzene

Into a 50 mL round-bottom flask were added a solution of 2-[2-(benzylsulfanyl)-5-chlorophenyl]-2,2-difluoroethanol (1033 mg, 3.3 mmol, 1 equiv) in THF and was treated with NaH 60% (118 mg, 4.9 mmol, 1.5 equiv) for 30 min at 0° C. under nitrogen atmosphere followed by the addition of (bromomethyl)benzene (674 mg, 3.94 mmol, 1.2 equiv) dropwise at 0° C. The resulting mixture was stirred overnight at room temperature under air atmosphere. The residue was purified by silica gel column chromatography to afford 2-[2-(benzyloxy)-1,1-difluoroethyl]-1-(benzylsulfanyl)-4-chlorobenzene (1069 mg, 80.5%).

### Step 5: Synthesis of 2-[2-(benzyloxy)-1,1-difluoroethyl]-4-chlorobenzenesulfonyl chloride

Into a 50 mL round-bottom flask were added a solution of 2-[2-(benzyloxy)-1,1-difluoroethyl]-1-(benzylsulfanyl)-4-chlorobenzene (1069 mg, 2.6 mmol, 1 equiv) in MeCN. To the mixture was added H₂O (590 μL) and AcOH (855 μL) at 0° C. followed by 1,3-dichloro-5,5-dimethylimidazolidine-2,4-dione (1040 mg, 5.3 mmol, 2.0 equiv) in portions at 0° C. The resulting mixture was stirred 30 min at 0° C. under air atmosphere. The residue was purified by silica gel column chromatography to afford 2-[2-(benzyloxy)-1,1-difluoroethyl]-4-chlorobenzenesulfonyl chloride (800 mg, 79.5%).

### Step 6: Synthesis of 2-[2-(benzyloxy)-1,1-difluoroethyl]-4-chloro-N-[(1S)-2-(6-fluoro-2,3-dimethylphenyl)-1-(5-oxo-4H-1,3,4-oxadiazol-2-yl) propyl]benzenesulfonamide

To a stirred solution of 5-[(1S)-1-amino-2-(6-fluoro-2,3-dimethylphenyl) propyl]-3H-1,3,4-oxadiazol-2-one hydrochloride (237 mg, 0.79 mmol, 1 equiv) and pyridine (3 mL) was added 2-[2-(benzyloxy)-1,1-difluoroethyl]-4-chlorobenzenesulfonyl chloride (300 mg, 0.79 mmol, 1 equiv) in DCM dropwise at 0° C. The resulting mixture was extracted with EtOAc (2×10 mL). The combined organic layers were washed with brine (1×20 mL), dried over anhydrous Na₂SO₄. After filtration, the filtrate was concentrated under reduced pressure. The residue was purified by silica gel column chromatography to afford 2-[2-(benzyloxy)-1,1-difluoroethyl]-4-chloro-N-[(1S)-2-(6-fluoro-2,3-dimethylphenyl)-1-(5-oxo-4H-1,3,4-oxadiazol-2-yl) propyl]benzenesulfonamide (300 mg, 62.5%).

### Step 7: Synthesis of 4-chloro-2-(1,1-difluoro-2-hydroxyethyl)-N-[(1S)-2-(6-fluoro-2,3-dimethylphenyl)-1-(5-oxo-4H-1,3,4-oxadiazol-2-yl) propyl]benzenesulfonamide

Into a 25 mL round-bottom flask were added 2-[2-(benzyloxy)-1,1-difluoroethyl]-4-chloro-N-[(1S)-2-(6-fluoro-2,3-dimethylphenyl)-1-(5-oxo-4H-1,3,4-oxadiazol-2-yl)propyl]benzenesulfonamide (587 mg, 0.96 mmol, 1 equiv) in DCM (2 mL) followed by the addition of BBr₃ (2900 μL, 2.9 mmol, 3.0 equiv) dropwise at 0° C. The resulting mixture was stirred 30 min at 0° C. under air atmosphere. Then the resulting mixture was stirred 2 h at room temperature under air atmosphere. The reaction was quenched with water at 0° C. The resulting mixture was extracted with DCM (2×5 mL). The combined organic layers were dried over anhydrous MgSO₄. After filtration, the filtrate was concentrated under reduced pressure. The residue was purified by silica gel column chromatography to afford 4-chloro-2-(1,1-difluoro-2-hydroxyethyl)-N-[(1S)-2-(6-fluoro-2,3-dimethylphenyl)-1-(5-oxo-4H-1,3,4-oxadiazol-2-yl) propyl]benzenesulfonamide (300 mg, 60%).

### Step 8: Synthesis of 2-(2-bromo-1,1-difluoroethyl)-4-chloro-N-[(1S)-2-(6-fluoro-2,3-dimethylphenyl)-1-(5-oxo-4H-1,3,4-oxadiazol-2-yl) propyl]benzenesulfonamide

In a 25 mL round-bottom flask were added a solution of 4-chloro-2-(1,1-difluoro-2-hydroxyethyl)-N-[(1S)-2-(6-fluoro-2,3-dimethylphenyl)-1-(5-oxo-4H-1,3,4-oxadiazol-2-yl)propyl]benzenesulfonamide (232 mg, 0.45 mmol, 1 equiv) in DCE (3 mL) and CBr₄ (222 mg, 0.67 mmol, 1.5 equiv). To the mixture was added PPh₃ (234 mg, 0.89 mmol, 2 equiv) in portions at room temperature. The resulting mixture was stirred overnight at 80° C. under air atmosphere. The residue was purified by Prep-TLC to afford 2-(2-bromo-1,1-difluoroethyl)-4-chloro-N-[(1S)-2-(6-fluoro-2,3-dimethylphenyl)-1-(5-oxo-4H-1,3,4-oxadiazol-2-yl) propyl]benzene sulfonamide (184 mg, 70.8%).

### Step 9: Synthesis of 5-((1S,2R)-1-(6-chloro-4,4-difluoro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

To a stirred solution of 2-(2-bromo-1,1-difluoroethyl)-4-chloro-N-[(1S)-2-(6-fluoro-2,3-dimethylphenyl)-1-(5-oxo-4H-1,3,4-oxadiazol-2-yl) propyl]benzenesulfonamide (185 mg, 0.32 mmol, 1 equiv) in DMF (2 mL, 1.29 mmol) was added Cs₂CO₃ (207 mg, 0.63 mmol, 2 equiv) in portions at room temperature. The resulting mixture was stirred 1 h at 60° C. The residue was purified by reverse flash chromatography. This resulted 5-((1S)-1-(6-chloro-4,4-difluoro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (75 mg, 47.1%).

The product (75 mg) was further purified by Prep-HPLC to afford 6-chloro-4,4-difluoro-2-[(1S,2R)-2-(6-fluoro-2,3-dimethylphenyl)-1-(5-oxo-4H-1,3,4-oxadiazol-2-yl)propyl]-3H-llambda6,2-benzothiazine-1,1-dione (17.7 mg, 23.4%). LC-MS (ES, m z): [M/2+H]+=251.90. 1H NMR (300 MHz, DMSO-d₆) δ 12.28 (s, 1H), 8.05-8.00 (m, 2H), 7.95-7.92 (dd, J=8.5, 2.1 Hz, 1H), 7.08-7.03 (dd, J=8.4, 5.8 Hz, 1H), 6.91-6.84 (dd, J=12.3, 8.4 Hz, 1H), 5.65-5.60 (m, 1H), 4.62-4.54 (m, 2H), 3.95-3.88 (tt, J=13.5, 6.3 Hz, 1H), 2.32 (s, 3H), 2.19 (s, 3H), 1.33-1.24 (m, 3H).

### Example 23: 5-((1S,2R)-1-(5-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

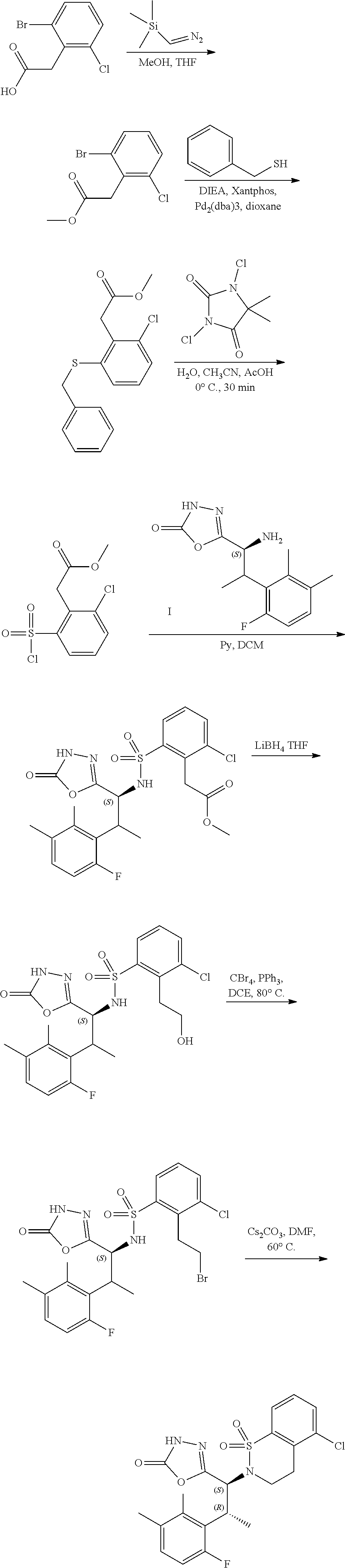

### Step 1: Synthesis of methyl 2-(2-bromo-6-chlorophenyl) acetate

In a 40 mL round-bottom flask were added (2-bromo-6-chlorophenyl) acetic acid (3 g, 12. mmol, 1 equiv), trimethylsilyldiazomethane (2.75 g, 24 mmol, 2.0 equiv), THF (30 mL) and MeOH (10 mL) at room temperature. Then trimethylsilyldiazomethane (2.75 g, 24 mmol, 2.0 equiv) was added at 0° C. The resulting mixture was stirred 3 h at room temperature under nitrogen atmosphere. The resulting mixture was concentrated under reduced pressure. The residue was purified by silica gel column chromatography to afford methyl 2-(2-bromo-6-chlorophenyl) acetate (3 g, 94.7%).

### Step 2: Synthesis of methyl 2-[2-(benzylsulfanyl)-6-chlorophenyl]acetate

In a 40 mL round-bottom flask were added methyl 2-(2-bromo-6-chlorophenyl)acetate (3.2 g, 12.1 mmol, 1 equiv), benzyl mercaptan (1809 mg, 14.6 mmol, 1.2 equiv), DIEA (4708 mg, 36 mmol, 3.0 equiv), Xantphos (1405 mg, 2.4 mmol, 0.2 equiv), Pd₂(dba)₃ (1112 mg, 1.2 mmol, 0.1 equiv) and dioxane (20 mL) at room temperature. The resulting mixture was stirred overnight at 100° C. under nitrogen atmosphere. The resulting mixture was extracted with EtOAc (3×20 mL). The combined organic layers were washed with brine (2×10 mL), dried over anhydrous Na₂SO₄. After filtration, the filtrate was concentrated under reduced pressure. The resulting mixture was filtered, the filter cake was washed with DCM (3×10 mL). The filtrate was concentrated under reduced pressure. The residue was purified by silica gel column chromatography to afford methyl 2-[2-(benzylsulfanyl)-6-chlorophenyl]acetate (3 g, 80.5%).

### Step 3: Synthesis of methyl 2-[2-chloro-6-(chlorosulfonyl)phenyl]acetate

In an 8 mL vial were added methyl 2-[2-(benzylsulfanyl)-6-chlorophenyl]acetate (1000 mg, 3.26 mmol, 1 equiv) and CH₃CN (10 mL) at room temperature. To the above mixture was added H₂O (500 μL) and AcOH (700 μL), followed by 1,3-dichloro-5,5-dimethylimidazolidine-2,4-dione (1284 mg, 6.5 mmol, 2 equiv) dropwise at 0° C. The resulting mixture was stirred for additional 30 min at 0° C. The resulting mixture was extracted with EtOAc. The combined organic layers were washed with brine, dried over anhydrous Na₂SO₄. After filtration, the filtrate was concentrated under reduced pressure. This resulted in methyl 2-[2-chloro-6-(chlorosulfonyl) phenyl]acetate (800 mg, 86.7%).

### Step 4: Synthesis of methyl 2-(2-chloro-6-(N-((1S)-2-(6-fluoro-2,3-dimethylphenyl)-1-(5-oxo-4,5-dihydro-1,3,4-oxadiazol-2-yl) propyl) sulfamoyl) phenyl) acetate

In a 40 mL vial were added methyl 2-[2-chloro-6-(chlorosulfonyl) phenyl]acetate (1000 mg, 3.5 mmol, 1 equiv), Pyridine (2 mL) and DCM (10 mL). To the mixture was added 5-[(1S)-1-amino-2-(6-fluoro-2,3-dimethylphenyl)propyl]-3H-1,3,4-oxadiazol-2-one (937 mg, 3.53 mmol, 1 equiv) at 0° C. The resulting mixture was stirred 2 h at room temperature. The resulting mixture was extracted with DCM. The combined organic layers were washed with brine, dried over anhydrous MgSO₄. After filtration, the filtrate was concentrated under reduced pressure. The residue was purified by reverse flash chromatograph. This resulted in methyl 2-(2-chloro-6-(N-((1S)-2-(6-fluoro-2,3-dimethylphenyl)-1-(5-oxo-4,5-dihydro-1,3,4-oxadiazol-2-yl) propyl) sulfamoyl) phenyl) acetate (500 mg, 27.7%).

### Step 5: Synthesis of 3-chloro-N-[(1S)-2-(6-fluoro-2,3-dimethylphenyl)-1-(5-oxo-4H-1,3,4-oxadiazol-2-yl)propyl]-2-(2-hydroxyethyl)benzenesulfonamide

In a 20 mL vial were added 2-(2-chloro-6-(N-((1S)-2-(6-fluoro-2,3-dimethylphenyl)-1-(5-oxo-4,5-dihydro-1,3,4-oxadiazol-2-yl) propyl) sulfamoyl) phenyl) acetate (600 mg, 1.17 mmol, 1 equiv), THF (3 mL) followed by LiBH₄ 1M (879 μL, 1.76 mmol, 1.5 equiv) dropwise. The resulting mixture was stirred 2 h at room temperature. The resulting mixture was concentrated under vacuum. The residue was purified by reverse flash chromatography. This resulted in 3-chloro-N-[(1S)-2-(6-fluoro-2,3-dimethylphenyl)-1-(5-oxo-4H-1,3,4-oxadiazol-2-yl) propyl]-2-(2-hydroxyethyl) benzenesulfonamide (200 mg, 35.3%).

### Step 6: Synthesis of 2-(2-bromoethyl)-3-chloro-N-[(1S)-2-(6-fluoro-2,3-dimethylphenyl)-1-(5-oxo-4H-1,3,4-oxadiazol-2-yl) propyl]benzenesulfonamide

In a 40 mL vial were added 3-chloro-N-[(1S)-2-(6-fluoro-2,3-dimethylphenyl)-1-(5-oxo-4H-1,3,4-oxadiazol-2-yl) propyl]-2-(2-hydroxyethyl) benzenesulfonamide (400 mg, 0.83 mmol, 1 equiv), DCE (10 mL), PPh₃ (433 mg, 1.65 mmol, 2.0 equiv) and CBr₄ (411 mg, 1.24 mmol, 1.5 equiv). The resulting mixture was stirred overnight at 80° C. The residue was purified by silica gel column chromatography to afford 2-(2-bromoethyl)-3-chloro-N-[(1S)-2-(6-fluoro-2,3-dimethylphenyl)-1-(5-oxo-4H-1,3,4-oxadiazol-2-yl) propyl]benzenesulfonamide (90 mg, 19.9%)

### Step 7: Synthesis of 5-((1S,2R)-1-(5-chloro-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

In a 8 mL vial were added 2-(2-bromoethyl)-3-chloro-N-[(1S)-2-(6-fluoro-2,3-dimethylphenyl)-1-(5-oxo-4H-1,3,4-oxadiazol-2-yl) propyl]benzenesulfonamide (80 mg, 0.15 mmol, 1 equiv), DMF (2 mL) and Cs₂CO₃ (95.3 mg, 0.29 mmol, 2 equiv) at room temperature. The resulting mixture was stirred 30 min at 60° C. The residue was purified by reverse flash chromatography. The crude product was purified by Chiral-Prep-HPLC. This resulted in 5-chloro-2-[(1S,2R)-2-(6-fluoro-2,3-dimethylphenyl)-1-(5-oxo-4H-1,3,4-oxadiazol-2-yl) propyl]-3,4-dihydro-llambda6,2-benzothiazine-1,1-dione (18.1 mg, 26.5%). LCMS (ES, m/z): M−H=464.10. 1H NMR (300 MHz, Methanol-d₄) δ 7.77 (dd, J=7.9, 1.2 Hz, 1H), 7.62 (dd, J=8.1, 1.3 Hz, 1H), 7.48-7.37 (m, 1H), 7.00 (dd, J=8.5, 5.8 Hz, 1H), 6.74 (dd, J=12.1, 8.4 Hz, 1H), 5.52 (dd, J=11.8, 1.7 Hz, 1H), 4.19 (dt, J=14.3, 7.0 Hz, 1H), 4.04 (dt, J=14.8, 6.5 Hz, 1H), 3.94-3.84 (m, 1H), 3.03 (td, J=6.8, 2.4 Hz, 2H), 2.33 (s, 3H), 2.21 (s, 3H), 1.43 (dd, J=6.9, 1.2 Hz, 3H).

### Example 24: 5-((1S,2R)-1-(6-chloro-1,1-dioxido-3,4-dihydro-2H-pyrido[2,3-e][1,2]thiazin-2-yl)-2-(6-fluoro-2,3-dimethylphenyl)propyl)-1,3,4-oxadiazol-2(3H)-one

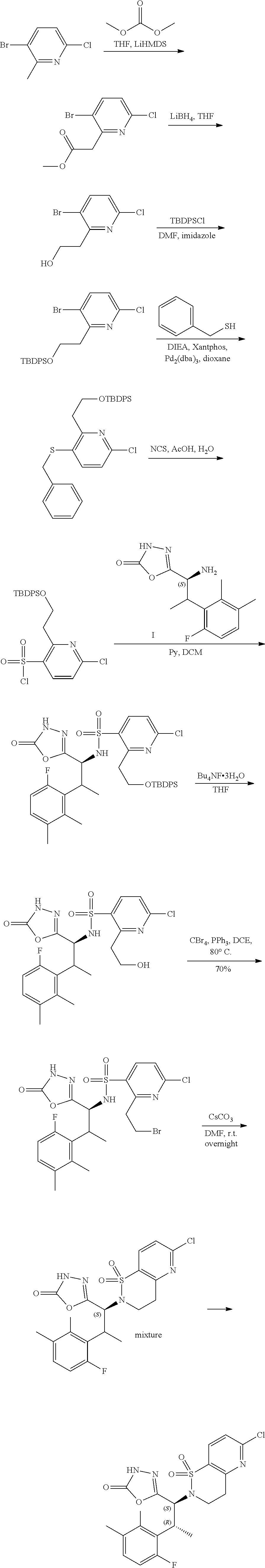

### Step 1: Synthesis of methyl 2-(3-bromo-6-chloropyridin-2-yl) acetate

A solution of 3-bromo-6-chloro-2-methylpyridine (5 g, 24.2 mmol, 1 equiv) and LiHMDS (36.3 mL, 36.3 mmol, 1.5 equiv) in THF was stirred for 1 h at 0° C. under nitrogen atmosphere. To the above mixture was added dimethyl carbonate (3.06 mL, 36.3 mmol, 1.5 equiv) in portions at 0° C. The resulting mixture was stirred 1 h at 0° C. The reaction was quenched by the addition of sat. NH₄Cl (aq.) (200 mL) at room temperature. The resulting mixture was extracted with EtOAc (2×200 mL). The combined organic layers were washed with brine (1×200 mL), dried over anhydrous Na₂SO₄. After filtration, the filtrate was concentrated under reduced pressure. The residue was purified by silica gel column chromatography to afford methyl 2-(3-bromo-6-chloropyridin-2-yl) acetate (5.54 g, 86.5%).

### Step 2: Synthesis of 2-(3-bromo-6-chloropyridin-2-yl) ethanol

In a 50 mL round-bottom flask were added methyl 2-(3-bromo-6-chloropyridin-2-yl) acetate (5.54 g, 20.9 mmol, 1 equiv) and THF (55 mL) at room temperature. To the above mixture was added LiBH₄ (15.7 mL, 31.4 mmol, 1.5 equiv) dropwise at 0° C. The reaction mixture was stirred overnight at 35° C. The resulting mixture was diluted with EtOAc (30 mL). The solution was washed with 1×90 mL of HCl (0.5M). The aqueous phase was reextracted with EtOAc (2×30 mL). The combined organic layers were washed with brine (1×30 mL), dried over anhydrous Na₂SO₄. After filtration, the filtrate was concentrated under reduced pressure. The residue was purified by silica gel column chromatography to afford 2-(3-bromo-6-chloropyridin-2-yl) ethanol (3.36 g, 67.8%).

### Step 3: Synthesis of 3-bromo-2-{2-[(tert-butyldiphenylsilyl) oxy]ethyl}-6-chloropyridine

Into a 20 mL vial were added 2-(3-bromo-6-chloropyridin-2-yl) ethanol (3.36 g, 14.2 mmol, 1 equiv), DMF (33 mL), imidazole (3.87 g, 56.8 mmol, 4 equiv) and TBDPSCl (5.54 mL, 21.3 mmol, 1.5 equiv) at room temperature. The resulting mixture was stirred 1 h at room temperature under air atmosphere. The resulting mixture was quenched with water and extracted with EtOAc (2×50 mL). The combined organic layers were washed with brine (1×50 mL), dried over anhydrous Na₂SO₄. After filtration, the filtrate was concentrated under reduced pressure. The residue was purified by Prep-TLC to afford 3-bromo-2-{2-[(tert-butyldiphenylsilyl) oxy]ethyl}-6-chloropyridine (6.69 g, 99.2%).

### Step 4: Synthesis of 3-(benzylsulfanyl)-2-{2-[(tert-butyldiphenylsilyl) oxy]ethyl}-6-chloropyridine

In a 10 mL vial were added 3-bromo-2-{2-[(tert-butyldiphenylsilyl) oxy]ethyl}-6-chloropyridine (520 mg, 1.1 mmol, 1 equiv), dioxane (3 mL), DIEA (381 μL, 2.19 mmol, 2 equiv), xantphos (63 mg, 0.11 mmol, 0.1 equiv), Pd₂(dba)₃ (50 mg, 0.06 mmol, 0.05 equiv) and benzyl mercaptan (166 μL, 1.42 mmol, 1.3 equiv) at room temperature. The resulting mixture was stirred 3 h at 85° C. under nitrogen atmosphere. The resulting mixture was filtered, the filter cake was washed with EtOAc (3×10 mL). The filtrate was concentrated under reduced pressure. The residue was purified by Prep-TLC to afford 3-(benzylsulfanyl)-2-{2-[(tert-butyldiphenylsilyl) oxy]ethyl}-6-chloropyridine (210 mg, 37%).

### Step 5: Synthesis of 2-(2-((tert-butyldiphenylsilyl) oxy) ethyl)-6-chloropyridine-3-sulfonyl chloride

Into a 25 mL round-bottom flask were added 3-(benzylsulfanyl)-2-{2-[(tert-butyldiphenylsilyl) oxy]ethyl}-6-chloropyridine (1.5 g, 2.90 mmol, 1 equiv), AcOH (13.5 mL) and H₂O (4.50 mL) at room temperature. To the above mixture was added NCS (1.16 g, 8.69 mmol, 3 equiv) in portions at 0° C. The reaction mixture was stirred for additional 2 h at room temperature. The resulting mixture was extracted with EtOAc (2×20 mL). The combined organic layers were washed with brine (1×10 mL), dried over anhydrous Na₂SO₄. After filtration, the filtrate was concentrated under reduced pressure. The residue was purified by silica gel column chromatography to afford 2-[2-[(tert-butyldiphenylsilyl) oxy]ethyl]-6-chloropyridine-3-sulfonyl chloride (950 mg, 66.4%).

### Step 6: Synthesis of 2-(2-((tert-butyldiphenylsilyl) oxy) ethyl)-6-chloro-N-((1S)-2-(6-fluoro-2,3-dimethylphenyl)-1-(5-oxo-4,5-dihydro-1,3,4-oxadiazol-2-yl) propyl)pyridine-3-sulfonamide

In a 20 mL vial were added 2-[2-[(tert-butyldiphenylsilyl) oxy]ethyl]-6-chloropyridine-3-sulfonyl chloride (0.93 g, 1.89 mmol, 1 equiv) and pyridine (3 mL) at room temperature. To the above mixture was added 5-[(1S)-1-amino-2-(6-fluoro-2,3-dimethylphenyl) propyl]-3H-1,3,4-oxadiazol-2-one (500 mg, 1.89 mmol, 1 equiv) in DCM (3 mL) dropwise at 0° C. The resulting mixture was stirred for additional 1 h at room temperature. The residue was purified by reverse flash chromatography. This resulted in 2-(2-((tert-butyldiphenylsilyl) oxy) ethyl)-6-chloro-N-((1S)-2-(6-fluoro-2,3-dimethylphenyl)-1-(5-oxo-4,5-dihydro-1,3,4-oxadiazol-2-yl) propyl)pyridine-3-sulfonamide (840 mg, 61.6%).

### Step 7: Synthesis of 6-chloro-N-[(1S)-2-(6-fluoro-2,3-dimethylphenyl)-1-(5-oxo-4H-1,3,4-oxadiazol-2-yl) propyl]-2-(2-hydroxyethyl) pyridine-3-sulfonamide

In a 50 mL round-bottom flask were added 2-(2-((tert-butyldiphenylsilyl) oxy) ethyl)-6-chloro-N-((1S)-2-(6-fluoro-2,3-dimethylphenyl)-1-(5-oxo-4,5-dihydro-1,3,4-oxadiazol-2-yl) propyl)pyridine-3-sulfonamide (848 mg, 1.17 mmol, 1 equiv) in THF (17 mL) and TBAF (367 mg, 1.41 mmol, 1.2 equiv) at room temperature. The resulting mixture was stirred overnight at room temperature under air atmosphere. The residue was purified by reverse flash chromatography. This resulted in 6-chloro-N-[(1S)-2-(6-fluoro-2,3-dimethylphenyl)-1-(5-oxo-4H-1,3,4-oxadiazol-2-yl) propyl]-2-(2-hydroxyethyl) pyridine-3-sulfonamide (464 mg, 81.6%).

### Step 8: Synthesis of 6-chloro-2-(2-chloroethyl)-N-[(1S)-2-(6-fluoro-2,3-dimethylphenyl)-1-(5-oxo-4H-1,3,4-oxadiazol-2-yl) propyl]pyridine-3-sulfonamide

In a 10 mL round-bottom flask were added 6-chloro-N-[(1S)-2-(6-fluoro-2,3-dimethylphenyl)-1-(5-oxo-4H-1,3,4-oxadiazol-2-yl)propyl]-2-(2-hydroxyethyl)pyridine-3-sulfonamide (464 mg, 0.96 mmol, 1 equiv), DCE (2 mL), CBr₄ (470 mg, 1.44 mmol, 1.5 equiv) and PPh₃ (501 mg, 1.91 mmol, 2 equiv) at room temperature. The reaction mixture was stirred for 1 h at 70° C. under air atmosphere. The resulting mixture was concentrated under reduced pressure. The residue was purified by reverse flash chromatography. This resulted in 6-chloro-2-(2-chloroethyl)-N-[(1S)-2-(6-fluoro-2,3-dimethylphenyl)-1-(5-oxo-4H-1,3,4-oxadiazol-2-yl) propyl]pyridine-3-sulfonamide (278 mg, 57.7%).

### Step 9: Synthesis of 5-((1S,2R)-1-(6-chloro-1,1-dioxido-3,4-dihydro-2H-pyrido[2,3-e][1,2]thiazin-2-yl)-2-(6-fluoro-2,3-dimethylphenyl) propyl)-1,3,4-oxadiazol-2(3H)-one

In a 10 mL vial were added 2-(2-bromoethyl)-6-chloro-N-[(1S)-2-(6-fluoro-2,3-dimethylphenyl)-1-(5-oxo-4H-1,3,4-oxadiazol-2-yl) propyl]pyridine-3-sulfonamide (136 mg, 0.25 mmol, 1 equiv), Cs₂CO₃ (360 mg, 1.10 mmol, 2 equiv), DMF (4.1 mL) at room temperature. The resulting mixture was stirred overnight at room temperature under air atmosphere. The residue was purified by reverse flash chromatography. This resulted in 5-((1S)-1-(6-chloro-1,1-dioxido-3,4-dihydro-2H-pyrido[2,3-e][1,2]thiazin-2-yl)-2-(6-fluoro-2,3-dimethylphenyl) propyl)-1,3,4-oxadiazol-2(3H)-one (150 mg, 58.2%). The product was further purified by reverse flash chromatography. This resulted in 5-((1S,2R)-1-(6-chloro-1,1-dioxido-3,4-dihydro-2H-pyrido[2,3-e][1,2]thiazin-2-yl)-2-(6-fluoro-2,3-dimethylphenyl) propyl)-1,3,4-oxadiazol-2(3H)-one (33.4 mg, 60.42%). LCMS (ES, m/z) [M+H]+=467.05. ¹H NMR (300 MHz, Methanol-d₄) δ 8.17 (d, J=8.3 Hz, 1H), 7.50 (d, J=8.4 Hz, 1H), 7.01 (dd, J=8.5, 5.7 Hz, 1H), 6.75 (dd, J=12.1, 8.4 Hz, 1H), 5.55 (dd, J=11.7, 1.9 Hz, 1H), 4.19 (dt, J=14.7, 7.2 Hz, 1H), 4.05 (ddd, J=15.1, 6.9, 5.6 Hz, 1H), 3.90 (dtd, J=12.7, 7.6, 6.1 Hz, 1H), 3.21 (dt, J=18.6, 6.3 Hz, 1H), 2.94 (dt, J=18.6, 7.2 Hz, 1H), 2.34 (s, 3H), 2.22 (s, 3H), 1.43 (dd, J=7.0, 1.2 Hz, 3H).

### Example 25: 5-((1S,2R)-1-(6-chloro-4-hydroxy-4-methyl-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one

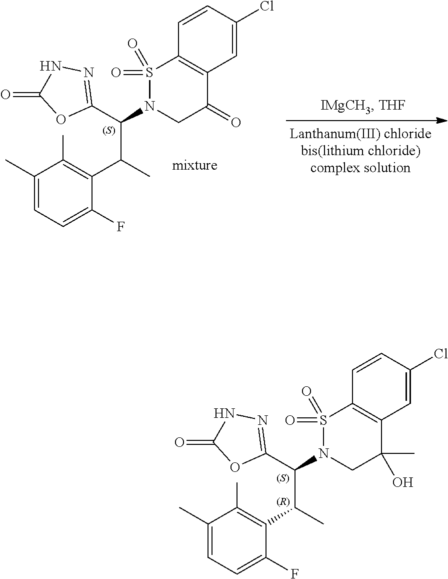

To a stirred mixture of Example 16 (20 mg, 0.042 mmol, 1 equiv) and Lanthanum (III) chloride bis (lithium chloride) complex solution (81 uL, 0.042 mmol, 1 equiv) in THF (0.80 mL) were added iodo(methyl)magnesium (417 μL, 0.42 mmol, 10 equiv) dropwise at 0° C. under nitrogen atmosphere. The resulting mixture was stirred for 1 h at room temperature under nitrogen atmosphere. The reaction was quenched by the addition of sat. NH₄Cl (aq.) (2 mL) at 0° C. The resulting mixture was extracted with EtOAc (3×3 mL). The combined organic layers were washed with brine (1×10 mL), dried over anhydrous Na₂SO₄. After filtration, the filtrate was concentrated under reduced pressure. This resulted in 5-((1S,2R)-1-(6-chloro-4-hydroxy-4-methyl-1,1-dioxido-3,4-dihydro-2H-benzo[e][1,2]thiazin-2-yl)-2-(6-fluoro-2,3-dimethylphenyl)propyl)-1,3,4-oxadiazol-2(3H)-one (20 mg, 97%). The crude product (20 mg) was purified by Chiral-Prep-HPLC. This resulted in 6-chloro-2-[(1S,2R)-2-(6-fluoro-2,3-dimethylphenyl)-1-(5-oxo-4H-1,3,4-oxadiazol-2-yl)propyl]-4-hydroxy-4-methyl-3H-llambda6,2-benzothiazine-1,1-dione (3.4 mg, 16.5%). LC-MS (ES, m/z):M−H=494.05. 1H NMR (400 MHz, Methanol-d₄) δ 7.75-7.71 (d, J=8.4 Hz, 1H), 7.71-7.70 (d, J=2.2 Hz, 1H), 7.51-7.49 (dd, J=8.5, 2.1 Hz, 1H), 7.00-6.96 (dd, J=8.4, 5.7 Hz, 1H), 6.75-6.70 (dd, J=11.9, 8.4 Hz, 1H), 5.63-5.60 (d, J=11.4 Hz, 1H), 3.95 (d, J=14.6 Hz, 1H), 3.97-3.83 (d, J=14.7 Hz, 1H), 3.78-3.63 (d, J=9.0 Hz, 2H), 2.35 (s, 3H), 2.22 (s, 3H), 1.64 (s, 3H), 1.45-1.44 (d, J=6.8 Hz, 3H).

### Example 26: 5-((1S)-1-((4S,5R)-7-chloro-4,5-dihydroxy-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one

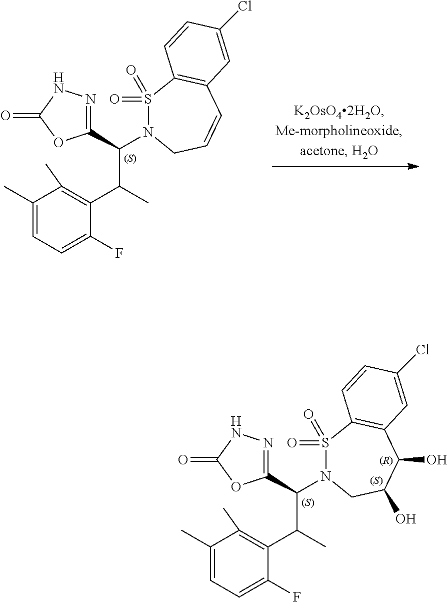

Into a 100 mL round-bottom flask were added 5-((1S)-1-(7-chloro-1,1-dioxidobenzo[f][1,2]thiazepin-2(3H)-yl)-2-(6-fluoro-2,3-dimethylphenyl)propyl)-1,3,4-oxadiazol-2(3H)-one (100 mg, 0.21 mmol, 1 equiv), Acetone (2 mL, NMO (73.5 mg, 0.63 mmol, 3 equiv), K₂OsO₄·2H₂O (38.6 mg, 0.10 mmol, 0.5 equiv) and H₂O (2 mL) at room temperature. The resulting mixture was stirred overnight at room temperature. The resulting mixture was filtered, the filter cake was washed with EtOAc (2×10 mL). The filtrate was concentrated under reduced pressure. The residue was purified by reverse flash chromatography. The residue was purified by reverse flash chromatography. This resulted in (5-((1S)-1-((4S,5R)-7-chloro-4,5-dihydroxy-1,1-dioxido-4,5-dihydrobenzo[f][1,2]thiazepin-2(3H)-yl)-2-(6-fluoro-2,3-dimethylphenyl)propyl)-1,3,4-oxadiazol-2(3H)-one (4.0 mg, 3.7%). LCMS (ES, m/z): [M−H]*=510.10. ¹H NMR (300 MHz, Methanol-d₄) δ 7.96 (s, 1H), 7.82 (dd, J=16.9, 8.3 Hz, 1H), 7.43 (d, J=8.9 Hz, 1H), 7.15-6.86 (m, 1H), 6.70 (dd, J=12.0, 8.4 Hz, 1H), 5.74-5.59 (m, 2H), 4.23-3.96 (m, 2H), 3.89-3.51 (m, 2H), 2.25 (d, J=24.2 Hz, 6H), 1.39-1.28 (m, 1H), 0.90 (s, 2H).

### Example 27: 5-((1S,2R)-1-(6-chloro-1,1-dioxido-3,4-dihydro-2H-pyrido[4,3-e][1,2]thiazin-2-yl)-2-(6-fluoro-2,3-dimethylphenyl) propyl)-1,3,4-oxadiazol-2(3H)-one

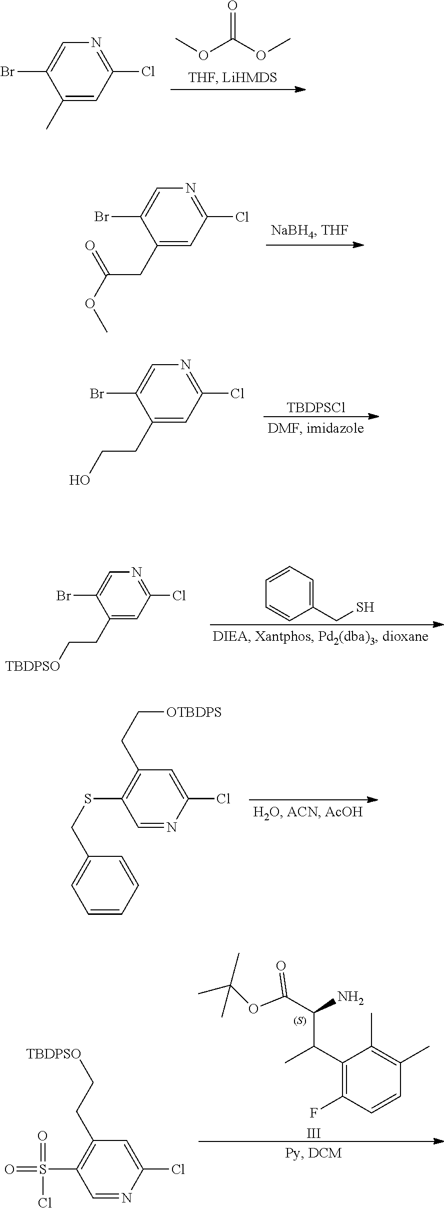

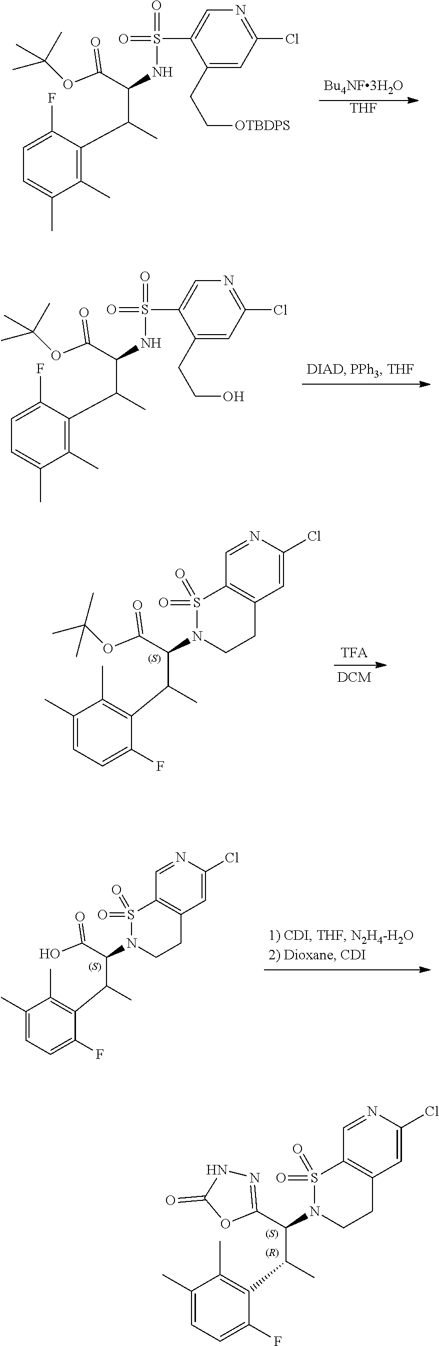

### Step 1: Synthesis of methyl 2-(5-bromo-2-chloropyridin-4-yl) acetate

A solution of 3-bromo-6-chloro-2-methylpyridine (5 g, 24.2 mmol, 1 equiv) and LiHMDS (51 mL, 51 mmol, 1.5 equiv) in THF was stirred for 1 h at 0° C. under nitrogen atmosphere. To the above mixture was added dimethyl carbonate (4.3 mL, 51 mmol, 1.5 equiv) in portions at 0° C. The resulting mixture was stirred 1 h at 0° C. The reaction was quenched by the addition of sat. NH₄Cl (aq.) (200 mL) at room temperature. The resulting mixture was extracted with EtOAc (3×100 mL). The combined organic layers were washed with brine (1×150 mL), dried over anhydrous Na₂SO₄. After filtration, the filtrate was concentrated under reduced pressure. The residue was purified by reverse flash chromatography. This resulted in methyl 2-(5-bromo-2-chloropyridin-4-yl) acetate (4.2 g, 46.3%).

### Step 2: Synthesis of 5-bromo-4-(2-((tert-butyldiphenylsilyl) oxy) ethyl)-2-chloropyridine

Into a 40 mL vial were added 2-(5-bromo-2-chloropyridin-4-yl) ethanol (1.91 g, 8.1 mmol, 1 equiv), DMF (38 mL), imidazole (2.20 g, 32.3 mmol, 4 equiv) and TBDPSCl (4.20 mL, 16. 2 mmol, 2 equiv) at room temperature. The resulting mixture was stirred for 2 h at 35° C. under air atmosphere. The residue was purified by reverse flash chromatography. This resulted in 5-bromo-4-(2-((tert-butyldiphenylsilyl) oxy) ethyl)-2-chloropyridine (3.66 g, 95.4%).

### Step 3: Synthesis of 5-(benzylthio)-4-(2-((tert-butyldiphenylsilyl) oxy) ethyl)-2-chloropyridine

Into a 20 mL vial were added 5-bromo-4-{2-[(tert-butyldiphenylsilyl) oxy]ethyl}-2-chloropyridine (3.7 g, 7.8 mmol, 1 equiv), dioxane (37 mL), Xantphos (450 mg, 0.8 mmol, 0.1 equiv), Pd₂(dba)₃ (356 mg, 0.39 mmol, 0.05 equiv) and benzyl mercaptan (1.1 mL, 9.3 mmol, 1.2 equiv) at room temperature. The resulting mixture was stirred overnight at 80° C. under nitrogen atmosphere. The resulting mixture was filtered, the filter cake was washed with EtOAc (2×30 mL). The filtrate was concentrated under reduced pressure. The residue was purified by silica gel column chromatography to afford 5-(benzylthio)-4-(2-((tert-butyldiphenylsilyl) oxy) ethyl)-2-chloropyridine (1.6 g, 38.5%).

### Step 4: Synthesis of 4-(2-((tert-butyldiphenylsilyl) oxy) ethyl)-6-chloropyridine-3-sulfonyl chloride

Into a 100 mL round-bottom flask were added 5-(benzylthio)-4-(2-((tert-butyldiphenylsilyl) oxy) ethyl)-2-chloropyridine (1.55 g, 3 mmol, 1 equiv), AcOH (15 mL) and H₂O (5 mL) at room temperature. To the above mixture was added N-chlorosuccinimide (1.2 g, 9 mmol, 3 equiv) in portions at 0° C. The resulting mixture was stirred for additional 2 h at room temperature. The resulting mixture was extracted with EtOAc (3×50 mL). The combined organic layers were washed with brine (1×100 mL), dried over anhydrous Na₂SO₄. After filtration, the filtrate was concentrated under reduced pressure. This resulted in 4-{2-[(tert-butyldiphenylsilyl) oxy]ethyl}-6-chloropyridine-3-sulfonyl chloride (1.5 g, 100%).

### Step 5: Synthesis of tert-butyl (2S)-2-((4-(2-((tert-butyldiphenylsilyl) oxy) ethyl)-6-chloropyridine)-3-sulfonamido)-3-(6-fluoro-2,3-dimethylphenyl) butanoate

Into a 8 mL vial were added intermediate III 5-[(1S)-1-amino-2-(6-fluoro-2,3-dimethylphenyl) propyl]-3H-1,3,4-oxadiazol-2-one (500 mg, 1.8 mmol, 1.00 equiv) and Pyridine (719 μL, 8.9 mmol, 5 equiv) at room temperature. To the above mixture was added 4-{2-[(tert-butyldiphenylsilyl) oxy]ethyl}-6-chloropyridine-3-sulfonyl chloride (1255 mg, 1.8 mmol, 1 equiv, 70%) in DCM (2 mL) dropwise at 0° C. The resulting mixture was stirred overnight at room temperature. The residue was purified by silica gel column chromatography to afford tert-butyl (2S)-2-((4-(2-((tert-butyldiphenylsilyl) oxy) ethyl)-6-chloropyridine)-3-sulfonamido)-3-(6-fluoro-2,3-dimethylphenyl) butanoate (540 mg, 41%).

### Step 6: Synthesis of tert-butyl (2S)-2-[6-chloro-4-(2-hydroxyethyl) pyridine-3-sulfonamido]-3-(6-fluoro-2,3-dimethylphenyl) butanoate

Into a 100 mL round-bottom flask were added tert-butyl (2S)-2-(4-{2-[(tert-butyldiphenylsilyl) oxy]ethyl}-6-chloropyridine-3-sulfonamido)-3-(6-fluoro-2,3-dimethylphenyl) butanoate (530 mg, 0.72 mmol, 1 equiv), THF (10. mL) and TBAF (281 mg, 1.08 mmol, 1.5 equiv) at room temperature. The resulting mixture was stirred 1 h at room temperature under air atmosphere. The residue was purified by reverse flash chromatography. This resulted in tert-butyl (2S)-2-[6-chloro-4-(2-hydroxyethyl) pyridine-3-sulfonamido]-3-(6-fluoro-2,3-dimethylphenyl) butanoate (200 mg, 55.7%).

### Step 7: Synthesis of tert-butyl (2S)-2-(6-chloro-1,1-dioxido-3,4-dihydro-2H-pyrido[4,3-e][1,2]thiazin-2-yl)-3-(6-fluoro-2,3-dimethylphenyl) butanoate

Into a 10 mL vial were added in tert-butyl (2S)-2-[6-chloro-4-(2-hydroxyethyl) pyridine-3-sulfonamido]-3-(6-fluoro-2,3-dimethylphenyl) butanoate (20 mg, 0.04 mmol, 1 equiv), THF (1.6 mL) and PPh₃ (83.8 mg, 0.32 mmol, 2 equiv) at room temperature. The resulting mixture was stirred for 20 min at room temperature under nitrogen atmosphere. To the above mixture was added DIAD (63.3 μL, 0.32 mmol, 2.00 equiv) dropwise at room temperature. The resulting mixture was stirred for additional 1 h at room temperature. The residue was purified by reverse flash chromatography. This resulted in tert-butyl (2S)-2-(6-chloro-1,1-dioxido-3,4-dihydro-2H-pyrido[4,3-e][1,2]thiazin-2-yl)-3-(6-fluoro-2,3-dimethylphenyl) butanoate (40 mg, 51.9%).

### Step 8: Synthesis of (2S)-2-(6-chloro-1,1-dioxido-3,4-dihydro-2H-pyrido[4,3-e][1,2]thiazin-2-yl)-3-(6-fluoro-2,3-dimethylphenyl)butanoic acid

Into a 25 mL round-bottom flask were tert-butyl (2S)-2-(6-chloro-1,1-dioxido-3,4-dihydro-2H-pyrido[4,3-e][1,2]thiazin-2-yl)-3-(6-fluoro-2,3-dimethylphenyl) butanoate (100 mg, 0.02 mmol, 1 equiv), DCM (0.9 mL) and TFA (0.3 mL) at room temperature. The resulting mixture was stirred for 2 h at room temperature under air atmosphere. The resulting mixture was concentrated under reduced pressure. The residue was purified by reverse flash chromatography. This resulted in (2S)-2-(6-chloro-1,1-dioxido-3,4-dihydro-2H-pyrido[4,3-e][1,2]thiazin-2-yl)-3-(6-fluoro-2,3-dimethylphenyl) butanoic acid (80 mg, 90.5%).

### Step 9: Synthesis of 5-((1S,2R)-1-(6-chloro-1,1-dioxido-3,4-dihydro-2H-pyrido[4,3-e][1,2]thiazin-2-yl)-2-(6-fluoro-2,3-dimethylphenyl)propyl)-1,3,4-oxadiazol-2(3H)-one

Into a 10 mL vial were added 2S)-2-(6-chloro-1,1-dioxido-3,4-dihydro-2H-pyrido[4,3-e][1,2]thiazin-2-yl)-3-(6-fluoro-2,3-dimethylphenyl) butanoic acid (70 mg, 0.16 mmol, 1 equiv), CDI (79.8 mg, 0.49 mmol, 3 equiv) and THF (1.4 mL) at room temperature. The mixture was stirred for 30 min at room temperature under air atmosphere. To the above mixture was added NH₂NH₂·H₂O (23.9 μL, 0.49 mmol, 3 equiv) dropwise at 0° C. The mixture was stirred for additional 30 min at 0° C. The mixture was extracted with EtOAc (2×10 mL). The combined organic layers were washed with brine (1×10 mL), dried over anhydrous Na₂SO₄. After filtration, the filtrate was concentrated under reduced pressure. Into a 10 mL vial were added the above crude product, CDI (79.8 mg, 0.49 mmol, 3 equiv) and dioxane (0.2 mL) at room temperature. The mixture was stirred for 1 h at room temperature under air atmosphere. The resulting mixture was poured into water and extracted with EtOAc (3×5 mL). The combined organic layers were washed with brine, dried over anhydrous Na₂SO₄ and evaporated. The residue was purified by reverse flash chromatography. This resulted in 5-((1S,2R)-1-(6-chloro-1,1-dioxido-3,4-dihydro-2H-pyrido[4,3-e][1,2]thiazin-2-yl)-2-(6-fluoro-2,3-dimethylphenyl)propyl)-1,3,4-oxadiazol-2(3H)-one (21.8 mg, 25.9%). LCMS (ES, m/z): [M−H]+=465.05. ¹H NMR (300 MHz, Methanol-d₄) δ 8.72 (s, 1H), 7.72 (s, 1H), 7.47 (s, 1H), 7.08 (s, 1H), 7.02 (dd, J=8.4, 5.7 Hz, 1H), 6.76 (dd, J=12.1, 8.4 Hz, 1H), 5.57 (dd, J=11.8, 1.8 Hz, 1H), 4.19 (dt, J=14.2, 7.1 Hz, 1H), 4.03 (dt, J=14.2, 6.4 Hz, 1H), 3.88 (dq, J=13.3, 7.4, 6.8 Hz, 1H), 3.18 (dt, J=18.2, 6.6 Hz, 1H), 2.95 (dt, J=18.1, 6.9 Hz, 1H), 2.35 (s, 3H), 2.23 (s, 3H), 1.45 (d, J=6.8 Hz, 3H).

### Example A: RNR Enzyme Activity

A rapid-fire mass spectrometry (RF/MS) assay was used to assess RNR enzyme activity using a 384 well plate and a robotic platform.

The plate layout included two validated reference compounds (Triapine (3-AP) and Hydroxyurea (HU)):
A dose response in duplicate; top concentration: 5 μM (3-AP) and 250 μM (HU), semi-log
dilutions.
Spike wells in triplicate randomly spotted at four concentrations:
   250 μM, 100 μM, 30 μM and 2 μM for HU
   5 μM, 2 μM, 0.6 μM and 0.04 μM for 3-AP

First, the multidrop pipes were saturated for 30 minutes with enzymatic solution. Then 30 μL of Stop solution was distributed in column 24. Next, 15 μL of enzyme was distributed in column 1 to 24. Next, a pre-incubation step of 15 minutes at room temperature occurred, followed by distribution of 15 μL of substrate solution (column 1 to 24). Next, the plate was incubated for 45 minutes at 37° C. 30 μL of Stop solution was distributed to columns 1 to 23.

The final parameters for the enzyme reactions were:
Incubation: 37° C., 45 min
[CDP]: 5 μM; [ATP]: 1 mM; [NADPH]: No
[RNR]final: 50 nM with 1:1 (RNR1:RNR2) ratio
Final volume: 30 μL
Stop solution: 6% HCOOH containing 2 μM of 15

The compounds were screened at concentrations up to 50 μM concentrations and the results are shown in table 2.

TABLE 2

| Ex. | RF/MS - IC₅₀ |
|---|---|
| 1 | D |
| 2 | C |
| 3 | D |
| 4 | D |
| 5 | D |
| 6 | C |
| 7 | A |
| 8 | D |
| 9 | A |
| 10 | D |
| 11 | B |
| 12 | D |
| 13 | A |
| 14 | C |
| 15 | D |
| 16 | B |
| 17 | D |
| 18 | B |
| 19 | B |
| 20 | A |
| 21 | C |
| 22 | B |
| 23 | B |
| 24 | A |
| 25 | A |
| 26 | A |
| 27 | B |

A: IC₅₀ ≤ 100 nM;
B: 100 nM < IC₅₀ ≤ 1 μM
C: 1 μM < IC₅₀ ≤ 10 μM
D: 10 μM < IC₅₀ ≤ 100 μM
NT: not tested

### Example B: Alphalisa Assay

Colo320 DM cells (ATCC #CCL-220, derived from human colorectal adenocarcinoma, Dukes' type C) were seeded on a 96-well, cell culture treated assay plate at a density of 50,000 cells/well in 200 μL of RPMI-1640 media supplemented with 10% Fetal Bovine Serum and incubated at 37 degrees Celsius overnight. The following day, test compound dilutions were added directly to the plated cells by a Tecan digital dispenser to a final DMSO concentration of <0.5%. and incubated at 37 degrees Celsius overnight (approximately 16 hours). The following day all cull culture media was removed from the cells. 75 μL of 1× AlphaLisa lysis buffer was added to each well and plates were agitated on a shaker for 30 minutes at room temperature. The lysis of cells and detection of pCHK1 (S345) were performed with regents contained within the AlphaLisa Sure Fire assay kit (Perkin Elmer #ALSU-PCHK1-A) according to the manufacturer's instructions. 10 μL of each lysate was then transferred to a white, 384-well assay plate (Perkin Elmer #6008280). 5 μL of Acceptor mix was then added to each well of lysate in the white, 384-well assay plate and incubated in the dark at room temperature for 60 minutes. 5 μL of Donor mix was then added to each well of the white, 384-well assay plate in subdued light and incubated at room temperature for 60 minutes. Plates were read on an Alpha Technology-compatible plate reader using standard AlphaLisa settings.

The results are shown in table 3.

TABLE 3

| Example | Alphalisa IC₅₀ |
|---|---|
| 1 | D |
| 2 | D |
| 3 | D |
| 4 | D |
| 5 | D |
| 6 | D |
| 7 | A |
| 8 | D |
| 9 | A |
| 10 | D |
| 11 | D |
| 12 | D |
| 13 | B |
| 14 | D |
| 15 | D |
| 16 | B |
| 17 | D |
| 18 | C |
| 19 | C |
| 20 | A |
| 21 | D |
| 22 | B |
| 23 | C |
| 24 | A |
| 25 | C |
| 26 | C |
| 27 | D |

A: IC₅₀ ≤ 5 μM;
B: 5 μM < IC₅₀ ≤ 10 μM
C: 10 μM < IC₅₀ ≤ 50 μM
D: 50 μM < IC₅₀
NT: not tested

### Example C: Pharmaceutical Compositions

### Example C1: Parenteral Composition

To prepare a parenteral pharmaceutical composition suitable for administration by injection, 100 mg of a water-soluble salt of a compound described herein is dissolved in DMSO and then mixed with 10 mL of 0.9% sterile saline. The mixture is incorporated into a dosage unit form suitable for administration by injection.

### Example C2: Oral Composition

To prepare a pharmaceutical composition for oral delivery, 100 mg of a compound described herein is mixed with 750 mg of starch. The mixture is incorporated into an oral dosage unit for, such as a hard gelatin capsule, which is suitable for oral administration.

### Example C3: Sublingual (Hard Lozenge) Composition

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

The examples and embodiments described herein are for illustrative purposes only and in some embodiments, various modifications or changes are to be included within the purview of disclosure and scope of the appended claims.

What is claimed is:

1. A compound of Formula (I), or a pharmaceutically acceptable salt, solvate, tautomer, or stereoisomer thereof: 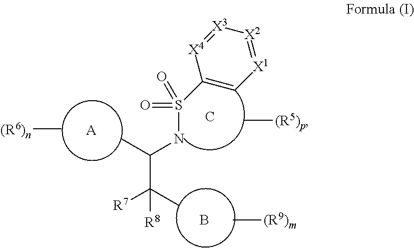
wherein:
X¹ is N or CR¹;
X² is N or CR²;
X³ is N or CR³;
X⁴ is N or CR⁴;
R¹, R², R³, and R⁴ are independently hydrogen, deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NRC(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
Ring C is a 4- to 8-membered heterocycloalkyl optionally comprising 1 or 2 additional heteroatoms selected from the group consisting of O, S, and N;
each R⁵ is independently deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, or C₁-C₆aminoalkyl;
or 2 R⁵ on the same carbon are taken together to form an oxo;
p is 0-4;
Ring A is a 5-membered ring comprising 1-4 heteroatoms selected from the group consisting of O, S, and N;
each R⁶ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁶ on the same atom are taken together to form an oxo;
n is 0-3;
R⁷ is hydrogen, deuterium, halogen, —CN, —NO₂, —OH, —OR^{a}, C₁-C₆alkyl, C₁-C₆haloalkyl, C₁-C₆deuteroalkyl, C₁-C₆hydroxyalkyl, C₁-C₆aminoalkyl, cycloalkyl, heterocycloalkyl, aryl, or heteroaryl;
R⁸ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
Ring B is cycloalkyl, heterocycloalkyl, aryl, or heteroaryl;
each R⁹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NRC(═O)NR^{c}R^{d}, —NR^{b}C(═O)R^{a}, —NRC(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more R^{9a};
or two R⁹ on the same atom are taken together to form an oxo;
each R^{9a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally and independently substituted with one or more of deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{9a} on the same atom are taken together to form an oxo;
m is 0-5;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl); wherein each alkyl, alkenyl, alkynyl, cycloalkyl, heterocycloalkyl, aryl, and heteroaryl is independently optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more substituents that is oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, C₁-C₆heteroalkyl.

2. The compound of claim 1, or a pharmaceutically acceptable salt, solvate, tautomer, or stereoisomer thereof, wherein:
Ring A is a 5-membered ring comprising 2-4 heteroatoms selected from the group consisting of 0, S, and N.

3. The compound of claim 1, or a pharmaceutically acceptable salt, solvate, tautomer, or stereoisomer thereof, wherein:
Ring A is a 5-membered ring comprising 3 or 4 heteroatoms selected from the group consisting of O, S, and N.

4. The compound of claim 1, or a pharmaceutically acceptable salt, solvate, tautomer, or stereoisomer thereof, wherein:
Ring A is a 5-membered ring comprising 3 heteroatoms selected from the group consisting of 0, S, and N.

5. The compound of claim 1, or a pharmaceutically acceptable salt, solvate, tautomer, or stereoisomer thereof, wherein:
Ring A is a 5-membered ring comprising 3 heteroatoms selected from the group consisting of 0 and N.

6. The compound of claim 1, or a pharmaceutically acceptable salt, solvate, tautomer, or stereoisomer thereof, wherein:
Ring A is a triazole or tetrazole.

7. The compound of claim 1, or a pharmaceutically acceptable salt, solvate, tautomer, or stereoisomer thereof, wherein:
Ring A is a triazole.

8. The compound of claim 1, or a pharmaceutically acceptable salt, solvate, tautomer, or stereoisomer thereof, wherein:
Ring A is a tetrazole.

9. The compound of claim 1, or a pharmaceutically acceptable salt, solvate, tautomer, or stereoisomer thereof, wherein:
Ring A is a 2,3-dihydro-1,3,4-oxadiazole.

10. The compound of any one of claims 1-9, or a pharmaceutically acceptable salt, solvate, tautomer, or stereoisomer thereof, wherein:
each R⁶ is independently deuterium, halogen, —CN, —OH, —OR^{a}, —NR^{c}R^{d}, C₁-C₆alkyl, C₁-C₆haloalkyl, or C₁-C₆deuteroalkyl; or two R⁶ on the same atom are taken together to form an oxo.

11. The compound of any one of claims 1-10, or a pharmaceutically acceptable salt, solvate, tautomer, or stereoisomer thereof, wherein:
each R⁶ is independently deuterium, halogen, or C₁-C₆alkyl; or two R⁶ on the same atom are taken together to form an oxo.

12. The compound of any one of claims 1-11, or a pharmaceutically acceptable salt, solvate, tautomer, or stereoisomer thereof, wherein:
two R⁶ on the same atom are taken together to form an oxo.

13. The compound of any one of claims 1-12, or a pharmaceutically acceptable salt, solvate, tautomer, or stereoisomer thereof, wherein:
n is 0.

14. The compound of any one of claims 1-12, or a pharmaceutically acceptable salt, solvate, tautomer, or stereoisomer thereof, wherein:
n is 1.

15. The compound of any one of claims 1-12, or a pharmaceutically acceptable salt, solvate, tautomer, or stereoisomer thereof, wherein:
n is 2.

16. The compound of any one of claims 1-5 or 9-15, or a pharmaceutically acceptable salt, solvate, tautomer, or stereoisomer thereof, wherein the compound of Formula (I) is of Formula (Ia): 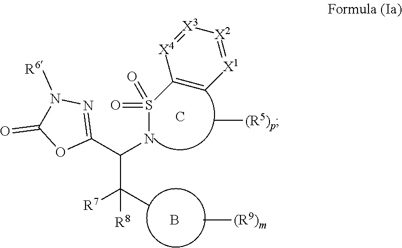
wherein
R^{6′} is hydrogen or C₁-C₆alkyl.

17. The compound of claim 16, or a pharmaceutically acceptable salt, solvate, tautomer, or stereoisomer thereof, wherein:
R^{6′} is hydrogen.

18. The compound of any one of claims 1-17, or a pharmaceutically acceptable salt, solvate, tautomer, or stereoisomer thereof, wherein:
X¹ is N.

19. The compound of any one of claims 1-17, or a pharmaceutically acceptable salt, solvate, tautomer, or stereoisomer thereof, wherein:
X¹ is CR¹.

20. The compound of any one of claims 1-19, or a pharmaceutically acceptable salt, solvate, tautomer, or stereoisomer thereof, wherein:
X² is N.

21. The compound of any one of claims 1-19, or a pharmaceutically acceptable salt, solvate, tautomer, or stereoisomer thereof, wherein:
X² is CR².

22. The compound of any one of claims 1-21, or a pharmaceutically acceptable salt, solvate, tautomer, or stereoisomer thereof, wherein:
X³ is N.

23. The compound of any one of claims 1-21, or a pharmaceutically acceptable salt, solvate, tautomer, or stereoisomer thereof, wherein:
X³ is CR³.

24. The compound of any one of claims 1-23, or a pharmaceutically acceptable salt, solvate, tautomer, or stereoisomer thereof, wherein:
X⁴ is N.

25. The compound of any one of claims 1-23, or a pharmaceutically acceptable salt, solvate, tautomer, or stereoisomer thereof, wherein:
X⁴ is CR⁴.

26. The compound of any one of claims 1-25, or a pharmaceutically acceptable salt, solvate, tautomer, or stereoisomer thereof, wherein:
Ring C is a 5- to 7-membered heterocycloalkyl optionally comprising 1 or 2 additional heteroatoms selected from the group consisting of O, S, and N.

27. The compound of any one of claims 1-25, or a pharmaceutically acceptable salt, solvate, tautomer, or stereoisomer thereof, wherein:
Ring C is a 5- to 6-membered heterocycloalkyl optionally comprising 1 or 2 additional heteroatoms selected from the group consisting of O, S, and N.

28. The compound of any one of claims 1-25, or a pharmaceutically acceptable salt, solvate, tautomer, or stereoisomer thereof, wherein:
Ring C is a 5-membered heterocycloalkyl optionally comprising 1 or 2 additional heteroatoms selected from the group consisting of O, S, and N.

29. The compound of any one of claims 1-25, or a pharmaceutically acceptable salt, solvate, tautomer, or stereoisomer thereof, wherein:
Ring C is a 6-membered heterocycloalkyl optionally comprising 1 or 2 additional heteroatoms selected from the group consisting of O, S, and N.

30. The compound of any preceding claims, or a pharmaceutically acceptable salt, solvate, tautomer, or stereoisomer thereof, wherein the compound of Formula (I) or (Ia) is of Formula (Ib): 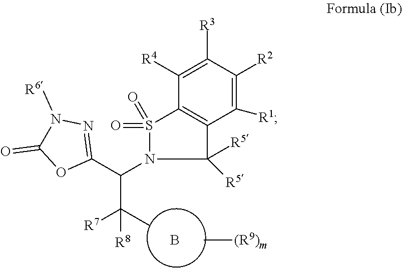
wherein
R^{6′} is hydrogen or C₁-C₆alkyl; and
each R^{5′} is independently hydrogen or R⁵.

31. The compound of any preceding claims, or a pharmaceutically acceptable salt, solvate, tautomer, or stereoisomer thereof, wherein the compound of Formula (I) or (Ia) is of Formula (Ic): 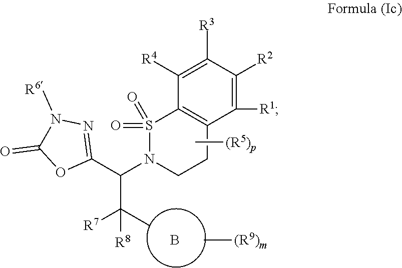
wherein
R^{6′} is hydrogen or C₁-C₆alkyl.

32. The compound of any preceding claims, or a pharmaceutically acceptable salt, solvate, tautomer, or stereoisomer thereof, wherein the compound of Formula (I) or (Ia) is of Formula (Id): 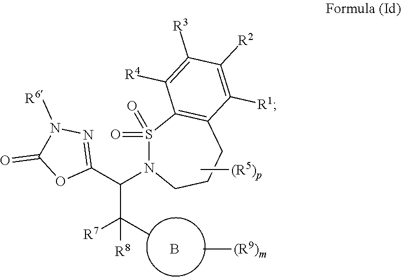
wherein
R^{6′} is hydrogen or C₁-C₆alkyl.

33. The compound of any one of claims 16-32, or a pharmaceutically acceptable salt, solvate, tautomer, or stereoisomer thereof, wherein:
R^{6′} is hydrogen.

34. The compound of any one of claims 1-33, or a pharmaceutically acceptable salt, solvate, tautomer, or stereoisomer thereof, wherein:
each R⁵ is independently deuterium, halogen, —OH, —OR^{a}, —NR^{c}R^{d}, C₁-C₆alkyl, or C₁-C₆haloalkyl; or
R⁵ on the same carbon are taken together to form an oxo.

35. The compound of any one of claims 1-34, or a pharmaceutically acceptable salt, solvate, tautomer, or stereoisomer thereof, wherein:
each R⁵ is independently deuterium, halogen, or C₁-C₆alkyl.

36. The compound of any one of claims 1-35, or a pharmaceutically acceptable salt, solvate, tautomer, or stereoisomer thereof, wherein:
p is 0.

37. The compound of any one of claims 1-36, or a pharmaceutically acceptable salt, solvate, tautomer, or stereoisomer thereof, wherein:
p is 1 or 2.

38. The compound of any one of claims 1-37, or a pharmaceutically acceptable salt, solvate, tautomer, or stereoisomer thereof, wherein:
p is 1.

39. The compound of any one of claims 1-37, or a pharmaceutically acceptable salt, solvate, tautomer, or stereoisomer thereof, wherein:
p is 2.

40. The compound of any one of claims 1-39, or a pharmaceutically acceptable salt, solvate, tautomer, or stereoisomer thereof, wherein:
R¹ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

41. The compound of any one of claims 1-40, or a pharmaceutically acceptable salt, solvate, tautomer, or stereoisomer thereof, wherein:
R¹ is hydrogen, deuterium, halogen, —CN, —OH, —OR^{a}, —NR^{c}R^{d}, C₁-C₆alkyl, or C₁-C₆haloalkyl.

42. The compound of any one of claims 1-41, or a pharmaceutically acceptable salt, solvate, tautomer, or stereoisomer thereof, wherein:
R¹ is hydrogen, halogen, —OH, —OW, C₁-C₆alkyl, or C₁-C₆haloalkyl.

43. The compound of any one of claims 1-42, or a pharmaceutically acceptable salt, solvate, tautomer, or stereoisomer thereof, wherein:
R² is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

44. The compound of any one of claims 1-43, or a pharmaceutically acceptable salt, solvate, tautomer, or stereoisomer thereof, wherein:
R² is hydrogen, deuterium, halogen, —CN, —OH, —OR^{a}, —NR^{c}R^{d}, C₁-C₆alkyl, or C₁-C₆haloalkyl.

45. The compound of any one of claims 1-44, or a pharmaceutically acceptable salt, solvate, tautomer, or stereoisomer thereof, wherein:
R² is hydrogen, halogen, —OH, —OR^{a}, C₁-C₆alkyl, or C₁-C₆haloalkyl.

46. The compound of any one of claims 1-45, or a pharmaceutically acceptable salt, solvate, tautomer, or stereoisomer thereof, wherein:
R³ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

47. The compound of any one of claims 1-46, or a pharmaceutically acceptable salt, solvate, tautomer, or stereoisomer thereof, wherein:
R³ is hydrogen, deuterium, halogen, —CN, —OH, —OW, —NR^{c}R^{d}, C₁-C₆alkyl, or C₁-C₆haloalkyl.

48. The compound of any one of claims 1-47, or a pharmaceutically acceptable salt, solvate, tautomer, or stereoisomer thereof, wherein:
R³ is hydrogen, halogen, —OH, —OW, C₁-C₆alkyl, or C₁-C₆haloalkyl.

49. The compound of any one of claims 1-48, or a pharmaceutically acceptable salt, solvate, tautomer, or stereoisomer thereof, wherein:
R⁴ is hydrogen, deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

50. The compound of any one of claims 1-49, or a pharmaceutically acceptable salt, solvate, tautomer, or stereoisomer thereof, wherein:
R⁴ is hydrogen, deuterium, halogen, —CN, —OH, —OR^{a}, —NR^{c}R^{d}, C₁-C₆alkyl, or C₁-C₆haloalkyl.

51. The compound of any one of claims 1-50, or a pharmaceutically acceptable salt, solvate, tautomer, or stereoisomer thereof, wherein:
R⁴ is hydrogen, halogen, —OH, —OW, C₁-C₆alkyl, or C₁-C₆haloalkyl.

52. The compound of any one of claims 1-51, or a pharmaceutically acceptable salt, solvate, tautomer, or stereoisomer thereof, wherein:
R⁷ is hydrogen, deuterium, halogen, C₁-C₆alkyl, C₁-C₆haloalkyl, C₁-C₆deuteroalkyl, C₁-C₆hydroxyalkyl, C₁-C₆aminoalkyl, cycloalkyl, or heterocycloalkyl.

53. The compound of any one of claims 1-52, or a pharmaceutically acceptable salt, solvate, tautomer, or stereoisomer thereof, wherein:
R⁷ is hydrogen, deuterium, halogen, C₁-C₆alkyl, C₁-C₆haloalkyl, or C₁-C₆deuteroalkyl.

54. The compound of any one of claims 1-53, or a pharmaceutically acceptable salt, solvate, tautomer, or stereoisomer thereof, wherein:
R⁷ is C₁-C₆alkyl.

55. The compound of any one of claims 1-54, or a pharmaceutically acceptable salt, solvate, tautomer, or stereoisomer thereof, wherein:
R⁸ is hydrogen or C₁-C₆alkyl.

56. The compound of any one of claims 1-55, or a pharmaceutically acceptable salt, solvate, tautomer, or stereoisomer thereof, wherein:
R⁸ is hydrogen.

57. The compound of any one of claims 1-56, or a pharmaceutically acceptable salt, solvate, tautomer, or stereoisomer thereof, wherein:
Ring B is aryl or heteroaryl.

58. The compound of any one of claims 1-57, or a pharmaceutically acceptable salt, solvate, tautomer, or stereoisomer thereof, wherein:
Ring B is phenyl.

59. The compound of any one of claims 1-58, or a pharmaceutically acceptable salt, solvate, tautomer, or stereoisomer thereof, wherein:
each R⁹ is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R^{9a}; or two R⁹ on the same atom are taken together to form an oxo.

60. The compound of any one of claims 1-59, or a pharmaceutically acceptable salt, solvate, tautomer, or stereoisomer thereof, wherein:
each R⁹ is independently deuterium, halogen, —CN, —C(═O)NR^{c}R^{d}, C₁-C₆alkyl, C₁-C₆haloalkyl, C₁-C₆deuteroalkyl, cycloalkyl, heterocycloalkyl, aryl, or heteroaryl; wherein the alkyl, cycloalkyl, heterocycloalkyl, aryl, and heteroaryl is optionally and independently substituted with one or more R^{9a}.

61. The compound of any one of claims 1-60, or a pharmaceutically acceptable salt, solvate, tautomer, or stereoisomer thereof, wherein:
each R⁹ is independently halogen or C₁-C₆alkyl.

62. The compound of any one of claims 1-61, or a pharmaceutically acceptable salt, solvate, tautomer, or stereoisomer thereof, wherein:
each R^{9a} is independently deuterium, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

63. The compound of any one of claims 1-62, or a pharmaceutically acceptable salt, solvate, tautomer, or stereoisomer thereof, wherein:
m is 1-3.

64. The compound of any one of claims 1-62, or a pharmaceutically acceptable salt, solvate, tautomer, or stereoisomer thereof, wherein:
m is 1 or 2.

65. A compound, or a pharmaceutically acceptable salt, solvate, tautomer, or stereoisomer thereof, selected from table 1.

66. A pharmaceutical composition comprising a compound of any one of claims 1-65, or a pharmaceutically acceptable salt, solvate, tautomer, or stereoisomer thereof, and a pharmaceutically acceptable excipient.

67. A method of treating cancer in a subject, comprising administering to the subject a compound of any one of claims 1-65, or a pharmaceutically acceptable salt, solvate, tautomer, or stereoisomer thereof, or a pharmaceutical composition of claim 66.

68. A method of inhibiting ribonucleotide reductase in a subject, comprising administering to the subject a compound of any one of claims 1-65, or a pharmaceutically acceptable salt, solvate, tautomer, or stereoisomer thereof, or a pharmaceutical composition of claim 66.

69. The method of claim 68, wherein the inhibition of ribonucleotide reductase occurs in a tumor cell in the subject in need thereof.

70. A method for treating a tumor or tumor cells in a subject, the method comprising administering a compound of any one of claims 1-65, or a pharmaceutically acceptable salt, solvate, tautomer, or stereoisomer thereof, in an amount sufficient to induce replication stress in the tumor or tumor cells; and administering a cancer-targeted therapeutic agent; wherein the tumor or tumor cells have an ecDNA signature; and wherein growth or size of the tumor or growth or number of tumor cells is reduced.

71. A method of treating an ecDNA-associated tumor or tumor cells comprising administering a compound of any one of claims 1-65, or a pharmaceutically acceptable salt, solvate, tautomer, or stereoisomer thereof, to a subject identified as having a tumor or tumor cells having ecDNA, wherein growth or size of the tumor or growth or number of the tumor cells is decreased as a result of treatment.

72. The method of claim 71, wherein the method further comprises administering a cancer-targeted therapeutic agent.

73. The method of claim 72, wherein the cancer-targeted therapeutic agent inhibits a gene or gene product comprised on ecDNA in the tumor or tumor cells.

74. A method for treating a tumor or tumor cells in a subject, the method comprising administering a compound of any one of claims 1-65, or a pharmaceutically acceptable salt, solvate, tautomer, or stereoisomer thereof, in an amount sufficient to induce replication stress in the tumor or tumor cells, wherein the tumor or tumor cells comprises ecDNA or have an ecDNA signature; and wherein growth or size of the tumor or growth or number of tumor cells is reduced.
